(19) United States
(12) Patent Application Publication
(10) Pub. No.: US 2015/0351390 A1
(43) Pub. Date: Dec. 10, 2015

(54) COMPOSITIONS AND METHODS FOR AUXIN-ANALOG CONJUGATION

(71) Applicant: PIONEER HI-BRED INTERNATIONAL, INC., Johnston, IA (US)

(72) Inventors: Linda A. Castle, Mountain View, CA (US); Li-Wei Chiu, San Jose, CA (US); Yumin Tao, Fremont, CA (US)

(21) Appl. No.: 14/654,218

(22) PCT Filed: Dec. 20, 2013
(86) PCT No.: PCT/US2013/076771

§ 371 Date: Jun. 19, 2015

Related U.S. Application Data
(60) Provisional application No. 61/740,759, filed on Dec. 21, 2012.
(60) Provisional application No. 61/777,045, filed on Mar. 12, 2013.
(60) Provisional application No. 61/781,996, filed on Mar. 14, 2013.

(51) Int. Cl. A01N 37/38; C12N 9/24; C12N 15/82; A01N 37/10

(52) U.S. Cl. CPC A01N 37/38; A01N 37/10; C12N 9/2402; C12N 15/8274; C12N 15/8294; C12Y 302/01

(57) ABSTRACT

Compositions and methods are provided to detoxify an auxin-analog herbicide through the use of at least one GH3 polypeptide having amino acid/auxin analog herbicide conjugation activity. Such GH3 polypeptides in the presence of an auxin-analog herbicide will produce an amino acid/auxin-analog conjugate having reduced herbicidal activity.

Various methods of employing the GH3 polypeptides and the polynucleotides encoding the same are provided. Such methods include methods to detoxify an auxin-analog herbicide comprising applying to a plant, a plant cell or a seed an auxin-analog herbicide, wherein the plant, plant cell or seed comprises a heterologous polynucleotide encoding a GH3 polypeptide, and expression of the GH3 polypeptide produces a non-herbicidal aspartate/auxin-analog conjugate or a glutamate/auxin-analog conjugate. Additional methods for controlling at least one weed in an area of cultivation comprising a crop or a seed of the crop are provided, as are methods of detoxifying a contaminated material having an auxin-analog herbicide.

### CROSS-REFERENCE TO RELATED APPLICATIONS

This application claims the benefit of U.S. Provisional Application No. 61/740,759, filed on Dec. 21, 2012; U.S. Provisional Application No. 61/777,045, filed on Mar. 12, 2013; and U.S. Provisional Application No. 61/781,996, filed on Mar. 14, 2013; each of which is incorporated herein by reference in its entirety.

### FIELD OF THE INVENTION

This invention is in the field of molecular biology. More specifically, this invention pertains to method and compositions to detoxify an auxin-analog herbicide through the use of a GH3 polypeptide or active variant or fragment thereof.

### REFERENCE TO A SEQUENCE LISTING SUBMITTED AS A TEXT FILE VIA EFS-WEB

The official copy of the sequence listing is submitted electronically via EFS-Web as an ASCII formatted sequence listing with a file named 431145SEQLIST.txt, created on Dec. 19, 2013, and having a size of 716 KB and is filed concurrently with the specification. The sequence listing contained in this ASCII formatted document is part of the specification and is herein incorporated by reference in its entirety.

### BACKGROUND OF THE INVENTION

In the commercial production of crops, it is desirable to easily and quickly eliminate unwanted plants (i.e., “weeds”) from a field of crop plants. An ideal treatment would be one which could be applied to an entire field but which would eliminate only the unwanted plants while leaving the crop plants unharmed. One such treatment system would involve the use of crop plants which are tolerant to a herbicide so that when the herbicide was sprayed on a field of herbicide-tolerant crop plants or an area of cultivation containing the crop, the crop plants would continue to thrive while non-herbicide-tolerant weeds were killed or severely damaged. Ideally, such treatment systems would take advantage of varying herbicide properties so that weed control could provide the best possible combination of flexibility and economy. For example, individual herbicides have different longevities in the field, and some herbicides persist and are effective for a relatively long time after they are applied to a field while other herbicides are quickly broken down into other and/or non-active compounds.

Crop tolerance to specific herbicides can be conferred by engineering genes into crops which encode appropriate herbicide metabolizing enzymes and/or insensitive herbicide targets. In some cases these enzymes, and the nucleic acids that encode them, originate in a plant. In other cases, they are derived from other organisms, such as microbes. See, e.g., Padgette et al. (1996) “New weed control opportunities: Development of soybeans with a Roundup Ready® gene” and Vasil (1996) “Phosphinothricin-resistant crops,” both in Herbicide-Resistant Crops, ed. Duke (CRC Press, Boca Raton, Fla.) pp. 54-84 and pp. 85-91. Indeed, transgenic plants have been engineered to express a variety of herbicide tolerance genes from a variety of organisms.

While a number of herbicide-tolerant crop plants are presently commercially available, improvements in every aspect of crop production, weed control options, extension of residual weed control, and improvement in crop yield are continuously in demand. Particularly, due to local and regional variation in dominant weed species, as well as, preferred crop species, a continuing need exists for customized systems of crop protection and weed management which can be adapted to the needs of a particular region, geography, and/or locality. A continuing need therefore exists for compositions and methods of crop protection and weed management.

### BRIEF SUMMARY OF THE INVENTION

Compositions and methods are provided to detoxify an auxin-analog herbicide through the use of at least one GH3 polypeptide having amino acid/auxin conjugation activity. Such GH3 polypeptides in the presence of an auxin-analog herbicide will produce an amino acid/auxin-analog conjugate having reduced herbicidal activity.

Various methods of employing the GH3 polypeptides and the polynucleotides encoding the same are provided. Such methods include methods to detoxify an auxin-analog herbicide comprising applying to a plant, a plant cell or a seed an auxin-analog herbicide, wherein the plant, plant cell or seed comprises a heterologous polynucleotide encoding a GH3 polypeptide having amino acid/auxin-analog conjugation activity, and wherein expression of the GH3 polypeptide produces an amino acid/auxin-analog conjugate having reduced herbicidal activity. Further methods include detoxifying a contaminated material having an auxin-analog herbicide by contacting the contaminated material with an effective amount of a host cell having the heterologous GH3 polypeptide.

Additional methods for controlling at least one weed in an area of cultivation comprising a crop or a seed of the crop are provided. The method comprises applying to the area of cultivation and/or applying to the crop or a seed of the crop in the area of cultivation a sufficient amount of an auxin-analog herbicide to control weeds without significantly affecting the crop, wherein the crop or seed thereof in the area of cultivation comprises a heterologous polynucleotide encoding a GH3 polypeptide having amino acid/auxin-analog conjugation activity.

### BRIEF DESCRIPTION OF THE DRAWINGS

FIG. 1 provides a schematic showing the formation of the aspartate/dicamba conjugate (A) and glutamate/dicamba conjugate (B) by a GH3 polypeptide having acyl-acid-amido synthetase activity.

FIG. 2 provides a schematic showing the formation of the aspartate/2,4-D conjugate (A) and glutamate/2,4-D conjugate (B) by a GH3 polypeptide having acyl-acid-amido synthetase activity.

FIG. 3 shows that soybean germination is not affected by auxin-analog conjugates of aspartate and glutamate.

FIGS. 4A, B and C provides the phylogenic relationship of the 246 GH3 polypeptides. The consensus tree was generated using CLUSTAL W and a bootstrap test with 1000 iterations (bootstrap values are indicated at each branch). Subgroups A, B, C were marked based on phylogenetic tree analysis.

FIG. 5 provides the phylogenic relationship of the 145 GH3 polypeptides using CLUSTAL W. The phylogenetic tree was inferred using the Neighbor-Joining method (Saitou and Nei (1987) Molecular Biology and Evolution 4:406-425). The optimal tree with the sum of branch length=17.20082290 is shown. The evolutionary distances were computed using the Poisson correction method (Zuckerkandl and Pauling (1965) In Evolving Genes and Proteins by Bryson and Vogel, pp. 97-166. Academic Press, New York) and are in the units of the number of amino acid substitutions per site. The analysis involved 145 amino acid sequences. All positions containing gaps and missing data were eliminated. There were a total of 112 positions in the final dataset. Evolutionary analyses were conducted in MEGA5 (Tamura et al. (2011) Molecular Biology and Evolution 28: 2731-2739). Subgroups A, B, and C were marked based on phylogenetic tree analysis. Circle: GH3 proteins tested for Asp and Glu conjugation activity with IAA, 2,4-D, and dicamba. Filled circle: GH3 proteins that are active 2,4-D conjugases with Asp and/or Glu.

FIG. 6 provides the phylogenic relationship of 78 activity tested GH3 polypeptides using CLUSTAL W. The phylogenetic tree was inferred using the Neighbor-Joining method (Saitou and Nei (1987) Molecular Biology and Evolution 4:406-425). The optimal tree with the sum of branch length=8.67906939 is shown. The tree is drawn to scale, with branch lengths in the same units as those of the evolutionary distances used to infer the phylogenetic tree. The evolutionary distances were computed using the Poisson correction method (Zuckerkandl and Pauling (1965) In Evolving Genes and Proteins by Bryson and Vogel, pp. 97-166. Academic Press, New York) and are in the units of the number of amino acid substitutions per site. All positions containing gaps and missing data were eliminated. There were a total of 212 positions in the final dataset. Evolutionary analyses were conducted in MEGA5 (Tamura et al., 2011. Molecular Biology and Evolution 28: 2731-2739). All 78 tested GH3 proteins have 2,4-D conjugation activity with Asp and/or Glu except ones marked with a filled diamond or a filled triangle. Open or filled diamond: GH3 proteins with no IAA conjugation activity to Asp or Glu; Open square: GH3 protein with dicamba conjugation activity to Glu. Subgroups A, B, and C were marked based on phylogenetic tree analysis.

FIG. 7 shows the dicamba-Glu conjugation activity of SEQ ID NO: 52 (PpGH3-2). PpGH3-2 is able to conjugate dicamba with glutamate. The amount of dicamba-Glu conjugates increases with increased amount of PpGH3-2 protein in the reaction.

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### I. Detoxified Auxin-Analog Herbicides

Methods and compositions are provided to detoxify auxin-analog herbicides via amino acid conjugation. As demonstrated herein, converting an auxin-analog herbicide into an amino acid/auxin conjugate, such as an aspartate/auxin-analog conjugate and/or a glutamate/auxin-analog conjugate, reduces the herbicidal activity of the auxin-analog herbicide. As discussed in further detail herein, amino acid conjugation to the auxin-analog herbicide can be achieved through the use of at least one GH3 polypeptide or an active variant or fragment thereof.

Methods and compositions are provided to detoxify an auxin-analog herbicide. As used herein, “detoxify” or “detoxifying” an auxin-analog herbicide comprises any modification to the auxin-analog herbicide which reduces the herbicidal effect of the compound. A “reduced” herbicidal effect comprises any statistically significant decrease in the sensitivity of the plant or plant cell to the modified auxin-analog. The reduced herbicidal activity of a modified auxin-analog herbicide can be assayed in a variety of ways including, for example, assaying for the decreased sensitivity of a plant, a plant cell, or plant explant to the presence of the modified auxin-analog. See, for example, Example 2 provided herein. In such instances, the plant, plant cell, or plant explant will display a decreased sensitivity to the modified auxin-analog when compared to a control plant, plant cell, or plant explant which was contacted with the non-modified auxin-analog herbicide. Thus, in one example, a “reduced herbicidal effect” is demonstrated when plants display the increased tolerance to a modified auxin-analog and a dose/response curve is shifted to the right when compared to when the non-modified auxin-analog herbicide is applied. Such dose/response curves have “dose” plotted on the x-axis and “percentage injury”, “herbicidal effect” etc. plotted on the y-axis.

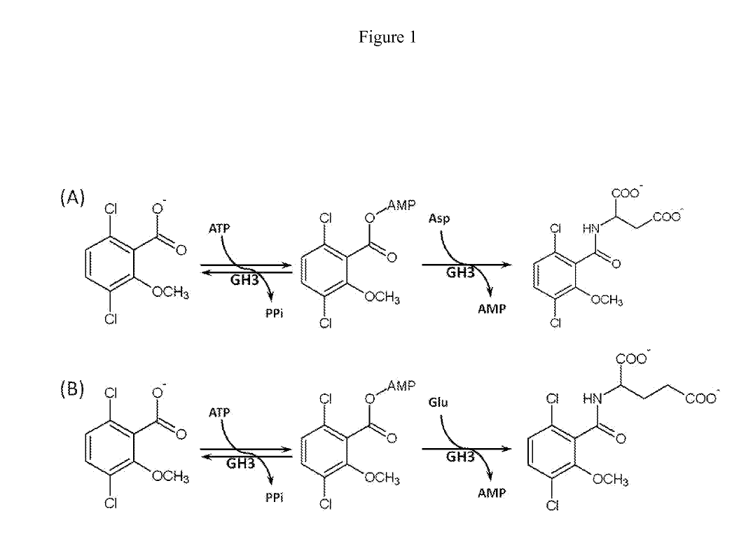

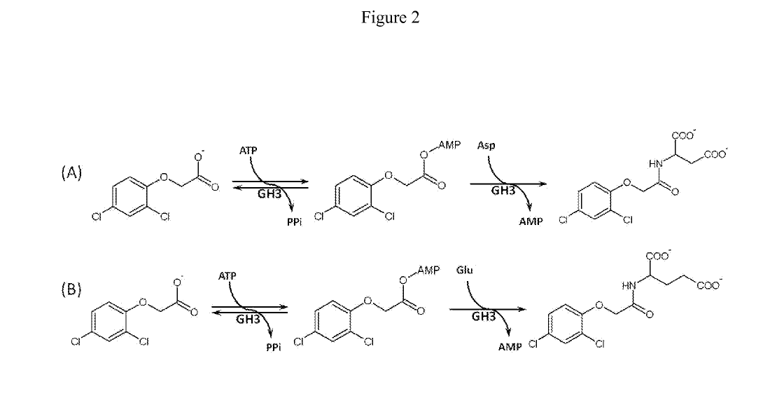

In one embodiment, methods and compositions are provided to detoxify an auxin-analog herbicide via amino acid conjugation. Auxin amino acid conjugation is a two-step enzymatic reaction. The first step involves adenylation: the transfer of AMP from ATP to the carboxylic acid group of an acyl substrate, forming an activated acyl-adenylate intermediate and releasing pyrophosphate (PPᵢ). The second step involves a transferase reaction replacing AMP of the intermediate with an amino acid by the formation of an amide bond. See FIGS. 1 and 2. In specific embodiments, the amino acid conjugated to the auxin-analog herbicide comprises an aspartate and/or a glutamate amino acid. In such embodiments, the detoxified auxin-analog comprises an aspartate/auxin-analog conjugate and/or a glutamate/auxin-analog conjugate. In still further embodiments, the detoxified auxin-analog comprises an aspartate/2,4-D conjugate and/or a glutamate/2,4-D conjugate, while in other embodiments, the detoxified auxin-analog comprises an aspartate/dicamba conjugate and/or a glutamate/dicamba conjugate. As used herein, the term “aspartate” and “glutamate” refer to the acid or the ester form of the amino acid. One of skill will recognize that the formation of the ester or the acid will depend on the conditions in which the amino acid is found.

It is further recognized that the amino acid conjugated to the auxin-analog herbicide need not be an aspartate and/or a glutamate amino acid. In some embodiments, the amino acid conjugated to the auxin-analog (including a 2,4-D conjugate or a dicamba conjugate) can comprises a histidine, isoleucine, leucine, lysine, methionine, phenylalanine, threonine, valine, tryptophan, alanine, tyrosine, glycine, glutamine, cysteine, asparagine, arginine, serine, and/or proline amino acid, or any combination thereof.

As used herein, an “auxin-analog herbicide” or “synthetic auxin herbicide” are used interchangeably and comprises any auxinic or growth regulator herbicides, otherwise known as Group 4 herbicides (based on their mode of action). These types of herbicides mimic or act like the natural plant growth regulators called auxins. The action of auxin-analog herbicide appears to affect cell wall plasticity and nucleic acid metabolism, which can lead to uncontrolled cell division and growth. See, for example, Cox et al. (1994) Journal of Pesticide Reform 14:30-35; Dayan et al. (2010) Weed Science 58:340-350; Davidonis et al. (1982) Plant Physiol 70:357-360; Mithila et al. (2011) Weed Science 59:445-457; Grossmann (2007) Plant Signalling and Behavior 2:421-423, U.S. Pat. No. 7,855,326; US App. Pub. 2012/0178627; US App. Pub. 2011/0124503; and U.S. Pat. No. 7,838,733, each of which is herein incorporated by reference.

Auxin-analog herbicides include the chemical families: phenoxy, carboxylic acid (or pyridine), benzoic acid, quinaline carboxylic acid, and aminocyclopyrachlor (MAT28). Phenoxy herbicides are most common and have been used as herbicides. One example of a phenoxy herbicide includes (2,4-dichlorophenoxy)acetic acid, otherwise known as 2,4-D. Other examples include 4-(2,4-dichlorophenoxy)butyric acid (2,4-DB), 2-(2,4-dichlorophenoxy)propanoic acid (2,4-DP), (2,4,5-trichlorophenoxy)acetic acid (2,4,5-T), 2-(2,4,5-Trichlorophenoxyl)Propionic Acid (2,4,5-TP), 2-(2,4-dichloro-3-methylphenoxy)-N-phenylpropanamide (clomeprop), (4-chloro-2-methylphenoxy)acetic acid (MCPA), 4-(4-chloro-o-tolyloxy)butyric acid (MCPB), and 2-(4-chloro-2-methylphenoxy)propanoic acid (MCPP).

The next largest chemical family is the carboxylic acid herbicides, also called pyridine herbicides. Examples include 3,6-dichloro-2-pyridinecarboxylic acid (Clopyralid), 4-amino-3,5,6-trichloro-2-pyridinecarboxylic acid (picloram), (2,4,5-trichlorophenoxy) acetic acid (triclopyr), and 4-amino-3,5-dichloro-6-fluoro-2-pyridyloxyacetic acid (fluoroxypyr).

Examples of benzoic acids include 3,6-dichloro-o-anisic acid (dicamba) and 3-amino-2,5-dichlorobenzoic acid (choramben). Dicamba is a particularly useful herbicide for use in the methods and compositions disclosed herein.

A fourth chemical family of auxin-analog herbicide is the quinaline carboxylic acid family. Example includes 3,7-dichloro-8-quinolinecarboxylic acid (quinclorac). This herbicide is unique in that it also will control some grass weeds, unlike the other auxin-analog herbicide which essentially control only broadleaf or dicotyledonous plants. The other herbicide in this category is 7-chloro-3-methyl-8-quinolinecarboxylic acid (quinmerac). In other embodiments, the auxin-analog herbicide comprises aminocyclopyrachlor, aminopyralid benazolin-ethyl, chloramben, clomeprop, clopyralid, dicamba, 2,4-D, 2,4-DB, dichlorprop, fluroxypyr, mecoprop, MCPA, MCPB, 2,3,6-TBA, picloram, triclopyr, quinclorac, or quinmerac. See, for example, WO2010/046422, WO2011/161131, WO2012/033548, and US Application Publications 20110287935, 20100069248, and 20100048399, each of which is herein incorporated by reference in their entirety.

While any auxin-analog herbicide can be employed in the methods and compositions disclosed herein, in one embodiment, the auxin-analog herbicide comprises dicamba or 2,4-D, and the detoxified auxin-analog herbicide comprises an aspartate/dicamba conjugate, a glutamate/dicamba conjugate, an aspartate/2,4-D conjugate and/or a glutamate/2,4-D conjugate.

### II. Methods of Forming an Amino Acid/Auxin-Analog Conjugate

For auxin-analog herbicides, the synthesis and degradation of amide conjugates was not known, particularly because 2,4-D and dicamba were not thought to be substrates of acyl amide synthetases (Staswick et al. (2005) Plant Cell 17:616-627; Chen et al. (2010) J Biol Chem 285:29780-29786) and such conjugates have not been found from plants. As demonstrated herein, auxin-analog herbicides can form amide conjugates and the conjugated aspartate/auxin-analogs and/or a glutamate/auxin-analogs are shown herein to display reduced herbicidal activity in plants.

i. GH3 Polypeptides and Polynucleotides Encoding the Same

Various methods can be employed to form the auxin-analog herbicide/amino acid conjugate. In one embodiment, a GH3 polypeptide is employed. The GH3 protein family comprises polypeptides having acyl-acid-amido synthetase activity and which catalyze the ATP-dependent formation of amino acid conjugates to modulate levels of at least one active plant compound, including, for example, auxin and/or jasmonates and/or benzoate substrates. The GH3 polypeptide employed in the methods and compositions disclosed herein will have acyl-acid-amido synthetase activity and catalyze, at least the formation of an amino acid/auxin-analog conjugate having reduced herbicidal activity. In more specific embodiments, the GH3 polypeptide having acyl-acid-amido activity will catalyze the formation of a glutamate/auxin-analog conjugate, an aspartate/auxin-analog conjugate, a glutamate/dicamba conjugate, an aspartate/dicamba conjugate, a glutamate/2,4-D conjugate and/or an aspartate/2,4-D conjugate; wherein the auxin-analog conjugate has reduced herbicidal activity.

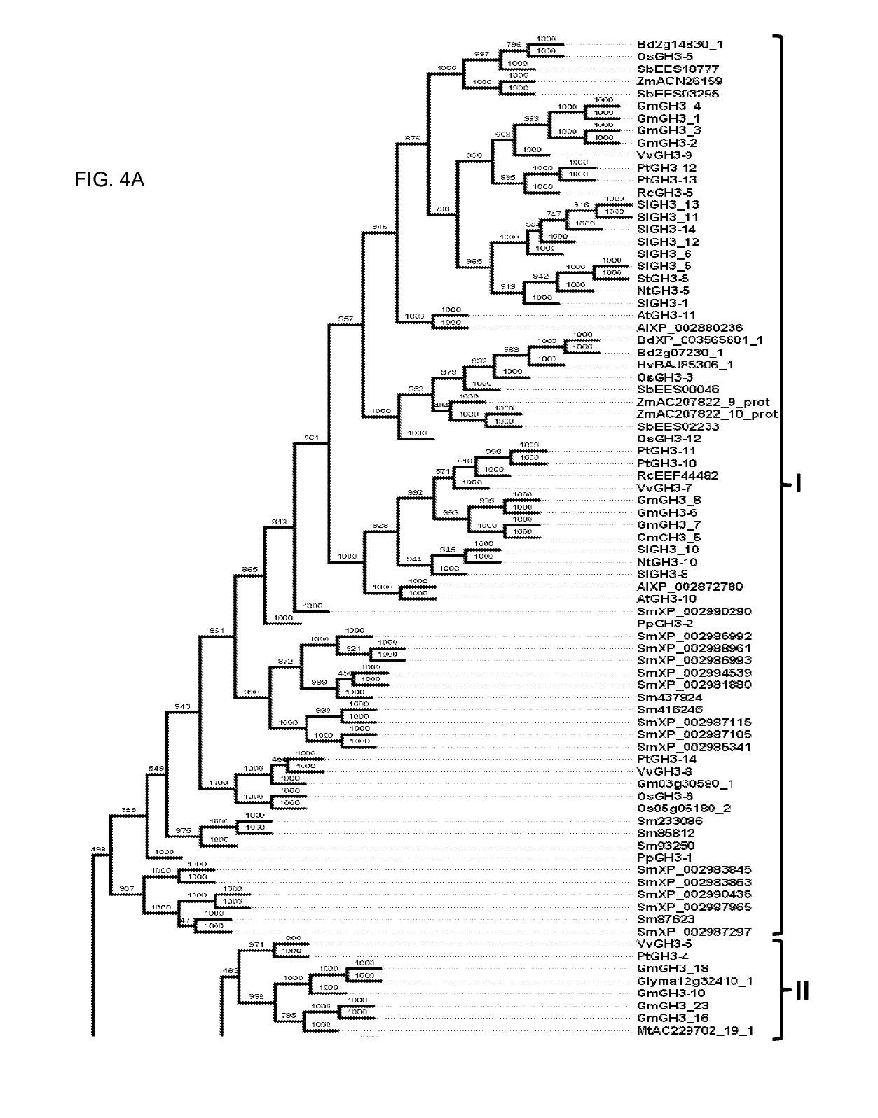

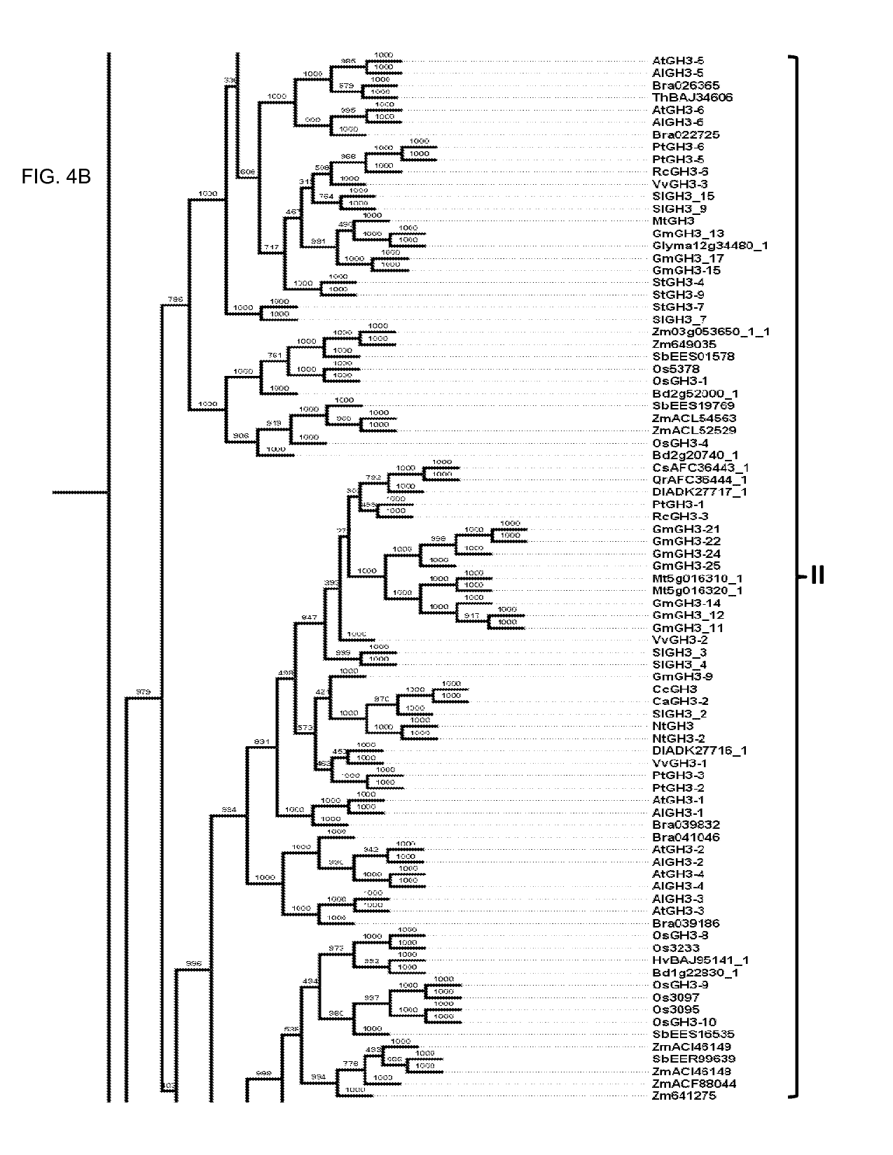

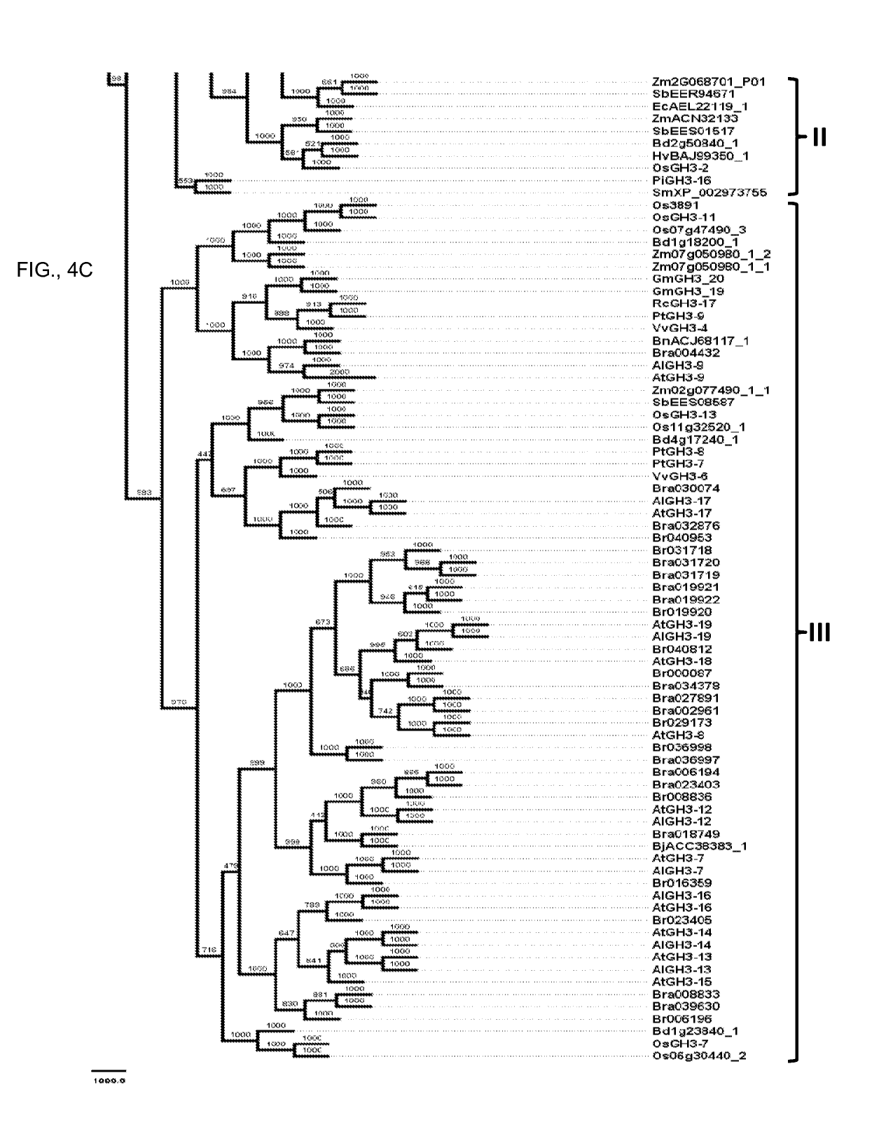

Various GH3 polypeptides are known, previous studies of the substrate specificity of GH3 family of proteins has resulted in the GH3 protein family being characterized into three major subgroups. See, Staswick et al. (2002) Plant Cell 14: 1405-1415 and Wang et al. (2008) Plant Growth Regul 56:225-232, both of which are herein incorporated by reference. Subgroup I of the GH3 polypeptides catalyzes the ligation of amino acids to jasmonic acid (Staswick et al. (2002) Plant Cell 14: 1405-1415, herein incorporated by reference). Auxins such as IAA, PAA, IBA and salicylic acid are the substrates of subgroup II (Staswick et al. (2002) Plant Cell 14: 1405-1415; Staswick et al. (2005) Plant Cell 17:616-627, both of which are incorporated by reference). Subgroup III protein AtGH3-12 can conjugate 4-hydroxybenzoate, and other benzoates (Okrent et al. (2009) J Biol Chem 284:9742-9754). In another embodiment, the phylogenetic diversity of various GH3 polypeptides (Groups A, B and C) is set forth in FIGS. 4, 5 and 6.

Non-limiting examples of various GH3 enzymes are provided herein. Group I GH3 polypeptides include, for example, SEQ ID NOS: 15, 16, 17, 51, 52, 53, 54, 55, 56, 57, 59, 60, 61, 62, 63, 64, 65, 66, 67, 98, 99, 100, 101, 102, 103, 104, 105, 106, 107, 108, 109, 110, 111, 112, 113, 114, 115, 116, 122, 134, 135, 136, 137, 138, 139, 140, 141, and active variants and fragment thereof. Group II GH3 polypeptides include, for example, SEQ ID NOS: 1, 2, 3, 4, 5, 6, 7, 8, 9, 10, 11, 12, 13, 14, 33, 34, 35, 36, 37, 38, 39, 40, 41, 42, 43, 44, 45, 46, 47, 48, 49, 50, 58, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 117, 118, 119, 120, 121, 124, 142, 144, 145, and active variants and fragments thereof. Group III GH3 polypeptides include, for example, SEQ ID NOS: 18, 19, 20, 21, 22, 23, 24, 25, 26, 27, 28, 29, 30, 31, 32, 123, 125, 126, 127, 128, 129, 130, 131, 132, 133, 143, and active variants and fragments thereof. Further provided are the polynucleotides encoding these various polypeptides and active variants and fragments thereof.

ii. Active Fragments and Variants of GH3 Sequences

Methods and compositions are provided which employ GH3 polypeptide having acyl-acid-amido activity that catalyze the formation of an amino acid/auxin-analog conjugate, such as a glutamate/auxin-analog conjugate and/or an aspartate/auxin-analog conjugate, wherein the auxin-analog conjugate has reduced herbicidal activity.

iii. Polynucleotide and Polypeptide Fragments

Fragments and variants of GH3 polynucleotides and polypeptides can be employed in the methods and compositions disclosed herein. By “fragment” is intended a portion of the polynucleotide or a portion of the amino acid sequence and hence protein encoded thereby. Fragments of a polynucleotide may encode protein fragments that retain the acyl-acid-amido activity and the ability to catalyze the formation of a glutamate/auxin-analog conjugate, and/or an aspartate/auxin-analog conjugate, wherein the auxin-analog conjugate has reduced herbicidal activity. Thus, fragments of a nucleotide sequence may range from at least about 20 nucleotides, about 50 nucleotides, about 100 nucleotides, and up to the full-length polynucleotide encoding the GH3 polypeptides.

A fragment of a GH3 polynucleotide that encodes a biologically active portion of a GH3 polypeptide will encode at least 50, 75, 100, 150, 175, 200, 225, 250, 275, 300, 325, 350, 375, 400, 410, 415, 420, 425, 430, 435, 440, 480, 500, 550, 600, 620 contiguous amino acids, or up to the total number of amino acids present in a full-length GH3 polypeptide as set forth in, for example, SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, or 145.

Thus, a fragment of a GH3 polynucleotide encodes a biologically active portion of a GH3 polypeptide. A biologically active portion of a GH3 polypeptide can be prepared by isolating a portion of one of the polynucleotides encoding a GH3 polypeptide, expressing the encoded portion of the GH3 polypeptides (e.g., by recombinant expression in vitro), and assessing for acyl-acid-amido activity and the ability to catalyze the formation of a glutamate/auxin-analog conjugate, and/or an aspartate/auxin-analog conjugate, wherein the amino acid auxin-analog conjugate has reduced herbicidal activity. Polynucleotides that are fragments of a GH3 nucleotide sequence comprise at least 16, 20, 50, 75, 100, 150, 200, 250, 300, 350, 400, 450, 500, 550, 600, 650, 700, 800, 900, 1,000, 1,100, 1,200, 1,300, or 1,400 contiguous nucleotides, or up to the number of nucleotides present in a full-length Polynucleotide encoding a GH3 polypeptide disclosed herein.

iv. Polynucleotide and Polypeptide Variants

“Variant” protein is intended to mean a protein derived from the protein by deletion (i.e., truncation at the 5′ and/or 3′ end) and/or a deletion or addition of one or more amino acids at one or more internal sites in the native protein and/or substitution of one or more amino acids at one or more sites in the native protein. Variant proteins encompassed are biologically active, that is they continue to possess the desired biological activity of the native protein, that is, having acyl-acid-amido activity and the ability to catalyze the formation of an amino acid/auxin conjugate, such as a glutamate/auxin-analog conjugate and/or an aspartate/auxin-analog conjugate, wherein the amino acid/auxin-analog conjugate has reduced herbicidal activity.

“Variants” is intended to mean substantially similar sequences. For polynucleotides, a variant comprises a polynucleotide having a deletion (i.e., truncations) at the 5′ and/or 3′ end and/or a deletion and/or addition of one or more nucleotides at one or more internal sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the GH3 polypeptides. Naturally occurring variants such as these can be identified with the use of well-known molecular biology techniques, as, for example, with polymerase chain reaction (PCR) and hybridization techniques, and sequencing techniques as outlined below. Variant polynucleotides also include synthetically derived polynucleotides, such as those generated, for example, by using site-directed mutagenesis or gene synthesis but which still encode a GH3 polypeptide.

Biologically active variants of a GH3 polypeptide (and the polynucleotide encoding the same) will have at least about 80%, 85%, 86%, 87%, 88%, 89%, 90%, 91%, 92%, 93%, 94%, 95%, 95.7%, 96%, 97%, 98%, 99%, or more sequence identity to the polypeptide of any one of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, or 145 as determined by sequence alignment programs and parameters described elsewhere herein.

The GH3 polypeptides and the active variants and fragments thereof may be altered in various ways including amino acid substitutions, deletions, truncations, and insertions. Methods for such manipulations are generally known in the art. For example, amino acid sequence variants and fragments of the GH3 polypeptides can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found. Washington, D.C.), herein incorporated by reference. Conservative substitutions, such as exchanging one amino acid with another having similar properties, may be optimal.

Obviously, the mutations that will be made in the DNA encoding the variant must not place the sequence out of reading frame and optimally will not create complementary regions that could produce secondary mRNA structure. See, EP Patent Application Publication No. 75,444.

### III. Host Cells, Plants and Plant Parts

Host cells, plants, plant cells, plant parts and seeds, and grain having a heterologous copy of the GH3 sequences disclosed herein are provided. It is expected that those of skill in the art are knowledgeable in the numerous systems available for the introduction of a polypeptide or a nucleotide sequence disclosed herein into a host cell. No attempt to describe in detail the various methods known for providing sequences in prokaryotes or eukaryotes will be made.

By “host cell” is meant a cell which comprises a heterologous GH3 sequence. Host cells may be prokaryotic cells, such as E. coli, or eukaryotic cells such as yeast cells. Host cells can also be monocotyledonous or dicotyledonous plant cells.

In specific embodiments, the host cells, plants and/or plant parts have stably incorporated at least one heterologous polynucleotide encoding a GH3 polypeptide or an active variant or fragment thereof. Thus, host cells, plants, plant cells, plant parts and seed are provided which comprise at least one heterologous polynucleotide encoding a GH3 polypeptide of any one of SEQ ID NOS: SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 74, 65, 66, 67, 68, 69, 70, 71, 72, 73, 74, 75, 76, 77, 78, 79, 80, 81, 82, 83, 84, 85, 86, 87, 88, 89, 90, 61, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, or 145 and/or an active fragment and/or variant thereof. The GH3 polypeptides have acyl-acid-amido activity, and therefore have the ability to catalyze the formation of an amino acid/auxin-analog conjugate, such as a glutamate/auxin-analog conjugate and/or an aspartate/auxin-analog conjugate, wherein the amino acid/auxin-analog conjugate has reduced herbicidal activity.

Thus, the host cell, plants, plant cells and seed which express the heterologous polynucleotide encoding the GH3 polypeptide can display an increased tolerance to the auxin-analog herbicide. “Increased tolerance” to a herbicide is demonstrated when plants which display the increased tolerance to a herbicide are subjected to the auxin-analog herbicide and a dose/response curve is shifted to the right when compared with that provided by an appropriate control plant. Such dose/response curves have “dose” plotted on the x-axis and “percentage injury”, “herbicidal effect” etc. plotted on the y-axis. Plants which are substantially “resistant” or “tolerant” to the herbicide exhibit few, if any, significant negative agronomic effects when subjected to the herbicide at concentrations and rates which are typically employed by the agricultural community to kill weeds in the field.

In specific embodiments, the heterologous polynucleotide encoding the GH3 polypeptide or active variant or fragment thereof in the host cell, plant or plant part is operably linked to a constitutive, tissue-preferred, or other promoter for expression in the host cell or the plant of interest.

As used herein, the term plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

The polynucleotide encoding the GH3 polypeptide and active variants and fragments thereof may be used for transformation of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis), and Poplar and Eucalyptus. In specific embodiments, plants of the present invention are crop plants (for example, corn, alfalfa, sunflower, Brassica, soybean, cotton, safflower, peanut, sorghum, wheat, millet, tobacco, etc.).

In other embodiments, corn and soybean plants are of interest.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mung bean, lima bean, fava bean, lentils, chickpea, etc.

A “subject plant or plant cell” is one in which genetic alteration, such as transformation, has been affected as to a gene of interest, or is a plant or plant cell which is descended from a plant or cell so altered and which comprises the alteration. A “control” or “control plant” or “control plant cell” provides a reference point for measuring changes in phenotype of the subject plant or plant cell.

A control plant or plant cell may comprise, for example: (a) a wild-type plant or cell, i.e., of the same germplasm, variety or line as the starting material for the genetic alteration which resulted in the subject plant or cell; (b) a plant or plant cell of the same genotype as the starting material but which has been transformed with a null construct (i.e. with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest; or (e) the subject plant or plant cell itself, under conditions in which the gene of interest is not expressed.

### IV. Polynucleotide Constructs

The use of the term “polynucleotide” is not intended to limit the methods and compositions to polynucleotides comprising DNA. Those of ordinary skill in the art will recognize that polynucleotides can comprise ribonucleotides and combinations of ribonucleotides and deoxyribonucleotides. Such deoxyribonucleotides and ribonucleotides include both naturally occurring molecules and synthetic analogues. The polynucleotides employed herein also encompass all forms of sequences including, but not limited to, single-stranded forms, double-stranded forms, hairpins, stem-and-loop structures, and the like.

The polynucleotides encoding a GH3 polypeptide or active variant or fragment thereof can be provided in expression cassettes for expression in the plant of interest. The cassette can include 5′ and 3′ regulatory sequences operably linked to a polynucleotide encoding a GH3 polypeptide or an active variant or fragment thereof “Operably linked” is intended to mean a functional linkage between two or more elements. For example, an operable linkage between a polynucleotide of interest and a regulatory sequence (i.e., a promoter) is a functional link that allows for expression of the polynucleotide of interest. Operably linked elements may be contiguous or non-contiguous. When used to refer to the joining of two protein coding regions, by operably linked is intended that the coding regions are in the same reading frame. Additional gene(s) can be provided on multiple expression cassettes. Such an expression cassette is provided with a plurality of restriction sites and/or recombination sites for insertion of the polynucleotide encoding a GH3 polypeptide or an active variant or fragment thereof to be under the transcriptional regulation of the regulatory regions.

The expression cassette can include in the 5′-3′ direction of transcription, a transcriptional and translational initiation region (i.e., a promoter), a polynucleotide encoding a GH3 polypeptide or an active variant or fragment thereof, and a transcriptional and translational termination region (i.e., termination region) functional in plants. The regulatory regions (i.e., promoters, transcriptional regulatory regions, and translational termination regions) and/or the polynucleotide encoding a GH3 polypeptide or an active variant or fragment thereof may be native/analogous to the host cell or to each other. Alternatively, the regulatory regions and/or the polynucleotide encoding the GH3 polypeptide of or an active variant or fragment thereof may be heterologous to the host cell or to each other. Moreover, as discussed in further detail elsewhere herein, the polynucleotide encoding the GH3 polypeptide can further comprise a polynucleotide encoding a “targeting signal” that will direct the GH3 polypeptide to a desired sub-cellular location.

As used herein, “heterologous” in reference to a sequence is a sequence that originates from a foreign species, or, if from the same species, is modified from its native form in composition and/or genomic locus by deliberate human intervention. For example, a promoter operably linked to a heterologous polynucleotide is from a species different from the species from which the polynucleotide was derived, or, if from the same/analogous species, one or both are modified from their original form and/or genomic locus, or the promoter is not the native promoter for the operably linked polynucleotide.

While it may be optimal to express the sequences using heterologous promoters, the native promoter sequences may be used. Such constructs can change expression levels of the polynucleotide encoding a GH3 polypeptide in the host cell, plant or plant cell. Thus, the phenotype of the host cell, plant or plant cell can be altered.

The termination region may be native with the transcriptional initiation region, may be native with the operably linked polynucleotide encoding a GH3 polypeptide or active variant or fragment thereof, may be native with the host cell (i.e., plant cell), or may be derived from another source (i.e., foreign or heterologous) to the promoter, the Polynucleotide encoding a GH3 polypeptide or active fragment or variant thereof, the plant host, or any combination thereof. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Where appropriate, the polynucleotides may be optimized for increased expression in the transformed host cell (i.e., a plant cell). In specific embodiments, the polynucleotides can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385. See also, Della-Cioppa et al. (1987) Plant Physiol. 84:965-968.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used to express the various GH3 sequences disclosed herein, including the native promoter of the polynucleotide sequence of interest. The promoters can be selected based on the desired outcome. Such promoters include, for example, constitutive, tissue-preferred, or other promoters for expression in plants.

Constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026); and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Tissue-preferred promoters can be utilized to target enhanced expression of the polynucleotide encoding the GH3 polypeptide within a particular plant tissue. Tissue-preferred promoters include those described in Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

Leaf-preferred promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590.

Meristem-preferred promoters can also be employed. Such promoter can drive expression in meristematic tissue, including, for example, the apical meristem, axillary buds, root meristems, cotyledon meristem and/or hypocotyl meristem. Non-limiting examples of meristem-preferred promoters include the shoot meristem specific promoter such as the Arabidopsis UFO gene promoter (Unusual Floral Organ) (USA6239329), the meristem-specific promoters of FTM1, 2, 3 and SVP1, 2, 3 genes as discussed in US Patent App. 20120255064, and the shoot meristem-specific promoter disclosed in U.S. Pat. No. 5,880,330. Each of these references is herein incorporated by reference in their entirety.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glyphosate, glufosinate ammonium, bromoxynil, sulfonylureas. Additional selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004) J. Cell Science 117:943-54 and Kato et al. (2002) Plant Physiol 129:913-42), and yellow florescent protein (PhiYFP™ from Evrogen, see, Bolte et al. (2004) J. Cell Science 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Sci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference. The above list of selectable marker genes is not meant to be limiting.

While the expression of a GH3 polypeptide or active variant or fragment thereof may be targeted to specific plant tissues or cell types through the use of appropriate promoters, it may also be targeted to different locations within the cell through the use of targeting information or “targeting labels.” Unlike the promoter, which acts at the transcriptional level, such targeting information is part of the initial translation product. Thus, in specific embodiments, the various GH3 polypeptides or active variants and fragments thereof, are expressed such that the GH3 polypeptide is targeted to a sub-cellular location, such as a plastid, a chloroplast, a vacuole, the endoplasmic reticulum (ER), a mitochondria, and/or the nucleus.

For example, one may produce a protein preceded by a signal peptide, which directs the translation product into the endoplasmic reticulum, by including in the construct (i.e. expression cassette) sequences encoding a signal peptide (such sequences may also be called the “signal sequence”). The signal sequence could be, for example, one associated with the gene encoding the GH3 polypeptide, or it may be taken from another gene and thereby be heterologous to the GH3 sequence. There are many signal peptides described in the literature. See, for example, Raikhel and Chrispeels, “Protein sorting and vesicle traffic” in Buchanan et al., eds., (2000) Biochemistry and Molecular Biology of Plants (American Society of Plant Physiologists, Rockville, Md.), herein incorporated by reference. The addition of a signal peptide will result in the translation product entering the endoplasmic reticulum (in the process of which the signal peptide itself is removed from the polypeptide). The final intracellular location of the protein depends on other factors, which may be manipulated to result in a desired cellular for the GH3 polypeptide or active variant or fragment thereof. The default pathway, that is, the pathway taken by the polypeptide if no other targeting labels are included, results in secretion of the polypeptide across the cell membrane into the apoplast. The apoplast is the region outside the plasma membrane system and includes cell walls, intercellular spaces, and the xylem vessels that form a continuous, permeable system through which water and solutes may move.

In specific embodiments, the GH3 polypeptide or active variant or fragment thereof is located within the cell rather than outside the cell membrane. This can be accomplished, for example, by adding a polynucleotide encoding an endoplasmic reticulum retention signal sequence to the sequence of the GH3 polypeptide. Methods and sequences for doing this are described in Raikhel and Chrispeels, supra; for example, adding sequences encoding the amino acids K, D, E and L in that order, or variations thereof described in the literature, to the end of the protein coding portion of the polypeptide will accomplish this. ER retention sequences are well known in the art and include those set forth in U.S. Pat. No. 7,772,370 and, for example, in Denecke et al. (1992). EMBO J. 11:2345-2355; Wandelt et al. (1992) Plant J. 2:181-192; Denecke et al. (1993) J. Exp. Bot. 44:213-221; Vitale et al. (1993) J. Exp. Bot. 44:1417-1444; Gomord et al. (1996) Plant Physiol. Biochem. 34:165-181; Lehmann et al. (2001) Plant Physiol. 127 (2): 436-449.

Alternatively, the use of vacuolar targeting labels such as those described by Raikhel and Chrispeels, supra, in addition to a signal peptide will result in localization of the peptide in a vacuolar structure. As described in Raikhel and Chrispeels, supra, the vacuolar targeting label may be placed in different positions in the construct.

Use of a plastid transit peptide encoding sequence instead of a signal peptide encoding sequence will result in localization of the polypeptide in the plastid of the cell type chosen (Raikhel and Chrispeels, supra). Such transit peptides are known in the art. See, for example, Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Comm. 196:1414-1421; and Shah et al. (1986) Science 233:478-481. Chloroplast targeting sequences that encode such transit peptides are also known in the art and include the chloroplast small subunit of ribulose-1,5-bisphosphate carboxylase (Rubisco) (de Castro Silva Filho et al. (1996) Plant Mol. Biol. 30:769-780; Schnell et al. (1991) J. Biol. Chem. 266(5):3335-3342); 5-(enolpyruvyl)shikimate-3-phosphate synthase (EPSPS) (Archer et al. (1990) J. Bioenerg. Biomemb. 22(6):789-810); tryptophan synthase (Zhao et al. (1995) J. Biol. Chem. 270(11):6081-6087); plastocyanin (Lawrence et al. (1997) J. Biol. Chem. 272(33):20357-20363); chorismate synthase (Schmidt et al. (1993) J. Biol. Chem. 268(36):27447-27457); and the light harvesting chlorophyll a/b binding protein (LHBP) (Lamppa et al. (1988) J. Biol. Chem. 263:14996-14999), and the various chloroplast targeting sequences set forth in US App. Publication 2012-0304336, each of which is herein incorporated by reference.

One could also envision localizing the polypeptide in other cellular compartments by addition of suitable targeting information. (Raikhel and Chrispeels, supra). A useful site that provides information and references regarding recognition of the various targeting sequences can be found at: psort.nibb.acjp/mit. Other references regarding the state of the art of protein targeting include Silva-Filho (2003) Curr. Opin. Plant Biol. 6:589-595; Nicchitta (2002) Curr. Opin. Cell Biol. 14:412-416; Bruce (2001) Biochim Biophys Acta 1541: 2-21; Hadlington & Denecke (2000) Curr. Opin. Plant Biol. 3: 461-468; Emanuelsson et al. (2000) J Mol. Biol. 300: 1005-1016; Emanuelsson & von Heijne (2001) Biochim Biophys Acta 1541: 114-119, each of which is herein incorporated by reference.

### V. Stacking Other Traits of Interest

In some embodiments, the polynucleotide encoding the GH3 polypeptide or an active variant or fragment thereof are engineered into a molecular stack. Thus, the various host cells, plants, plant cells and seeds disclosed herein can further comprise one or more traits of interest, and in more specific embodiments, the host cell, plant, plant part or plant cell is stacked with any combination of polynucleotide sequences of interest in order to create plants with a desired combination of traits. As used herein, the term “stacked” includes having the multiple traits present in the same plant (i.e., both traits are incorporated into the nuclear genome, one trait is incorporated into the nuclear genome and one trait is incorporated into the genome of a plastid, or both traits are incorporated into the genome of a plastid). In one non-limiting example, “stacked traits” comprise a molecular stack where the sequences are physically adjacent to each other. A trait, as used herein, refers to the phenotype derived from a particular sequence or groups of sequences. In one embodiment, the molecular stack comprises at least one additional polynucleotide that confers tolerance to at least one additional auxin-analog herbicide and/or at least one additional polynucleotide that confers tolerance to a second herbicide.

Thus, in one embodiment, the host cell, plants, plant cells or plant part having the polynucleotide encoding the GH3 polypeptide or an active variant or fragment thereof is stacked with at least one other GH3 sequence. Alternatively, the host cell, plant, plant cells or seed having the heterologous polynucleotide encoding the GH3 polypeptide can have the GH3 sequence stacked with an additional sequence that confers tolerance to an auxin-analog herbicide via a different mode of action than that of the GH3 sequence. Such sequences include, but are not limited to, the aryloxyalkanoate dioxygenase polynucleotides which confer tolerance to 2,4-D and other phenoxy auxin herbicides, as well as, to aryloxyphenoxypropionate herbicides as described, for example, in WO2005/107437. Additional sequence can further include dicamba-tolerance polynucleotides as described, for example, in Herman et al. (2005) J. Biol. Chem. 280: 24759-24767, U.S. Pat. Nos. 7,820,883; 8,088,979; 8,071,874; 8,119,380; 7,105,724; 7,855,3326; 8,084,666; 7,838,729; 5,670,454; US Application Publications 2012/0064539, 2012/0064540, 2011/0016591, 2007/0220629, 2001/0016890, 2003/0115626, WO2012/094555, WO2007/46706, WO2012/024853, EP0716808, and EP1379539, and an acetyl coenzyme A carboxylase (ACCase) polypeptides, each of which is herein incorporated by reference.

In still other embodiments, host cells, plants, plant cells, explants and expression cassettes comprising the polynucleotide encoding the GH3 polypeptide or active variant or fragment thereof are stacked with a sequence that confers tolerance to HPPD inhibitors. For example, a P450 sequence could be employed which provides tolerance to HPPD-inhibitors by metabolism of the herbicide. Such sequences include, but are not limited to, the NSF1 gene. See, US 2007/0214515 and US 2008/0052797, both of which are herein incorporated by reference in their entirety. Additional HPPD target site genes that confer herbicide tolerance to plants include those set forth in U.S. Pat. Nos. 6,245,968 B1; 6,268,549; and 6,069,115; international publication WO 99/23886, US App Pub. 2012-0042413 and US App Pub 2012-0042414, each of which is herein incorporated by reference.

In some embodiments, the host cell, plant or plant cell having the heterologous polynucleotide encoding a GH3 polypeptide or active variant or fragment thereof may be stacked with sequences that confer tolerance to glyphosate such as, for example, glyphosate N-acetyltransferase. See, for example, WO02/36782, US Publication 2004/0082770 and WO 2005/012515, U.S. Pat. No. 7,462,481, U.S. Pat. No. 7,405,074, each of which is herein incorporated by reference. Additional glyphosate-tolerance traits include a sequence that encodes a glyphosate oxido-reductase enzyme as described more fully in U.S. Pat. Nos. 5,776,760 and 5,463,175. Other traits that could be combined with the polynucleotide encoding the GH3 polypeptide or active variant or fragment thereof include those derived from polynucleotides that confer on the plant the capacity to produce a higher level or glyphosate insensitive 5-enolpyruvylshikimate-3-phosphate synthase (EPSPS), for example, as more fully described in U.S. Pat. Nos. 6,248,876 B1; 5,627,061; 5,804,425; 5,633,435; 5,145,783; 4,971,908; 5,312,910; 5,188,642; 4,940,835; 5,866,775; 6,225,114 B1; 6,130,366; 5,310,667; 4,535,060; 4,769,061; 5,633,448; 5,510,471; RE 36,449; RE 37,287 E; and 5,491,288; and international publications WO 97/04103; WO 00/66746; WO 01/66704; and WO 00/66747, 6,040,497; 5,094,945; 5,554,798; 6,040,497; Zhou et al. (1995) Plant Cell Rep.: 159-163; WO 0234946; WO 9204449; 6,225,112; 4,535,060, and 6,040,497, which are incorporated herein by reference in their entireties for all purposes. Additional EPSP synthase sequences include, gdc-1 (U.S. App. Publication 20040205847); EPSP synthases with class III domains (U.S. App. Publication 20060253921); gdc-1 (U.S. App. Publication 20060021093); gdc-2 (U.S. App. Publication 20060021094); gro-1 (U.S. App. Publication 20060150269); grg23 or grg 51 (U.S. App. Publication 20070136840); GRG32 (U.S. App. Publication 20070300325); GRG33, GRG35, GRG36, GRG37, GRG38, GRG39 and GRG50 (U.S. App. Publication 20070300326); or EPSP synthase sequences disclosed in, U.S. App. Publication 20040177399; 20050204436; 20060150270; 20070004907; 20070044175; 2007010707; 20070169218; 20070289035; and, 20070295251; each of which is herein incorporated by reference in their entirety.

In other embodiments, the host cell, plant or plant cell or plant part having the heterologous polynucleotide encoding the GH3 polypeptide or an active variant or fragment thereof is stacked with, for example, a sequence which confers tolerance to an ALS inhibitor. As used herein, an “ALS inhibitor-tolerant polypeptide” comprises any polypeptide which when expressed in a plant confers tolerance to at least one ALS inhibitor. Varieties of ALS inhibitors are known and include, for example, sulfonylurea, imidazolinone, triazolopyrimidines, pryimidinyoxy(thio)benzoates, and/or sulfonylaminocarbonyltriazolinone herbicides. Additional ALS inhibitors are known and are disclosed elsewhere herein. It is known in the art that ALS mutations fall into different classes with regard to tolerance to sulfonylureas, imidazolinones, triazolopyrimidines, and pyrimidinyl(thio)benzoates, including mutations having the following characteristics: (1) broad tolerance to all four of these groups; (2) tolerance to imidazolinones and pyrimidinyl(thio)benzoates; (3) tolerance to sulfonylureas and triazolopyrimidines; and (4) tolerance to sulfonylureas and imidazolinones.

Various ALS inhibitor-tolerant polypeptides can be employed. In some embodiments, the ALS inhibitor-tolerant polynucleotides contain at least one nucleotide mutation resulting in one amino acid change in the ALS polypeptide. In specific embodiments, the change occurs in one of seven substantially conserved regions of acetolactate synthase. See, for example, Hattori et al. (1995) Molecular Genetics and Genomes 246:419-425; Lee et al. (1998) EMBO Journal 7:1241-1248; Mazur et al. (1989) Ann. Rev. Plant Phys. 40:441-470; and U.S. Pat. No. 5,605,011, each of which is incorporated by reference in their entirety. The ALS inhibitor-tolerant polypeptide can be encoded by, for example, the SuRA or SuRB locus of ALS. In specific embodiments, the ALS inhibitor-tolerant polypeptide comprises the C3 ALS mutant, the HRA ALS mutant, the S4 mutant or the S4/HRA mutant or any combination thereof. Different mutations in ALS are known to confer tolerance to different herbicides and groups (and/or subgroups) of herbicides; see, e.g., Tranel and Wright (2002) Weed Science 50:700-712. See also, U.S. Pat. Nos. 5,605,011, 5,378,824, 5,141,870, and 5,013,659, each of which is herein incorporated by reference in their entirety. The soybean, maize, and Arabidopsis HRA sequences are disclosed, for example, in WO2007/024782, herein incorporated by reference.

In some embodiments, the ALS inhibitor-tolerant polypeptide confers tolerance to sulfonylurea and imidazolinone herbicides. The production of sulfonylurea-tolerant plants and imidazolinone-tolerant plants is described more fully in U.S. Pat. Nos. 5,605,011; 5,013,659; 5,141,870; 5,767,361; 5,731,180; 5,304,732; 4,761,373; 5,331,107; 5,928,937; and 5,378,824; and international publication WO 96/33270, which are incorporated herein by reference in their entireties for all purposes. In specific embodiments, the ALS inhibitor-tolerant polypeptide comprises a sulfonamide-tolerant acetolactate synthase (otherwise known as a sulfonamide-tolerant acetohydroxy acid synthase) or an imidazolinone-tolerant acetolactate synthase (otherwise known as an imidazolinone-tolerant acetohydroxy acid synthase).

In further embodiments, the host cell, plants or plant cell or plant part having the heterologous polynucleotide encoding the GH3 polypeptide or an active variant or fragment thereof is stacked with, for example, a sequence which confers tolerance to an ALS inhibitor and glyphosate tolerance. In one embodiment, the polynucleotide encoding the GH3 polypeptide or active variant or fragment thereof is stacked with HRA and a glyphosate N-acetyltransferase. See, WO2007/024782, 2008/0051288 and WO 2008/112019, each of which is herein incorporated by reference.

Other examples of herbicide-tolerance traits that could be combined with the host cell, plant or plant cell or plant part having the heterologous polynucleotide encoding the GH3 polypeptide or an active variant or fragment thereof include those conferred by polynucleotides encoding an exogenous phosphinothricin acetyltransferase, as described in U.S. Pat. Nos. 5,969,213; 5,489,520; 5,550,318; 5,874,265; 5,919,675; 5,561,236; 5,648,477; 5,646,024; 6,177,616; and 5,879,903. Plants containing an exogenous phosphinothricin acetyltransferase can exhibit improved tolerance to glufosinate herbicides, which inhibit the enzyme glutamine synthase. Other examples of herbicide-tolerance traits that could be combined with the plants or plant cell or plant part having the heterologous polynucleotide encoding the GH3 polypeptide or an active variant or fragment thereof include those conferred by polynucleotides conferring altered protoporphyrinogen oxidase (protox) activity, as described in U.S. Pat. Nos. 6,288,306 B1; 6,282,837 B1; and 5,767,373; and international publication WO 01/12825. Plants containing such polynucleotides can exhibit improved tolerance to any of a variety of herbicides which target the protox enzyme (also referred to as “protox inhibitors”).

Other examples of herbicide-tolerance traits that could be combined with the host cell, plant or plant cell or plant part having the heterologous polynucleotide encoding the GH3 polypeptide or an active variant or fragment thereof include those conferring tolerance to at least one herbicide in a plant such as, for example, a maize plant or horseweed. Herbicide-tolerant weeds are known in the art, as are plants that vary in their tolerance to particular herbicides. See, e.g., Green and Williams (2004) “Correlation of Corn (Zea mays) Inbred Response to Nicosulfuron and Mesotrione,” poster presented at the WSSA Annual Meeting in Kansas City, Mo., Feb. 9-12, 2004; Green (1998) Weed Technology 12: 474-477; Green and Ulrich (1993) Weed Science 41: 508-516. The trait(s) responsible for these tolerances can be combined by breeding or via other methods with the plants or plant cell or plant part having the heterologous polynucleotide encoding the GH3 or an active variant or fragment thereof to provide a plant of the invention, as well as, methods of use thereof.

In still further embodiments, the polynucleotide encoding the GH3 polypeptide can be stacked with at least one polynucleotide encoding a homogentisate solanesyltransferase (HST). See, for example, WO2010/023911 herein incorporated by reference in its entirety. In such embodiments, classes of herbicidal compounds—which act wholly or in part by inhibiting HST can be applied over the plants having the HTS polypeptide.

The host cell, plant or plant cell or plant part having the polynucleotide encoding the GH3 polypeptide or an active variant or fragment thereof can also be combined with at least one other trait to produce plants that further comprise a variety of desired trait combinations including, but not limited to, traits desirable for animal feed such as high oil content (e.g., U.S. Pat. No. 6,232,529); balanced amino acid content (e.g., hordothionins (U.S. Pat. Nos. 5,990,389; 5,885,801; 5,885,802; and 5,703,409; U.S. Pat. No. 5,850,016); barley high lysine (Williamson et al. (1987) Eur. J. Biochem. 165: 99-106; and WO 98/20122) and high methionine proteins (Pedersen et al. (1986) J. Biol. Chem. 261: 6279; Kirihara et al. (1988) Gene 71: 359; and Musumura et al. (1989) Plant Mol. Biol. 12:123)); increased digestibility (e.g., modified storage proteins (U.S. application Ser. No. 10/053,410, filed Nov. 7, 2001); and thioredoxins (U.S. application Ser. No. 10/005,429, filed Dec. 3, 2001)); the disclosures of which are herein incorporated by reference. Desired trait combinations also include LLNC (low linolenic acid content; see, e.g., Dyer et al. (2002) Appl. Microbiol. Biotechnol. 59: 224-230) and OLCH (high oleic acid content; see, e.g., Fernandez-Moya et al. (2005) J. Agric. Food Chem. 53: 5326-5330).

The host cell, plant or plant cell or plant part having the polynucleotide encoding the GH3 polypeptide or an active variant or fragment thereof can also be combined with other desirable traits such as, for example, fumonisim detoxification genes (U.S. Pat. No. 5,792,931), avirulence and disease resistance genes (Jones et al. (1994) Science 266: 789; Martin et al. (1993) Science 262: 1432; Mindrinos et al. (1994) Cell 78: 1089), and traits desirable for processing or process products such as modified oils (e.g., fatty acid desaturase genes (U.S. Pat. No. 5,952,544; WO 94/11516)); modified starches (e.g., ADPG pyrophosphorylases (AGPase), starch synthases (SS), starch branching enzymes (SBE), and starch debranching enzymes (SDBE)); and polymers or bioplastics (e.g., U.S. Pat. No. 5,602,321; beta-ketothiolase, polyhydroxybutyrate synthase, and acetoacetyl-CoA reductase (Schubert et al. (1988) J. Bacteriol. 170:5837-5847) facilitate expression of polyhydroxyalkanoates (PHAs)); the disclosures of which are herein incorporated by reference. One could also combine herbicide-tolerant polynucleotides with polynucleotides providing agronomic traits such as male sterility (e.g., see U.S. Pat. No. 5,583,210), stalk strength, flowering time, or transformation technology traits such as cell cycle regulation or gene targeting (e.g., WO 99/61619, WO 00/17364, and WO 99/25821); the disclosures of which are herein incorporated by reference.

In other embodiments, the host cell, plant or plant cell or plant part having the polynucleotide encoding the GH3 polypeptide or an active variant or fragment thereof may be stacked with any other polynucleotides encoding polypeptides having pesticidal and/or insecticidal activity, such as Bacillus thuringiensis toxic proteins (described in U.S. Pat. Nos. 5,366,892; 5,747,450; 5,737,514; 5,723,756; 5,593,881; Geiser et al. (1986) Gene 48: 109; Lee et al. (2003) Appl. Environ. Microbiol. 69: 4648-4657 (Vip3A); Galitzky et al. (2001) Acta Crystallogr. D. Biol. Crystallogr. 57: 1101-1109 (Cry3Bb1); and Herman et al. (2004) J. Agric. Food Chem. 52: 2726-2734 (CrylF)); lectins (Van Damme et al. (1994) Plant Mol. Biol. 24: 825, pentin (described in U.S. Pat. No. 5,981,722), and the like. The combinations generated can also include multiple copies of any one of the polynucleotides of interest.

In another embodiment, the host cell, plant or plant cell or plant part having the polynucleotide encoding the GH3 polypeptide or an active variant or fragment thereof can also be combined with the Rcg1 sequence or biologically active variant or fragment thereof. The Rcg1 sequence is an anthracnose stalk rot resistance gene in corn. See, for example, U.S. patent application Ser. Nos. 11/397,153, 11/397,275, and 11/397,247, each of which is herein incorporated by reference.

These stacked combinations can be created by any method including, but not limited to, breeding plants by any conventional methodology, or genetic transformation. If the sequences are stacked by genetically transforming the plants, the polynucleotide sequences of interest can be combined at any time and in any order. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO99/25821, WO99/25854, WO99/25840, WO99/25855, and WO99/25853, all of which are herein incorporated by reference. Additional systems can be used for site specific integration including, for example, various meganucleases systems as set forth in WO 2009/114321 (herein incorporated by reference), which describes “custom” meganucleases. See, also, Gao et al. (2010) Plant Journal 1:176-187. Additional site specific integration systems include, but are not limited, to Zn Fingers, meganucleases, and TAL nucleases. See, for example, WO2010/079430, WO2011/072246, and US20110201118, each of which is herein incorporated by reference in their entirety.

### VI. Method of Introducing

Various methods can be used to introduce a sequence of interest into a host cell, plant or plant part. “Introducing” is intended to mean presenting to the host cell, plant, plant cell or plant part the polynucleotide or polypeptide in such a manner that the sequence gains access to the interior of a cell. The methods disclosed herein do not depend on a particular method for introducing a sequence into a host cell, plant or plant part, only that the polynucleotide or polypeptides gains access to the interior of at least one cell. Methods for introducing polynucleotides or polypeptides into plants are known in the art including, but not limited to, stable transformation methods, transient transformation methods, and virus-mediated methods.

“Stable transformation” is intended to mean that the nucleotide construct introduced into a host cell or plant integrates into the genome of the host cell or plant and is capable of being inherited by the progeny thereof “Transient transformation” is intended to mean that a polynucleotide is introduced into the host cell or plant and does not integrate into the genome of the host cell or plant or a polypeptide is introduced into a host cell or plant.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. No. 5,563,055 and U.S. Pat. No. 5,981,840), direct gene transfer (Paszkowski et al. (1984) EMBO J. 3:2717-2722), and ballistic particle acceleration (see, for example, U.S. Pat. No. 4,945,050; U.S. Pat. No. 5,879,918; U.S. Pat. Nos. 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

In specific embodiments, the GH3 sequences or active variant or fragments thereof can be provided to a plant using a variety of transient transformation methods. Such transient transformation methods include, but are not limited to, the introduction of the GH3 protein or active variants and fragments thereof directly into the plant. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) Proc. Natl. Acad. Sci. 91: 2176-2180 and Hush et al. (1994) The Journal of Cell Science 107:775-784, all of which are herein incorporated by reference.

In other embodiments, the polynucleotide encoding the GH3 polypeptide or active variants or fragments thereof may be introduced into plants by contacting plants with a virus or viral nucleic acids. Generally, such methods involve incorporating a nucleotide construct of the invention within a DNA or RNA molecule. It is recognized that the an GH3 sequence may be initially synthesized as part of a viral polyprotein, which later may be processed by proteolysis in vivo or in vitro to produce the desired recombinant protein. Further, it is recognized that promoters of the invention also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855, and WO99/25853, all of which are herein incorporated by reference. Briefly, the polynucleotide of the invention can be contained in transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided and the transfer cassette is integrated at the target site. The polynucleotide of interest is thereby integrated at a specific chromosomal position in the plant genome. Other methods to target polynucleotides are set forth in WO 2009/114321 (herein incorporated by reference), which describes “custom” meganucleases produced to modify plant genomes, in particular the genome of maize. See, also, Gao et al. (2010) Plant Journal 1:176-187.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a polynucleotide of the invention, for example, an expression cassette of the invention, stably incorporated into their genome.

Additional host cells of interest include, for example, prokaryotes including various strains of E. coli and other microbial strains. Commonly used prokaryotic control sequences which are defined herein to include promoters for transcription initiation, optionally with an operator, along with ribosome binding sequences, include such commonly used promoters as the beta lactamase (penicillinase) and lactose (lac) promoter systems (Chang et al. (1977) Nature 198:1056), the tryptophan (trp) promoter system (Goeddel et al. (1980) Nucleic Acids Res. 8:4057) and the lambda derived P L promoter and N-gene ribosome binding site (Shimatake et al. (1981) Nature 292:128). The inclusion of selection markers in DNA vectors transfected in E coli. is also useful. Examples of such markers include genes specifying resistance to ampicillin, tetracycline, or chloramphenicol.

The vector is selected to allow introduction into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing a protein of the present invention are available using Bacillus sp. and Salmonella (Palva et al. (1983) Gene 22:229-235); Mosbach et al. (1983) Nature 302:543-545).

A variety of expression systems for yeast are known to those of skill in the art. Two widely utilized yeasts for production of eukaryotic proteins are Saccharomyces cerevisiae and Pichia pastoris. Vectors, strains, and protocols for expression in Saccharomyces and Pichia are known in the art and available from commercial suppliers. See, for Example, Sherman et al. (1982) Methods in Yeast Genetics, Cold Spring Harbor Laboratory.

### VII. Methods of Use

A. Methods to Detoxify an Auxin-Analog Herbicide

A method for detoxifying an auxin-analog herbicide is provided. Such methods employ increasing the level of a GH3 polypeptide or an active variant or fragment thereof in a plant, plant cell, plant part, explant, seed and applying to the plant, plant cell or plant part at least one auxin-analog herbicide. The GH3 polypeptide has amino acid/auxin-analog herbicide conjugation activity and can act to form an amino acid/auxin conjugate having reduced herbicide activity. In specific embodiments, the amino acid/auxin conjugated product having reduced herbicidal activity comprises an aspartate/auxin-analog conjugate, a glutamate/auxin-analog conjugate, an aspartate/dicamba conjugate, a glutamate/dicamba conjugate, an aspartate/2,4-D conjugate, and/or a glutamate/2,4-D conjugate.

In further embodiments, the concentration/level of the GH3 polypeptide is increased in a host cell, plant or plant part by at least 10%, 20%, 30%, 40%, 50%, 60%, 70%, 80%, 90%, 100%, 200%, 500%, 1000%, 5000%, or 10,000% relative to an appropriate control plant, plant part, or cell which did not have or did not express the heterologous GH3 sequence. In still other embodiments, the level of the GH3 polypeptide in the plant or plant part is increased by 10, 20, 30, 40, 50, 60, 70, 80, 90, 100 fold or more compared to the level of the native GH3 sequence. Such an increase in the level of the GH3 polypeptide can be achieved in a variety of ways including, for example, by the expression of multiple copies of one or more GH3 polypeptide and/or by employing a promoter to drive higher levels of expression of the sequence.

In specific embodiments, the polynucleotide encoding the GH3 polypeptide or active variant or fragment thereof is introduced into the host cell, plant, plant cell, explant or plant part. Subsequently, a host cell or plant cell having the introduced GH3 sequence is selected using methods known to those of skill in the art such as, but not limited to, Southern blot analysis, DNA sequencing, PCR analysis, or phenotypic analysis. A plant or plant part altered or modified by the foregoing embodiments is grown under plant forming conditions for a time sufficient to modulate the concentration and/or activity of polypeptides of the present invention in the plant. Plant forming conditions are well known in the art and discussed briefly elsewhere herein.

In one embodiment, a method of producing an auxin-analog herbicide tolerant plant cell is provided and comprises transforming a plant cell with the polynucleotide encoding a GH3 polypeptide or an active variant or fragment thereof. In specific embodiments, the method further comprises selecting a plant cell which shows an increased resistance or tolerance to an auxin-analog herbicide (such as, for example, dicamba or 2,4-D) by growing the plant cells in a sufficient concentration of the auxin-analog herbicide and selecting cells or plants which show an increased tolerance to the auxin-analog herbicide.

It is also recognized that the level and/or activity of the native GH3 sequence in a plant or plant cell may be altered by employing a polynucleotide that is not capable of directing, in a transformed plant, the expression of a protein or an RNA. For example, the polynucleotide encoding the GH3 polypeptide or active variant or fragment thereof may be used to design polynucleotide constructs that can be employed in methods for altering or mutating a genomic nucleotide sequence in an organism. Such polynucleotide constructs include, but are not limited to, RNA:DNA vectors, RNA:DNA mutational vectors, RNA:DNA repair vectors, mixed-duplex oligonucleotides, self-complementary RNA:DNA oligonucleotides, and recombinogenic oligonucleobases. Such nucleotide constructs and methods of use are known in the art. See, U.S. Pat. Nos. 5,565,350; 5,731,181; 5,756,325; 5,760,012; 5,795,972; and 5,871,984; all of which are herein incorporated by reference. See also, WO 98/49350, WO 99/07865, WO 99/25821, and Beetham et al. (1999) Proc. Natl. Acad. Sci. USA 96:8774-8778; herein incorporated by reference.

It is therefore recognized that methods of the present invention do not depend on the incorporation of the entire polynucleotide into the genome, only that the plant or cell thereof is altered as a result of the introduction of the polynucleotide into a cell. In one embodiment, the genome may be altered following the introduction of the polynucleotide into a cell. Alterations to the genome include, but are not limited to, additions, deletions, and substitutions of nucleotides into the genome. While the methods provided herein do not depend on additions, deletions, and substitutions of any particular number of nucleotides, it is recognized that such additions, deletions, or substitutions comprises at least one nucleotide.

In another embodiment, a method of producing an auxin-analog herbicide tolerant host cell (i.e., a microbial cell such as E. coli) is provided and comprises introducing into the host cell (i.e., the microbial cell, such as E. coli) a polynucleotide encoding a GH3 polypeptide or an active variant or fragment thereof. Microbial host cells expressing such GH3 sequences find use in bioremediation.

As used herein, “bioremediation” is the use of micro-organism metabolism to remove a contaminating material. In such embodiments, an effective amount of the microbial host expressing the GH3 polypeptide is contacted with a contaminated material (i.e., soil) having an auxin-analog herbicide (such as, for example, dicamba or 2,4-D). The microbial host detoxifies the auxin-analog herbicide and thereby reduces the level of the contaminant in the material (i.e., soil). Such methods can occur either in situ or ex situ. In situ bioremediation involves treating the contaminated material at the site, while ex situ involves the removal of the contaminated material to be treated elsewhere.

B. Method of Producing Crops and Controlling Weeds

Methods for controlling weeds in an area of cultivation, preventing the development or the appearance of herbicide resistant weeds in an area of cultivation, producing a crop, and increasing crop safety are provided. The term “controlling,” and derivations thereof, for example, as in “controlling weeds” refers to one or more of inhibiting the growth, germination, reproduction, and/or proliferation of; and/or killing, removing, destroying, or otherwise diminishing the occurrence and/or activity of a weed.

As used herein, an “area of cultivation” comprises any region in which one desires to grow a plant. Such areas of cultivations include, but are not limited to, a field in which a plant is cultivated (such as a crop field, a sod field, a tree field, a managed forest, a field for culturing fruits and vegetables, etc.), a greenhouse, a growth chamber, etc.

As used herein, by “selectively controlled” it is intended that the majority of weeds in an area of cultivation are significantly damaged or killed, while if crop plants are also present in the field, the majority of the crop plants are not significantly damaged. Thus, a method is considered to selectively control weeds when at least 55%, 60%, 65%, 70%, 75%, 80%, 85%, 90%, 95%, or more of the weeds are significantly damaged or killed, while if crop plants are also present in the field, less than 45%, 40%, 35%, 30%, 25%, 20%, 15%, 10%, 5%, or 1% of the crop plants are significantly damaged or killed.

Methods provided comprise planting the area of cultivation with a plant or a seed having a heterologous polynucleotide encoding a GH3 polypeptide or an active variant or fragment thereof, and in specific embodiments, applying to the crop, seed, weed and/or area of cultivation thereof an effective amount of a herbicide of interest. It is recognized that the herbicide can be applied before or after the crop is planted in the area of cultivation. Such herbicide applications can include an application of an auxin-analog herbicide including, but not limited to, an auxin-analog herbicides from one of the following families: phenoxy, carboxylic acid (or pyridine), benzoic acid, aminocyclopyrachlor, and quinaline carboxylic acid. In specific embodiments, the auxin-analog herbicide can comprise at least one of (2,4-dichlorophenoxy)acetic acid, 4-(2,4-dichlorophenoxy)butyric acid (2,4-DB), 2-(2,4-dichlorophenoxy)propanoic acid (2,4-DP), (2,4,5-trichlorophenoxy)acetic acid (2,4,5-T), 2-(2,4,5-Trichlorophenoxyl)Propionic Acid (2,4,5-TP), 2-(2,4-dichloro-3-methylphenoxy)-N-phenylpropanamide (clomeprop), (4-chloro-2-methylphenoxy)acetic acid (MCPA), 4-(4-chloro-o-tolyloxy)butyric acid (MCPB), 2-(4-chloro-2-methylphenoxy)propanoic acid (MCPP), 3,6-dichloro-2-pyridinecarboxylic acid (Clopyralid), 4-amino-3,5,6-trichloro-2-pyridinecarboxylic acid (picloram), (2,4,5-trichlorophenoxy) acetic acid (triclopyr), 4-amino-3,5-dichloro-6-fluoro-2-pyridyloxyacetic acid (fluoroxypyr), 3,6-dichloro-o-anisic acid (dicamba) and 3-amino-2,5-dichlorobenzoic acid (choramben), 3,7-dichloro-8-quinolinecarboxylic acid (quinclorac) and 7-chloro-3-methyl-8-quinolinecarboxylic acid (quinmerac). In specific embodiments, the auxin-analog herbicide comprises dicamba or 2,4-D. Generally, the effective amount of herbicide applied to the field is sufficient to selectively control the weeds without significantly affecting the crop.

“Weed” as used herein refers to a plant which is not desirable in a particular area. Conversely, a “crop plant” as used herein refers to a plant which is desired in a particular area, such as, for example, a maize or soybean plant. Thus, in some embodiments, a weed is a non-crop plant or a non-crop species, while in some embodiments, a weed is a crop species which is sought to be eliminated from a particular area, such as, for example, an inferior and/or non-transgenic soybean plant in a field planted with a plant having the heterologous nucleotide sequence encoding the GH3 polypeptide or an active variant or fragment thereof

Further provided is a method for producing a crop by growing a crop plant that is tolerant to an auxin-analog herbicide as a result of being transformed with a heterologous polynucleotide encoding a GH3 polypeptide or an active variant or fragment thereof, under conditions such that the crop plant produces a crop, and harvesting the crop. Preferably, an auxin-analog herbicide is applied to the plant, or in the vicinity of the plant, or in the area of cultivation at a concentration effective to control weeds without preventing the transgenic crop plant from growing and producing the crop. The application of the auxin-analog herbicide can be before planting, or at any time after planting up to and including the time of harvest. The auxin-analog herbicide can be applied once or multiple times. The timing of the auxin-analog herbicide application, amount applied, mode of application, and other parameters will vary based upon the specific nature of the crop plant and the growing environment. The invention further provides the crop produced by this method.

Further provided are methods for the propagation of a plant containing a heterologous polynucleotide encoding a GH3 polypeptide or active variant or fragment thereof. The plant can be, for example, a monocot or a dicot. In one aspect, propagation entails crossing a plant containing the heterologous polynucleotide encoding a GH3 polypeptide transgene with a second plant, such that at least some progeny of the cross display auxin-analog herbicide tolerance.

The methods of the invention further allow for the development of herbicide applications to be used with the plants having the heterologous polynucleotides encoding the GH3 polypeptides or active variants or fragments thereof. In such methods, the environmental conditions in an area of cultivation are evaluated. Environmental conditions that can be evaluated include, but are not limited to, ground and surface water pollution concerns, intended use of the crop, crop tolerance, soil residuals, weeds present in area of cultivation, soil texture, pH of soil, amount of organic matter in soil, application equipment, and tillage practices. Upon the evaluation of the environmental conditions, an effective amount of a combination of herbicides can be applied to the crop, crop part, seed of the crop or area of cultivation.

Any herbicide or combination of herbicides can be applied to the plant having the heterologous polynucleotide encoding the GH3 polypeptide or active variant or fragment thereof disclosed herein or transgenic seed derived there from, crop part, or the area of cultivation containing the crop plant. By “treated with a combination of” or “applying a combination of” herbicides to a crop, area of cultivation or field it is intended that a particular field, crop or weed is treated with each of the herbicides and/or chemicals indicated to be part of the combination so that a desired effect is achieved, i.e., so that weeds are selectively controlled while the crop is not significantly damaged. The application of each herbicide and/or chemical may be simultaneous or the applications may be at different times (sequential), so long as the desired effect is achieved. Furthermore, the application can occur prior to the planting of the crop.

Classifications of herbicides (i.e., the grouping of herbicides into classes and subclasses) are well-known in the art and include classifications by HRAC (Herbicide Resistance Action Committee) and WSSA (the Weed Science Society of America) (see also, Retzinger and Mallory-Smith (1997) Weed Technology 11: 384-393). An abbreviated version of the HRAC classification (with notes regarding the corresponding WSSA group) is set forth below in Table 1.

Herbicides can be classified by their mode of action and/or site of action and can also be classified by the time at which they are applied (e.g., preemergent or postemergent), by the method of application (e.g., foliar application or soil application), or by how they are taken up by or affect the plant or by their structure. “Mode of action” generally refers to the metabolic or physiological process within the plant that the herbicide inhibits or otherwise impairs, whereas “site of action” generally refers to the physical location or biochemical site within the plant where the herbicide acts or directly interacts. Herbicides can be classified in various ways, including by mode of action and/or site of action (see, e.g., Table 1).

In specific embodiments, the plants of the present invention can tolerate treatment with different types of herbicides (i.e., herbicides having different modes of action and/or different sites of action) thereby permitting improved weed management strategies that are recommended in order to reduce the incidence and prevalence of herbicide-tolerant weeds.

TABLE 1
Abbreviated version of HRAC Herbicide Classification.

I. ALS Inhibitors (WSSA Group 2)

A. Sulfonylureas

```
  1. Azimsulfuron
  2. Chlorimuron-ethyl
  3. Metsulfuron-methyl
  4. Nicosulfuron
  5. Rimsulfuron
  6. Sulfometuron-methyl
  7. Thifensulfuron-methyl
  8. Tribenuron-methyl
  9. Amidosulfuron
10. Bensulfuron-methyl
11. Chlorsulfuron
12. Cinosulfuron
13. Cyclosulfamuron
14. Ethametsulfuron-methyl
15. Ethoxysulfuron
16. Flazasulfuron
17. Flupyrsulfuron-methyl
18. Foramsulfuron
19. Imazosulfuron
20. Iodosulfuron-methyl
21. Mesosulfuron-methyl
22. Oxasulfuron
23. Primisulfuron-methyl
24. Prosulfuron
25. Pyrazosulfuron-ethyl
26. Sulfosulfuron
27. Triasulfuron
28. Trifloxysulfuron
29. Triflusulfuron-methyl
30. Tritosulfuron
31. Halosulfuron-methyl
32. Flucetosulfuron
```

B. Sulfonylaminocarbonyltriazolinones

1. Flucarbazone
2. Procarbazone

C. Triazolopyrimidines

1. Cloransulam-methyl
2. Flumetsulam
3. Diclosulam
4. Florasulam
5. Metosulam
6. Penoxsulam
7. Pyroxsulam

D. Pyrimidinyloxy(thio)benzoates

1. Bispyribac
2. Pyriftalid
3. Pyribenzoxim
4. Pyrithiobac
5. Pyriminobac-methyl

E. Imidazolinones

1. Imazapyr
2. Imazethapyr
3. Imazaquin
4. Imazapic
5. Imazamethabenz-methyl
6. Imazamox

II. Other Herbicides--Active Ingredients/
Additional Modes of Action

A. Inhibitors of Acetyl CoA carboxylase

(ACCase) (WSSA Group 1)

1. Aryloxyphenoxypropionates (‘FOPs’)

a. Quizalofop-P-ethyl
b. Diclofop-methyl
c. Clodinafop-propargyl
d. Fenoxaprop-P-ethyl
e. Fluazifop-P-butyl
f. Propaquizafop
g. Haloxyfop-P-methyl
h. Cyhalofop-butyl
i. Quizalofop-P-ethyl

2. Cyclohexanediones (‘DIMs’)

a. Alloxydim
b. Butroxydim
c. Clethodim
d. Cycloxydim
e. Sethoxydim
f. Tepraloxydim
g. Tralkoxydim

B. Inhibitors of Photosystem II-HRAC

Group C1/WSSA Group 5

1. Triazines

a. Ametryne
b. Atrazine
c. Cyanazine
d. Desmetryne
e. Dimethametryne
f. Prometon
g. Prometryne
h. Propazine
i. Simazine
j. Simetryne
k. Terbumeton
l. Terbuthylazine
m. Terbutryne
n. Trietazine

2. Triazinones

a. Hexazinone
b. Metribuzin
c. Metamitron

3. Triazolinone

a. Amicarbazone

4. Uracils

a. Bromacil
b. Lenacil
c. Terbacil

5. Pyridazinones

a. Pyrazon

6. Phenyl carbamates

a. Desmedipham
b. Phenmedipham

C. Inhibitors of Photosystem II--HRAC

Group C2/WSSA Group 7

1. Ureas

a. Fluometuron
b. Linuron
c. Chlorobromuron
d. Chlorotoluron
e. Chloroxuron
f. Dimefuron
g. Diuron
h. Ethidimuron
i. Fenuron
j. Isoproturon
k. Isouron
l. Methabenzthiazuron
m. Metobromuron
n. Metoxuron
o. Monolinuron
p. Neburon
q. Siduron
r. Tebuthiuron

2. Amides

a. Propanil
b. Pentanochlor

D. Inhibitors of Photosystem II--HRAC

Group C3/WSSA Group 6

1. Nitriles

a. Bromofenoxim
b. Bromoxynil
c. Ioxynil

2. Benzothiadiazinone (Bentazon)

a. Bentazon

3. Phenylpyridazines

a. Pyridate
b. Pyridafol

E. Photosystem-I-electron diversion

(Bipyridyliums) (WSSA Group 22)

1. Diquat
2. Paraquat

F. Inhibitors of PPO (protoporphyrinogen

oxidase) (WSSA Group 14)

1. Diphenylethers

a. Acifluorfen-Na
b. Bifenox
c. Chlomethoxyfen
d. Fluoroglycofen-ethyl
e. Fomesafen
f. Halosafen
g. Lactofen
h. Oxyfluorfen

2. Phenylpyrazoles

a. Fluazolate
b. Pyraflufen-ethyl

3. N-phenylphthalimides

a. Cinidon-ethyl
b. Flumioxazin
c. Flumiclorac-pentyl

4. Thiadiazoles

a. Fluthiacet-methyl
b. Thidiazimin

5. Oxadiazoles

a. Oxadiazon
b. Oxadiargyl

6. Triazolinones

a. Carfentrazone-ethyl
b. Sulfentrazone

7. Oxazolidinediones

a. Pentoxazone

8. Pyrimidindiones

a. Benzfendizone
b. Butafenicil

9. Others

a. Pyrazogyl
b. Profluazol

G. Bleaching: Inhibition of carotenoid

biosynthesis at the phytoene desaturase step
(PDS) (WSSA Group 12)

1. Pyridazinones

a. Norflurazon

2. Pyridinecarboxamides

a. Diflufenican
b. Picolinafen

3. Others

a. Beflubutamid
b. Fluridone
c. Flurochloridone
d. Flurtamone

H. Bleaching: Inhibition of 4-

hydroxyphenyl-pyruvate-dioxygenase (4-HPPD)
(WSSA Group 28)

1. Triketones

a. Mesotrione
b. Sulcotrione
c. topramezone
d. tembotrione

2. Isoxazoles

a. Pyrasulfotole
b. Isoxaflutole

3. Pyrazoles

a. Benzofenap
b. Pyrazoxyfen
c. Pyrazolynate

4. Others

a. Benzobicyclon

I. Bleaching: Inhibition of carotenoid

biosynthesis (unknown target) (WSSA Group 11
and 13)

1. Triazoles (WSSA Group 11)

a. Amitrole

2. Isoxazolidinones (WSSA Group 13)

a. Clomazone

3. Ureas

a. Fluometuron

3. Diphenylether

a. Aclonifen

J. Inhibition of EPSP Synthase

1. Glycines (WSSA Group 9)

a. Glyphosate
b. Sulfosate

K. Inhibition of glutamine synthetase

1. Phosphinic Acids

a. Glufosinate-ammonium
b. Bialaphos

L. Inhibition of DHP (dihydropteroate)

synthase (WSSA Group 18)

1 Carbamates

a. Asulam

M. Microtubule Assembly Inhibition

(WSSA Group 3)

1. Dinitroanilines

a. Benfluralin
b. Butralin
c. Dinitramine
d. Ethalfluralin
e. Oryzalin
f. Pendimethalin
g. Trifluralin

2. Phosphoroamidates

a. Amiprophos-methyl
b. Butamiphos

3. Pyridines

a. Dithiopyr
b. Thiazopyr

4. Benzamides

a. Pronamide
b. Tebutam

5. Benzenedicarboxylic acids

a. Chlorthal-dimethyl

N. Inhibition of mitosis/microtubule

organization WSSA Group 23)

1. Carbamates

a. Chlorpropham
b. Propham
c. Carbetamide

O. Inhibition of cell division (Inhibition of

very long chain fatty acids as proposed
mechanism; WSSA Group 15)

1. Chloroacetamides

a. Acetochlor
b. Alachlor
c. Butachlor
d. Dimethachlor
e. Dimethanamid
f. Metazachlor
g. Metolachlor
h. Pethoxamid
i. Pretilachlor
j. Propachlor
k. Propisochlor
l. Thenylchlor

2. Acetamides

a. Diphenamid
b. Napropamide
c. Naproanilide

3. Oxyacetamides

a. Flufenacet
b. Mefenacet

4. Tetrazolinones

a. Fentrazamide

5. Others

a. Anilofos
b. Cafenstrole
c. Indanofan
d. Piperophos

P. Inhibition of cell wall (cellulose)

synthesis

1. Nitriles (WSSA Group 20)

a. Dichlobenil
b. Chlorthiamid

2. Benzamides (isoxaben (WSSA

Group 21))

a. Isoxaben

3. Triazolocarboxamides (flupoxam)

a. Flupoxam

Q. Uncoupling (membrane disruption):

(WSSA Group 24)

1. Dinitrophenols

a. DNOC
b. Dinoseb
c. Dinoterb

R. Inhibition of Lipid Synthesis by other

than ACC inhibition

1. Thiocarbamates (WSSA Group 8)

a. Butylate
b. Cycloate
c. Dimepiperate
d. EPTC
e. Esprocarb
f. Molinate
g. Orbencarb
h. Pebulate
i. Prosulfocarb
j. Benthiocarb
k. Tiocarbazil
l. Triallate
m. Vernolate

2. Phosphorodithioates

a. Bensulide

3. Benzofurans

a. Benfuresate
b. Ethofumesate

4. Halogenated alkanoic acids

(WSSA Group 26)

a. TCA
b. Dalapon
c. Flupropanate

S. Synthetic auxins (IAA-like) (WSSA

Group 4)

1. Phenoxycarboxylic acids

a. Clomeprop
b. 2,4-D
c. Mecoprop

2. Benzoic acids

a. Dicamba
b. Chloramben
c. TBA

3. Pyridine carboxylic acids

a. Clopyralid
b. Fluroxypyr
c. Picloram
d. Tricyclopyr

4. Quinoline carboxylic acids

a. Quinclorac
b. Quinmerac

5. Others (benazolin-ethyl)

a. Benazolin-ethyl

6. aminocyclopyrachlor

T. Inhibition of Auxin Transport

1. Phthalamates; semicarbazones

(WSSA Group 19)

a. Naptalam
b. Diflufenzopyr-Na

U. Other Mechanism of Action

1. Arylaminopropionic acids

a. Flamprop-M-methyl/-

isopropyl

2. Pyrazolium

a. Difenzoquat

3. Organoarsenicals

a. DSMA
b. MSMA

4. Others

a. Bromobutide
b. Cinmethylin
c. Cumyluron
d. Dazomet
e. Daimuron-methyl
f. Dimuron
g. Etobenzanid
h. Fosamine
i. Metam
j. Oxaziclomefone
k. Oleic acid
l. Pelargonic acid
m. Pyributicarb

In still further methods, an auxin-analog herbicide can be applied alone or in combination with another herbicide of interest and can be applied to the plants having the heterologous polynucleotide encoding the GH3 polypeptide or active variant or fragment thereof or their area of cultivation.

Additional herbicide treatment that can be applied over the plants or seeds having the heterologous polynucleotide encoding the GH3 polypeptide or an active variant or fragment thereof include, but are not limited to: acetochlor, acifluorfen and its sodium salt, aclonifen, acrolein (2-propenal), alachlor, alloxydim, ametryn, amicarbazone, amidosulfuron, aminopyralid, aminocyclopyrachlor, amitrole, ammonium sulfamate, anilofos, asulam, atrazine, azimsulfuron, beflubutamid, benazolin, benazolin-ethyl, bencarbazone, benfluralin, benfuresate, bensulfuron-methyl, bensulide, bentazone, benzobicyclon, benzofenap, bifenox, bilanafos, bispyribac and its sodium salt, bromacil, bromobutide, bromofenoxim, bromoxynil, bromoxynil octanoate, butachlor, butafenacil, butamifos, butralin, butroxydim, butylate, cafenstrole, carbetamide, carfentrazone-ethyl, catechin, chlomethoxyfen, chloramben, chlorbromuron, chlorflurenol-methyl, chloridazon, chlorimuron-ethyl, chlorotoluron, chlorpropham, chlorsulfuron, chlorthal-dimethyl, chlorthiamid, cinidon-ethyl, cinmethylin, cinosulfuron, clethodim, clodinafop-propargyl, clomazone, clomeprop, clopyralid, clopyralid-olamine, cloransulam-methyl, CUH-35 (2-methoxyethyl 2-[[[4-chloro-2-fluoro-5-[(1-methyl-2-propynyl)oxy]phenyl](3-fluorobenzoyl)amino]carbonyl]-1-cyclohexene-1-carboxylate), cumyluron, cyanazine, cycloate, cyclosulfamuron, cycloxydim, cyhalofop-butyl, 2,4-D and its butotyl, butyl, isoctyl and isopropyl esters and its dimethylammonium, diolamine and trolamine salts, daimuron, dalapon, dalapon-sodium, dazomet, 2,4-DB and its dimethylammonium, potassium and sodium salts, desmedipham, desmetryn, dicamba and its diglycolammonium, dimethylammonium, potassium and sodium salts, dichlobenil, dichlorprop, diclofop-methyl, diclosulam, difenzoquat metilsulfate, diflufenican, diflufenzopyr, dimefuron, dimepiperate, dimethachlor, dimethametryn, dimethenamid, dimethenamid-P, dimethipin, dimethylarsinic acid and its sodium salt, dinitramine, dinoterb, diphenamid, diquat dibromide, dithiopyr, diuron, DNOC, endothal, EPTC, esprocarb, ethalfluralin, ethametsulfuron-methyl, ethofumesate, ethoxyfen, ethoxysulfuron, etobenzanid, fenoxaprop-ethyl, fenoxaprop-P-ethyl, fentrazamide, fenuron, fenuron-TCA, flamprop-methyl, flamprop-M-isopropyl, flamprop-M-methyl, flazasulfuron, florasulam, fluazifop-butyl, fluazifop-P-butyl, flucarbazone, flucetosulfuron, fluchloralin, flufenacet, flufenpyr, flufenpyr-ethyl, flumetsulam, flumiclorac-pentyl, flumioxazin, fluometuron, fluoroglycofen-ethyl, flupyrsulfuron-methyl and its sodium salt, flurenol, flurenol-butyl, fluridone, flurochloridone, fluroxypyr, flurtamone, fluthiacet-methyl, fomesafen, foramsulfuron, fosamine-ammonium, glufosinate, glufosinate-ammonium, glyphosate and its salts such as ammonium, isopropylammonium, potassium, sodium (including sesquisodium) and trimesium (alternatively named sulfosate) (See, WO2007/024782, herein incorporated by reference), halosulfuron-methyl, haloxyfop-etotyl, haloxyfop-methyl, hexazinone, HOK-201 (N-(2,4-difluorophenyl)-1,5-dihydro-N-(1-methylethyl)-5-oxo-1-[(tetrahydro-2H-pyran-2-yl)methyl]-4H-1,2,4-triazole-4-carboxamide), imazamethabenz-methyl, imazamox, imazapic, imazapyr, imazaquin, imazaquin-ammonium, imazethapyr, imazethapyr-ammonium, imazosulfuron, indanofan, iodosulfuron-methyl, ioxynil, ioxynil octanoate, ioxynil-sodium, isoproturon, isouron, isoxaben, isoxaflutole, pyrasulfotole, lactofen, lenacil, linuron, maleic hydrazide, MCPA and its salts (e.g., MCPA-dimethylammonium, MCPA-potassium and MCPA-sodium, esters (e.g., MCPA-2-ethylhexyl, MCPA-butotyl) and thioesters (e.g., MCPA-thioethyl), MCPB and its salts (e.g., MCPB-sodium) and esters (e.g., MCPB-ethyl), mecoprop, mecoprop-P, mefenacet, mefluidide, mesosulfuron-methyl, mesotrione, metam-sodium, metamifop, metamitron, metazachlor, methabenzthiazuron, methylarsonic acid and its calcium, monoammonium, monosodium and disodium salts, methyldymron, metobenzuron, metobromuron, metolachlor, S-metholachlor, metosulam, metoxuron, metribuzin, metsulfuron-methyl, molinate, monolinuron, naproanilide, napropamide, naptalam, neburon, nicosulfuron, norflurazon, orbencarb, oryzalin, oxadiargyl, oxadiazon, oxasulfuron, oxaziclomefone, oxyfluorfen, paraquat dichloride, pebulate, pelargonic acid, pendimethalin, penoxsulam, pentanochlor, pentoxazone, perfluidone, pethoxyamid, phenmedipham, picloram, picloram-potassium, picolinafen, pinoxaden, piperofos, pretilachlor, primisulfuron-methyl, prodiamine, profoxydim, prometon, prometryn, propachlor, propanil, propaquizafop, propazine, propham, propisochlor, propoxycarbazone, propyzamide, prosulfocarb, prosulfuron, pyraclonil, pyraflufen-ethyl, pyrasulfotole, pyrazogyl, pyrazolynate, pyrazoxyfen, pyrazosulfuron-ethyl, pyribenzoxim, pyributicarb, pyridate, pyriftalid, pyriminobac-methyl, pyrimisulfan, pyrithiobac, pyrithiobac-sodium, pyroxsulam, quinclorac, quinmerac, quinoclamine, quizalofop-ethyl, quizalofop-P-ethyl, quizalofop-P-tefuryl, rimsulfuron, sethoxydim, siduron, simazine, simetryn, sulcotrione, sulfentrazone, sulfometuron-methyl, sulfosulfuron, 2,3,6-TBA, TCA, TCA-sodium, tebutam, tebuthiuron, tefuryltrione, tembotrione, tepraloxydim, terbacil, terbumeton, terbuthylazine, terbutryn, thenylchlor, thiazopyr, thiencarbazone, thifensulfuron-methyl, thiobencarb, tiocarbazil, topramezone, tralkoxydim, tri-allate, triasulfuron, triaziflam, tribenuron-methyl, triclopyr, triclopyr-butotyl, triclopyr-triethylammonium, tridiphane, trietazine, trifloxysulfuron, trifluralin, triflusulfuron-methyl, tritosulfuron and vernolate.

Additional herbicides include those that are applied over plants having homogentisate solanesyltransferase (HST) polypeptide such as those described in WO2010029311(A2), herein incorporate by reference it its entirety.

Other suitable herbicides and agricultural chemicals are known in the art, such as, for example, those described in WO 2005/041654. Other herbicides also include bioherbicides such as Alternaria destruens Simmons, Colletotrichum gloeosporiodes (Penz.) Penz. & Sacc., Drechsiera monoceras (MTB-951), Myrothecium verrucaria (Albertini & Schweinitz) Ditmar: Fries, Phytophthora palmivora (Butl.) Butl. and Puccinia thlaspeos Schub. Combinations of various herbicides can result in a greater-than-additive (i.e., synergistic) effect on weeds and/or a less-than-additive effect (i.e. safening) on crops or other desirable plants. In certain instances, combinations of auxin-analog herbicides with other herbicides having a similar spectrum of control but a different mode of action will be particularly advantageous for preventing the development of resistant weeds.

The time at which a herbicide is applied to an area of interest (and any plants therein) may be important in optimizing weed control. The time at which a herbicide is applied may be determined with reference to the size of plants and/or the stage of growth and/or development of plants in the area of interest, e.g., crop plants or weeds growing in the area.

Ranges of the effective amounts of herbicides can be found, for example, in various publications from University Extension services. See, for example, Bernards et al. (2006) Guide for Weed Management in Nebraska (www.ianrpubs.url.edu/sendlt/ec130); Regher et al. (2005) Chemical Weed Control for Fields Crops, Pastures, Rangeland, and Noncropland, Kansas State University Agricultural Extension Station and Corporate Extension Service; Zollinger et al. (2006) North Dakota Weed Control Guide, North Dakota Extension Service, and the Iowa State University Extension at www.weeds.iastate.edu, each of which is herein incorporated by reference.

Many plant species can be controlled (i.e., killed or damaged) by the herbicides described herein. Accordingly, the methods of the invention are useful in controlling these plant species where they are undesirable (i.e., where they are weeds). These plant species include crop plants as well as species commonly considered weeds, including but not limited to species such as: blackgrass (Alopecurus myosuroides), giant foxtail (Setaria faberi), large crabgrass (Digitaria sanguinalis), Surinam grass (Brachiaria decumbens), wild oat (Avena fatua), common cocklebur (Xanthium pensylvanicum), common lambsquarters (Chenopodium album), morning glory (Ipomoea coccinea), pigweed (Amaranthus spp.), common waterhemp (Amaranthus tuberculatus), velvetleaf (Abutilion theophrasti), common barnyardgrass (Echinochloa crus-galli), bermudagrass (Cynodon dactylon), downy brome (Bromus tectorum), goosegrass (Eleusine indica), green foxtail (Setaria viridis), Italian ryegrass (Lolium multiflorum), Johnsongrass (Sorghum halepense), lesser canarygrass (Phalaris minor), windgrass (Apera spica-venti), wooly cupgrass (Erichloa villosa), yellow nutsedge (Cyperus esculentus), common chickweed (Stellaria media), common ragweed (Ambrosia artemisiifolia), Kochia scoparia, horseweed (Conyza canadensis), rigid ryegrass (Lolium rigidum), goosegrass (Eleucine indica), hairy fleabane (Conyza bonariensis), buckhorn plantain (Plantago lanceolata), tropical spiderwort (Commelina benghalensis), field bindweed (Convolvulus arvensis), purple nutsedge (Cyperus rotundus), redvine (Brunnichia ovata), hemp sesbania (Sesbania exaltata), sicklepod (Senna obtusifolia), Texas blueweed (Helianthus ciliaris), and Devil's claws (Proboscidea louisianica). In other embodiments, the weed comprises a herbicide-resistant ryegrass, for example, a glyphosate resistant ryegrass, a paraquat resistant ryegrass, a ACCase-inhibitor resistant ryegrass, and a non-selective herbicide resistant ryegrass.

In some embodiments, a plant having the heterologous polynucleotide encoding the GH3 polypeptide or an active variant or fragment thereof is not significantly damaged by treatment with an auxin-analog herbicide applied to that plant, whereas an appropriate control plant is significantly damaged by the same treatment.

Generally, an auxin-analog herbicide is applied to a particular field (and any plants growing in it) no more than 1, 2, 3, 4, 5, 6, 7, or 8 times a year, or no more than 1, 2, 3, 4, or 5 times per growing season. Thus, methods of the invention encompass applications of herbicide which are “preemergent,” “postemergent,” “preplant incorporation” and/or which involve seed treatment prior to planting.

In one embodiment, methods are provided for coating seeds. The methods comprise coating a seed with an effective amount of a herbicide or a combination of herbicides (as disclosed elsewhere herein). The seeds can then be planted in an area of cultivation. Further provided are seeds having a coating comprising an effective amount of a herbicide or a combination of herbicides (as disclosed elsewhere herein). In other embodiments, the seeds can be coated with at least one fungicide and/or at least one insecticide and/or at least one herbicide or any combination thereof.

“Preemergent” refers to a herbicide which is applied to an area of interest (e.g., a field or area of cultivation) before a plant emerges visibly from the soil. “Postemergent” refers to a herbicide which is applied to an area after a plant emerges visibly from the soil. In some instances, the terms “preemergent” and “postemergent” are used with reference to a weed in an area of interest, and in some instances these terms are used with reference to a crop plant in an area of interest. When used with reference to a weed, these terms may apply to only a particular type of weed or species of weed that is present or believed to be present in the area of interest. While any herbicide may be applied in a preemergent and/or postemergent treatment, some herbicides are known to be more effective in controlling a weed or weeds when applied either preemergence or postemergence. For example, rimsulfuron has both preemergence and postemergence activity, while other herbicides have predominately preemergence (metolachlor) or postemergence (glyphosate) activity. These properties of particular herbicides are known in the art and are readily determined by one of skill in the art. Further, one of skill in the art would readily be able to select appropriate herbicides and application times for use with the transgenic plants of the invention and/or on areas in which transgenic plants of the invention are to be planted. “Preplant incorporation” involves the incorporation of compounds into the soil prior to planting.

Thus, improved methods of growing a crop and/or controlling weeds such as, for example, “pre-planting burn down,” are provided wherein an area is treated with herbicides prior to planting the crop of interest in order to better control weeds. The invention also provides methods of growing a crop and/or controlling weeds which are “no-till” or “low-till” (also referred to as “reduced tillage”). In such methods, the soil is not cultivated or is cultivated less frequently during the growing cycle in comparison to traditional methods; these methods can save costs that would otherwise be incurred due to additional cultivation, including labor and fuel costs.

The term “safener” refers to a substance that when added to a herbicide formulation eliminates or reduces the phytotoxic effects of the herbicide to certain crops. One of ordinary skill in the art would appreciate that the choice of safener depends, in part, on the crop plant of interest and the particular herbicide or combination of herbicides. Exemplary safeners suitable for use with the presently disclosed herbicide compositions include, but are not limited to, those disclosed in U.S. Pat. Nos. 4,808,208; 5,502,025; 6,124,240 and U.S. Patent Application Publication Nos. 2006/0148647; 2006/0030485; 2005/0233904; 2005/0049145; 2004/0224849; 2004/0224848; 2004/0224844; 2004/0157737; 2004/0018940; 2003/0171220; 2003/0130120; 2003/0078167, the disclosures of which are incorporated herein by reference in their entirety. The methods of the invention can involve the use of herbicides in combination with herbicide safeners such as benoxacor, BCS (1-bromo-4-[(chloromethyl) sulfonyl]benzene), cloquintocet-mexyl, cyometrinil, dichlormid, 2-(dichloromethyl)-2-methyl-1,3-dioxolane (MG 191), fenchlorazole-ethyl, fenclorim, flurazole, fluxofenim, furilazole, isoxadifen-ethyl, mefenpyr-diethyl, methoxyphenone ((4-methoxy-3-methylphenyl)(3-methylphenyl)-methanone), naphthalic anhydride (1,8-naphthalic anhydride) and oxabetrinil to increase crop safety. Antidotally effective amounts of the herbicide safeners can be applied at the same time as the compounds of this invention, or applied as seed treatments. Therefore an aspect of methods disclosed herein relates to the use of a mixture comprising an auxin-analog herbicide, at least one other herbicide, and an antidotally effective amount of a herbicide safener.

Seed treatment is useful for selective weed control, because it physically restricts antidoting to the crop plants. Therefore in one embodiment, a method for selectively controlling the growth of weeds in a field comprising treating the seed from which the crop is grown with an antidotally effective amount of safener and treating the field with an effective amount of herbicide to control weeds.

An antidotally effective amount of a safener is present where a desired plant is treated with the safener so that the effect of a herbicide on the plant is decreased in comparison to the effect of the herbicide on a plant that was not treated with the safener; generally, an antidotally effective amount of safener prevents damage or severe damage to the plant treated with the safener. One of skill in the art is capable of determining whether the use of a safener is appropriate and determining the dose at which a safener should be administered to a crop.

As used herein, an “adjuvant” is any material added to a spray solution or formulation to modify the action of an agricultural chemical or the physical properties of the spray solution. See, for example, Green and Foy (2003) “Adjuvants: Tools for Enhancing Herbicide Performance,” in Weed Biology and Management, ed. Inderjit (Kluwer Academic Publishers, The Netherlands). Adjuvants can be categorized or subclassified as activators, acidifiers, buffers, additives, adherents, antiflocculants, antifoamers, defoamers, antifreezes, attractants, basic blends, chelating agents, cleaners, colorants or dyes, compatibility agents, cosolvents, couplers, crop oil concentrates, deposition agents, detergents, dispersants, drift control agents, emulsifiers, evaporation reducers, extenders, fertilizers, foam markers, formulants, inerts, humectants, methylated seed oils, high load COCs, polymers, modified vegetable oils, penetrators, repellants, petroleum oil concentrates, preservatives, rainfast agents, retention aids, solubilizers, surfactants, spreaders, stickers, spreader stickers, synergists, thickeners, translocation aids, uv protectants, vegetable oils, water conditioners, and wetting agents.

In addition, methods of the invention can comprise the use of a herbicide or a mixture of herbicides, as well as, one or more other insecticides, fungicides, nematocides, bactericides, acaricides, growth regulators, chemosterilants, semiochemicals, repellents, attractants, pheromones, feeding stimulants or other biologically active compounds or entomopathogenic bacteria, virus, or fungi to form a multi-component mixture giving an even broader spectrum of agricultural protection. Examples of such agricultural protectants which can be used in methods of the invention include: insecticides such as abamectin, acephate, acetamiprid, amidoflumet (S-1955), avermectin, azadirachtin, azinphos-methyl, bifenthrin, bifenazate, buprofezin, carbofuran, cartap, chlorfenapyr, chlorfluazuron, chlorpyrifos, chlorpyrifos-methyl, chromafenozide, clothianidin, cyflumetofen, cyfluthrin, beta-cyfluthrin, cyhalothrin, lambda-cyhalothrin, cypermethrin, cyromazine, deltamethrin, diafenthiuron, diazinon, dieldrin, diflubenzuron, dimefluthrin, dimethoate, dinotefuran, diofenolan, emamectin, endosulfan, esfenvalerate, ethiprole, fenothiocarb, fenoxycarb, fenpropathrin, fenvalerate, fipronil, flonicamid, flubendiamide, flucythrinate, tau-fluvalinate, flufenerim (UR-50701), flufenoxuron, fonophos, halofenozide, hexaflumuron, hydramethylnon, imidacloprid, indoxacarb, isofenphos, lufenuron, malathion, metaflumizone, metaldehyde, methamidophos, methidathion, methomyl, methoprene, methoxychlor, metofluthrin, monocrotophos, methoxyfenozide, nitenpyram, nithiazine, novaluron, noviflumuron (XDE-007), oxamyl, parathion, parathion-methyl, permethrin, phorate, phosalone, phosmet, phosphamidon, pirimicarb, profenofos, profluthrin, pymetrozine, pyrafluprole, pyrethrin, pyridalyl, pyriprole, pyriproxyfen, rotenone, ryanodine, spinosad, spirodiclofen, spiromesifen (BSN 2060), spirotetramat, sulprofos, tebufenozide, teflubenzuron, tefluthrin, terbufos, tetrachlorvinphos, thiacloprid, thiamethoxam, thiodicarb, thiosultap-sodium, tralomethrin, triazamate, trichlorfon and triflumuron; fungicides such as acibenzolar, aldimorph, amisulbrom, azaconazole, azoxystrobin, benalaxyl, benomyl, benthiavalicarb, benthiavalicarb-isopropyl, binomial, biphenyl, bitertanol, blasticidin-S, Bordeaux mixture (Tribasic copper sulfate), boscalid/nicobifen, bromuconazole, bupirimate, buthiobate, carboxin, carpropamid, captafol, captan, carbendazim, chloroneb, chlorothalonil, chlozolinate, clotrimazole, copper oxychloride, copper salts such as copper sulfate and copper hydroxide, cyazofamid, cyflunamid, cymoxanil, cyproconazole, cyprodinil, dichlofluanid, diclocymet, diclomezine, dicloran, diethofencarb, difenoconazole, dimethomorph, dimoxystrobin, diniconazole, diniconazole-M, dinocap, discostrobin, dithianon, dodemorph, dodine, econazole, etaconazole, edifenphos, epoxiconazole, ethaboxam, ethirimol, ethridiazole, famoxadone, fenamidone, fenarimol, fenbuconazole, fencaramid, fenfuram, fenhexamide, fenoxanil, fenpiclonil, fenpropidin, fenpropimorph, fentin acetate, fentin hydroxide, ferbam, ferfurazoate, ferimzone, fluazinam, fludioxonil, flumetover, fluopicolide, fluoxastrobin, fluquinconazole, fluquinconazole, flusilazole, flusulfamide, flutolanil, flutriafol, folpet, fosetyl-aluminum, fuberidazole, furalaxyl, furametapyr, hexaconazole, hymexazole, guazatine, imazalil, imibenconazole, iminoctadine, iodicarb, ipconazole, iprobenfos, iprodione, iprovalicarb, isoconazole, isoprothiolane, kasugamycin, kresoxim-methyl, mancozeb, mandipropamid, maneb, mapanipyrin, mefenoxam, mepronil, metalaxyl, metconazole, methasulfocarb, metiram, metominostrobin/fenominostrobin, mepanipyrim, metrafenone, miconazole, myclobutanil, neo-asozin (ferric methanearsonate), nuarimol, octhilinone, ofurace, orysastrobin, oxadixyl, oxolinic acid, oxpoconazole, oxycarboxin, paclobutrazol, penconazole, pencycuron, penthiopyrad, perfurazoate, phosphonic acid, phthalide, picobenzamid, picoxystrobin, polyoxin, probenazole, prochloraz, procymidone, propamocarb, propamocarb-hydrochloride, propiconazole, propineb, proquinazid, prothioconazole, pyraclostrobin, pryazophos, pyrifenox, pyrimethanil, pyrifenox, pyrolnitrine, pyroquilon, quinconazole, quinoxyfen, quintozene, silthiofam, simeconazole, spiroxamine, streptomycin, sulfur, tebuconazole, techrazene, tecloftalam, tecnazene, tetraconazole, thiabendazole, thifluzamide, thiophanate, thiophanate-methyl, thiram, tiadinil, tolclofos-methyl, tolyfluanid, triadimefon, triadimenol, triarimol, triazoxide, tridemorph, trimoprhamide tricyclazole, trifloxystrobin, triforine, triticonazole, uniconazole, validamycin, vinclozolin, zineb, ziram, and zoxamide; nematocides such as aldicarb, oxamyl and fenamiphos; bactericides such as streptomycin; acaricides such as amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben and tebufenpyrad; and biological agents including entomopathogenic bacteria, such as Bacillus thuringiensis subsp. Aizawai, Bacillus thuringiensis subsp. Kurstaki, and the encapsulated delta-endotoxins of Bacillus thuringiensis (e.g., Cellcap, MPV, MPVII); entomopathogenic fungi, such as green muscardine fungus; and entomopathogenic virus including baculovirus, nucleopolyhedro virus (NPV) such as HzNPV, AfNPV; and granulosis virus (GV) such as CpGV.

The methods of controlling weeds can further include the application of a biologically effective amount of a herbicide of interest or a mixture of herbicides, and an effective amount of at least one additional biologically active compound or agent and can further comprise at least one of a surfactant, a solid diluent or a liquid diluent. Examples of such biologically active compounds or agents are: insecticides such as abamectin, acephate, acetamiprid, amidoflumet (S-1955), avermectin, azadirachtin, azinphos-methyl, bifenthrin, binfenazate, buprofezin, carbofuran, chlorfenapyr, chlorfluazuron, chlorpyrifos, chlorpyrifos-methyl, chromafenozide, clothianidin, cyfluthrin, beta-cyfluthrin, cyhalothrin, lambda-cyhalothrin, cypermethrin, cyromazine, deltamethrin, diafenthiuron, diazinon, diflubenzuron, dimethoate, diofenolan, emamectin, endosulfan, esfenvalerate, ethiprole, fenothicarb, fenoxycarb, fenpropathrin, fenvalerate, fipronil, flonicamid, flucythrinate, tau-fluvalinate, flufenerim (UR-50701), flufenoxuron, fonophos, halofenozide, hexaflumuron, imidacloprid, indoxacarb, isofenphos, lufenuron, malathion, metaldehyde, methamidophos, methidathion, methomyl, methoprene, methoxychlor, monocrotophos, methoxyfenozide, nithiazin, novaluron, noviflumuron (XDE-007), oxamyl, parathion, parathion-methyl, permethrin, phorate, phosalone, phosmet, phosphamidon, pirimicarb, profenofos, pymetrozine, pyridalyl, pyriproxyfen, rotenone, spinosad, spiromesifin (BSN 2060), sulprofos, tebufenozide, teflubenzuron, tefluthrin, terbufos, tetrachlorvinphos, thiacloprid, thiamethoxam, thiodicarb, thiosultap-sodium, tralomethrin, trichlorfon and triflumuron; fungicides such as acibenzolar, azoxystrobin, benomyl, blasticidin-S, Bordeaux mixture (tribasic copper sulfate), bromuconazole, carpropamid, captafol, captan, carbendazim, chloroneb, chlorothalonil, copper oxychloride, copper salts, cyflufenamid, cymoxanil, cyproconazole, cyprodinil, (S)-3,5-dichloro-N-(3-chloro-1-ethyl-1-methyl-2-oxopropyl)-4-methylbenzamide (RH 7281), diclocymet (S-2900), diclomezine, dicloran, difenoconazole, (S)-3,5-dihydro-5-methyl-2-(methylthio)-5-phenyl-3-(phenyl-amino)-4H-imidazol-4-one (RP 407213), dimethomorph, dimoxystrobin, diniconazole, diniconazole-M, dodine, edifenphos, epoxiconazole, famoxadone, fenamidone, fenarimol, fenbuconazole, fencaramid (SZX0722), fenpiclonil, fenpropidin, fenpropimorph, fentin acetate, fentin hydroxide, fluazinam, fludioxonil, flumetover (RPA 403397), flumorf/flumorlin (SYP-L190), fluoxastrobin (HEC 5725), fluquinconazole, flusilazole, flutolanil, flutriafol, folpet, fosetyl-aluminum, furalaxyl, furametapyr (S-82658), hexaconazole, ipconazole, iprobenfos, iprodione, isoprothiolane, kasugamycin, kresoxim-methyl, mancozeb, maneb, mefenoxam, mepronil, metalaxyl, metconazole, metomino-strobin/fenominostrobin (SSF-126), metrafenone (AC375839), myclobutanil, neo-asozin (ferric methane-arsonate), nicobifen (BAS 510), orysastrobin, oxadixyl, penconazole, pencycuron, probenazole, prochloraz, propamocarb, propiconazole, proquinazid (DPX-KQ926), prothioconazole (JAU 6476), pyrifenox, pyraclostrobin, pyrimethanil, pyroquilon, quinoxyfen, spiroxamine, sulfur, tebuconazole, tetraconazole, thiabendazole, thifluzamide, thiophanate-methyl, thiram, tiadinil, triadimefon, triadimenol, tricyclazole, trifloxystrobin, triticonazole, validamycin and vinclozolin; nematocides such as aldicarb, oxamyl and fenamiphos; bactericides such as streptomycin; acaricides such as amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben and tebufenpyrad; and biological agents including entomopathogenic bacteria, such as Bacillus thuringiensis subsp. Aizawai, Bacillus thuringiensis subsp. Kurstaki, and the encapsulated delta-endotoxins of Bacillus thuringiensis (e.g., Cellcap, MPV, MPVII); entomopathogenic fungi, such as green muscardine fungus; and entomopathogenic virus including baculovirus, nucleopolyhedro virus (NPV) such as HzNPV, AfNPV; and granulosis virus (GV) such as CpGV. Methods of the invention may also comprise the use of plants genetically transformed to express proteins (such as Bacillus thuringiensis delta-endotoxins) toxic to invertebrate pests. In such embodiments, the effect of exogenously applied invertebrate pest control compounds may be synergistic with the expressed toxin proteins. General references for these agricultural protectants include The Pesticide Manual, 13th Edition, C. D. S. Tomlin, Ed., British Crop Protection Council, Farnham, Surrey, U.K., 2003 and The BioPesticide Manual, 2^{nd} Edition, L. G. Copping, Ed., British Crop Protection Council, Farnham, Surrey, U.K., 2001. In certain instances, combinations with other invertebrate pest control compounds or agents having a similar spectrum of control but a different mode of action will be particularly advantageous for resistance management. Thus, compositions of the present invention can further comprise a biologically effective amount of at least one additional invertebrate pest control compound or agent having a similar spectrum of control but a different mode of action. Contacting a plant genetically modified to express a plant protection compound (e.g., protein) or the locus of the plant with a biologically effective amount of a compound of this invention can also provide a broader spectrum of plant protection and be advantageous for resistance management.

Thus, methods of controlling weeds can employ a herbicide or herbicide combination and may further comprise the use of insecticides and/or fungicides, and/or other agricultural chemicals such as fertilizers. The use of such combined treatments of the invention can broaden the spectrum of activity against additional weed species and suppress the proliferation of any resistant biotypes.

Methods can further comprise the use of plant growth regulators such as aviglycine, N-(phenylmethyl)-1H-purin-6-amine, ethephon, epocholeone, gibberellic acid, gibberellin A₄ and A₇, harpin protein, mepiquat chloride, prohexadione calcium, prohydrojasmon, sodium nitrophenolate and trinexapac-methyl, and plant growth modifying organisms such as Bacillus cereus strain BP01.

### IIX. Sequence Comparisons

The following terms are used to describe the sequence relationships between two or more polynucleotides or polypeptides: (a) “reference sequence”, (b) “comparison window”, (c) “sequence identity”, and, (d) “percent sequence identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence or protein sequence.

(b) As used herein, “comparison window” makes reference to a contiguous and specified segment of a polypeptide sequence, wherein the polypeptide sequence in the comparison window may comprise additions or deletions (i.e., gaps) compared to the reference sequence (which does not comprise additions or deletions) for optimal alignment of the two polypeptides. Generally, the comparison window is at least 5, 10, 15, or 20 contiguous amino acid in length, or it can be 30, 40, 50, 100, or longer. Those of skill in the art understand that to avoid a high similarity to a reference sequence due to inclusion of gaps in the polypeptide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16: 10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et at (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. BLASTP protein searches can be performed using default parameters. See, blast.ncbi.nlm.nih.gov/Blast.cgi.

To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, or PSI-BLAST, the default parameters of the respective programs (e.g., BLASTN for nucleotide sequences, BLASTP for proteins) can be used. See www.ncbi.nlm.nih.gov. Alignment may also be performed manually by inspection.

Unless otherwise stated, sequence identity values provided herein refer to the value obtained using GAP Version 10 using the following parameters: % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch (1970) J. Mol. Biol. 48:443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package is BLOSUM62 (see Henikoff and Henikoff (1989) Proc. Natl. Acad. Sci. USA 89:10915).

(c) As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity). When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percent sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percent sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percent sequence identity.

(e) Two sequences are “optimally aligned” when they are aligned for similarity scoring using a defined amino acid substitution matrix (e.g., BLOSUM62), gap existence penalty and gap extension penalty so as to arrive at the highest score possible for that pair of sequences. Amino acids substitution matrices and their use in quantifying the similarity between two sequences are well-known in the art and described, e.g., in Dayhoff et al. (1978) “A model of evolutionary change in proteins.” In “Atlas of Protein Sequence and Structure,” Vol. 5, Suppl. 3 (ed. M. O. Dayhoff), pp. 345-352. Natl. Biomed. Res. Found., Washington, D.C. and Henikoff et al. (1992) Proc. Natl. Acad. Sci. USA 89:10915-10919. The BLOSUM62 matrix is often used as a default scoring substitution matrix in sequence alignment protocols such as Gapped BLAST 2.0. The gap existence penalty is imposed for the introduction of a single amino acid gap in one of the aligned sequences, and the gap extension penalty is imposed for each additional empty amino acid position inserted into an already opened gap. The gap existence penalty is imposed for the introduction of a single amino acid gap in one of the aligned sequences, and the gap extension penalty is imposed for each additional empty amino acid position inserted into an already opened gap. The alignment is defined by the amino acids positions of each sequence at which the alignment begins and ends, and optionally by the insertion of a gap or multiple gaps in one or both sequences, so as to arrive at the highest possible score. While optimal alignment and scoring can be accomplished manually, the process is facilitated by the use of a computer-implemented alignment algorithm, e.g., gapped BLAST 2.0, described in Altschul et al, (1997) Nucleic Acids Res. 25:3389-3402, and made available to the public at the National Center for Biotechnology Information Website (http://www.ncbi.nlm.nih.gov). Optimal alignments, including multiple alignments, can be prepared using, e.g., PSI-BLAST, available through http://www.ncbi.nlm.nih.gov and described by Altschul et al, (1997) Nucleic Acids Res. 25:3389-3402.

Non-limiting embodiments include:

1. A method to detoxify an auxin-analog herbicide comprising applying to a plant, a plant cell or a seed an auxin-analog herbicide, wherein said plant, plant cell or seed comprises a heterologous polynucleotide encoding a GH3 polypeptide having amino acid/auxin-analog conjugation activity, and wherein expression of the GH3 polypeptide produces an aspartate/auxin-analog conjugate or a glutamate/auxin-analog conjugate wherein said auxin-analog conjugate has reduced herbicidal activity.

2. A method for controlling at least one weed in an area of cultivation comprising a crop or a seed of the crop, said method comprising applying to the area of cultivation and/or applying to the crop or a seed of the crop in the area of cultivation a sufficient amount of an auxin-analog herbicide to control weeds without significantly affecting the crop, wherein said crop or seed thereof in said area of cultivation comprises at least one heterologous polynucleotide encoding a GH3 polypeptide having amino acid/auxin-analog conjugation activity.

3. A method for controlling at least one weed in an area of cultivation containing a crop comprising

(a) applying to the area of cultivation a sufficient amount of an auxin-analog herbicide to control weeds without significantly affecting the crop;

(b) planting the field with a crop or a seed thereof having a heterologous polynucleotide encoding a GH3 polypeptide having amino acid/auxin-analog conjugation activity.

4. The method of any one of embodiments 2 or 3, wherein said crop exhibits an increased insensitivity to the auxin-analog herbicide as compared to an appropriate control plant.

5. The method of embodiment 3, wherein step (a) occurs before or simultaneously with step (b).

6. The method of embodiment 1, 2, 3, 4, or 5, wherein said GH3 polypeptide comprises a polypeptide having an amino acid sequence having at least 85%, 90%, 95% or 100% sequence identity to any one of SEQ ID NO: 1, 2, 3, 4, 5, 6, 7, 8, 9, 10, 11, 12, 13, 14, 33, 34, 35, 36, 37, 38, 39, 40, 41, 42, 43, 44, 45, 46, 47, 48, 49, 50, 58, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 117, 118, 119, 120, 121, 124, 142, 144, or 145.

7. The method of embodiment 1, 2, 3, 4, or 5, wherein said GH3 polypeptide comprises a polypeptide having an amino acid sequence having at least 80%, 85%, 90%, 95% or 100% sequence identity to any one of SEQ ID NO: 15, 16, 17, 51, 52, 53, 54, 55, 56, 57, 59, 60, 61, 62, 63, 64, 65, 66, 67, 98, 99, 100, 101, 102, 103, 104, 105, 106, 107, 108, 109, 110, 111, 112, 113, 114, 115, 116, 122, 134, 135, 136, 137, 138, 139, 140, 141.

8. The method of embodiment 1, 2, 3, 4, or 5, wherein said GH3 polypeptide comprises a polypeptide having an amino acid sequence having at least 80%, 85%, 90%, 95% or 100% sequence identity to any one of SEQ ID NO: 18, 19, 20, 21, 22, 23, 24, 25, 26, 27, 28, 29, 30, 31, 32, 123, 125, 126, 127, 128, 129, 130, 131, 132, 133, or 143.

9. The method of any one of embodiments 1-8, wherein said auxin-analog herbicide comprises 2,4-D or dicamba.

10. The method of any one of embodiments 2-5, wherein said crop further comprises at least one polypeptide imparting tolerance to an additional herbicide. 11. The method of embodiment 10, wherein said at least one polypeptide imparting tolerance to an additional herbicide comprises:
(a) a sulfonylurea-tolerant acetolactate synthase;
(b) an imidazolinone-tolerant acetolactate synthase;
(c) a glyphosate-tolerant 5-enolpyruvylshikimate-3-phosphate synthase;
(d) a glyphosate-tolerant glyphosate oxido-reductase;
(e) a glyphosate-N-acetyltransferase;
(f) a phosphinothricin acetyl transferase;
(g) a protoporphyrinogen oxidase;
(h) an HPPD enzyme;
(i) a P450 polypeptide; or,
(j) an acetyl coenzyme A carboxylase (ACCase).

12. The method of embodiment 11, wherein said at least one polypeptide imparting tolerance to an additional herbicide comprises a high resistance allele of acetolactate synthase (HRA) and/or a glyphosate-N-acetyltransferase polypeptide.

13. The method of any one of embodiments 2-5, wherein the crop further comprises at least one additional polypeptide imparting tolerance to an auxin-analog herbicide, wherein said additional polypeptide is either the same or different than said GH3 polypeptide encoded by the heterologous polynucleotide.

14. The method of embodiment 1, wherein said plant, plant cell or seed is from a monocot.

15. The method of embodiment 1, wherein said plant, plant cell or seed is from a dicot.

16. The method of embodiment 2 or 3, wherein said crop is from a monocot.

17. The method of embodiment 2 or 3, wherein said crop is from a dicot.

18. The method of any one of embodiments 14 or 16, wherein said monocot plant is selected from the group consisting of maize, wheat, rice, barley, sorghum, or rye.

19. The method of any one of embodiments 15 or 17, wherein said dicot plant is selected from the group consisting of soybean, Brassica, sunflower, cotton, or alfalfa.

20. The method of any one of embodiments 1-19, wherein said auxin-analog herbicide comprises 2,4-D.

21. The method of any one of embodiments 1-19, wherein said auxin-analog herbicide comprises dicamba.

22. A method for testing a plant response to one or more compounds, comprising,
a) providing a composition comprising one or more compounds to be tested in a container having at least an opening for the compound enter and/or exit, and one or more elements for moving the composition into or out of the container;
b) contacting at least one opening of the container with the surface of a plant and moving the composition comprising one or more compounds to at least the surface of the plant.

23. The method of embodiment 22, wherein the container and elements for moving the composition comprise a syringe body and plunger.

24. The method of embodiment 22, wherein moving the composition to the plant surface comprises contacting the surface with pressure sufficient for the composition to enter the cells of the plant.

25. The method of embodiment 22, further comprising measuring an effect of the composition on the contacted plant compared to a plant that is not contacted with the composition.

### EXPERIMENTAL

### Abbreviations

The following abbreviations are used: IAA: indole-3-acetic acid; 2,4-D: 2,4-dichlorophenoxyacetic acid; and dicamba: 3,6-dichloro-2-methoxybenzoic acid free acid. Conjugated forms of IAA, 2,4-D, and dicamba are indicated by appending as a suffix a dash with the three letter code for amino acid conjugated to the compound. For example, IAA conjugated to aspartic acid is indicated as IAA-Asp or 2,4-D conjugated to glutamic acid is indicated as 2,4-D-Glu.

### Example 1

### Methods for Measuring Auxin Conjugation Activities

Auxin amino acid conjugation is a two step enzymatic reaction. The first step involves adenylation: the transfer of AMP from ATP to the carboxylic acid group of an acyl substrate, forming an activated acyl-adenylate intermediate and releasing pyrophosphate (PPᵢ). The second step involves a transferase reaction replacing AMP of the intermediate with an amino acid by the formation of an amide bond (See FIGS. 1 and 2).

Adenylation activity was monitored spectrophotometrically at 340 nm by coupling the production of pyrophosphate to oxidation of NADH using a pyrophosphate reagent (Sigma reagent catalog number P7275). Coupling enzymes including pyrophosphate dependent fructose-6-phosphate kinase, aldolase, triosephosphate isomerase, glycerophosphate dehydrogenase, and appropriate substrates and cofactors including NADH are all included in the reagent kit. To start the assay, the pyrophosphate reagent was reconstituted in 4 mL of double-distilled H₂O (“ddH₂O”). A master mix containing 6.45 mM MgCl₂, 3.23 mM ATP, 1.29 mM DTT, and 65 μL pyrophosphate reagent in a total of 155 μL was prepared and added into a UV plate containing 10 μg GH3 enzyme and 20 μl of 10 mM auxin or auxin herbicide for final concentration of 1 mM. The reaction volume was brought to 200 μl by the addition of ddH₂O. The reaction plate was measured using a SpectraMax Plus 384 device (Molecular Devices) for changes in absorbance at 340 nm every 30 s for 1 hr at 30° C. Measured absorbance was then converted to velocity by least squares fitting of each curve using the accompanying program SOFTmax PRO 5.4 with manual assessment/confirmation of the linear range. The velocity of a no-auxin control was subtracted. An extinction coefficient of 6.22 mM⁻¹ cm⁻¹ for NADH was used to convert velocity values from milli-absorbance units/min to micromolar/min. Kinetic parameters were estimated by fitting initial velocity values to the Michaelis-Menten equation.

The second step of the conjugation was measured spectrophotometrically at 340 nm for oxidation of NADH by enzyme coupled assay (Chen et al. 2010. J Biol Chem 285:29780-29786). This method measures the release of AMP from the acyl-adenylate intermediate. Coupled enzymes include: myokinase, pyruvate kinase, and lactic acid dehydrogenase. To start the assay, a master mix containing 20 mM Tris-HCl (pH 8.0), 3 mM MgCl₂, 2 mM ATP, 4 mM of auxin or auxin herbicide substrate, 6 mM amino acid, 2 mM DTT, 2 mM phosphoenolpyruvate, 400 μM NADH, 8 units/100 μL of rabbit muscle myokinase, 8 units/100 μL of rabbit muscle pyruvate kinase, and 8 units/100 μL of rabbit muscle lactate dehydrogenase was prepared. 100 μl master mix was added to a UV microplate containing 10 μg GH3 protein in 100 μl reaction buffer (20 mM Tris-HCl, 3 mM MgCl₂ pH 8.0). The reaction plate was measured in SpectraMax Plus 384 device (Molecular Devices) for changes in absorbance at 340 nm every 30 s for 1 hr at 30° C. Measured absorbance was then converted to velocity by least squares fitting of each curve using the accompanying program SOFTmax PRO 5.4 with manual assessment/confirmation of the linear range. The velocity of a no-auxin control was subtracted. An extinction coefficient of 6.22 mM⁻¹ cm⁻¹ for NADH was used to convert velocity values from milli-absorbance units/min to micromolar/min. Kinetic parameters were estimated by fitting initial velocity values to the Michaelis-Menten equation.

To detect and monitor the formation of acyl conjugates with 2,4-D or dicamba, a sensitive LC-MS method was developed using the respective amino acid conjugate standards. With this procedure, the level of 2,4-D-Asp, 2,4-D-Glu, dicamba-Asp, and dicamba-Glu were detected as low as 2.5 fmol, 5 fmol, 20 fmol, and 20 fmol, respectively. The detection limit for IAA-Asp and IAA-Glu is comparable at 25 fmol. The reaction was performed at 30° C., 105 rpm, for 3 hours in buffer containing 20 mM Tris-HCl (pH 8.0), 5 mM MgCl₂, 5 mM auxin (IAA, 2,4-D or dicamba), 5 mM ATP, 5 mM Asp or Glu, 1 mM DTT and 44 to 164 μg/mL purified GH3 protein in 100 μL reaction. After 3 hours of reaction, 400 μL 100% MeOH was added to the reaction mixture for protein precipitation. The supernatant was diluted 20× in water and then analyzed by LC-MS (AB Sciex 4000 QTrap coupled with Shimadzu Nexera UHPLC) using a Phenomenex Luna 3 μm Phenyl-Hexyl (00B-4256-Y0) column. Details of the LC-MS method are listed in table 2. The concentration of the conjugates was calculated based on the standards. Kinetic parameters were estimated by fitting initial velocity values to the Michaelis-Menten equation.

TABLE 2
LC-MS methods for IAA, 2,4-D, dicamba and their conjugates.

| | | |
|---|---|---|
| Time (mins) | Solvent B % | MRM transition (m/z) |

Method for IAA, IAA-Asp and IAA-Glu detection

| | | | |
|---|---|---|---|
| 1 | 2 | IAA | 174 to 130 |
| 3 | 70 | IAA-Asp | 289 to 132 |
| 3.9 | 70 | IAA-Glu | 303 to 146 |
| 4 | 2 | | |
| 5 | stop | | |

Method for 2,4-D, 2,4-D-Asp and 2,4-D-Glu detection

| | | | |
|---|---|---|---|
| 1 | 5 | 2,4-D | 218 to 161 |
| 3 | 100 | 2,4-D-Asp | 334 to 161 |
| 3.9 | 100 | 2,4-D-Glu | 348 to 161 |
| 4 | 5 | | |
| 5 | stop | | |

Method for Dicamba, Dicamba-Asp and Dicamba-Glu detection

| | | | |
|---|---|---|---|
| 1 | 2 | Dicamba | 218 to 175 |
| 3 | 70 | Dicamba-Asp | 334 to 114 |
| 3.9 | 70 | Dicamba-Glu | 348 to 330 |
| 4 | 2 | | |
| 5 | stop | | |

Solvent A: 5 mM NH₄Ac in H₂O
Solvent B: 5 mM NH₄Ac in MeOH

### Example 2

### Phytotoxicity Evaluation of 2,4-D and Dicamba Conjugated with Aspartic Acid and Glutamic Acid

To evaluate the effect of synthetic auxin conjugates in plants, 2,4-D-Asp, 2,4-D-Glu, dicamba-Asp, and dicamba-Glu were synthesized (Irvine Chemistry Laboratory, Anaheim, Calif.) and tested for auxin effects during soybean germination.

Asp and Glu conjugates of 2,4-D and dicamba were dissolved in a small quantity of Na₂CO₃ and ethanol, respectively, diluted in ddH₂O to obtain 10 mM stock solution, and filter sterilized. Soybean seeds of a Pioneer elite germplasm were sterilized with chlorine gas as following: a) two layers of seeds were placed in a 100×25 mm plastic Petri dish; b) in an exhaust fume hood, seeds were placed into a glass desiccator with a 250 mL beaker containing 100 mL bleach (5% NaOC1) and 3.5 mL 12N HCl was slowly added to the beaker; c) the lid was sealed closed on the desiccator and the seeds sterilized for at least 24 hr.

Sterilized soybean seeds were then imbibed in ddH₂O under sterile conditions at 25° C. for 24 hours before germination test. For germination test, 6-8 imbibed seeds were placed on a 100×25 mm deep Petri dish plate containing 50 ml germination media supplemented with or without auxin herbicide conjugates. 1 L seed germination media contains 3.21 g GAMBORG B-5 basal medium (PhytoTech), 20 g sucrose, 5 g tissue culture agar, and is pH adjusted to 5.7. Media was autoclaved at 121° C. for 25 min and cooled to 60° C. before the addition of auxin conjugates. Germination was carried out in a Percival growth chamber at 25° C. under 18 hr light and 6 hr dark cycle at 90 to 150 μE/m2/s and grown for 10 days.

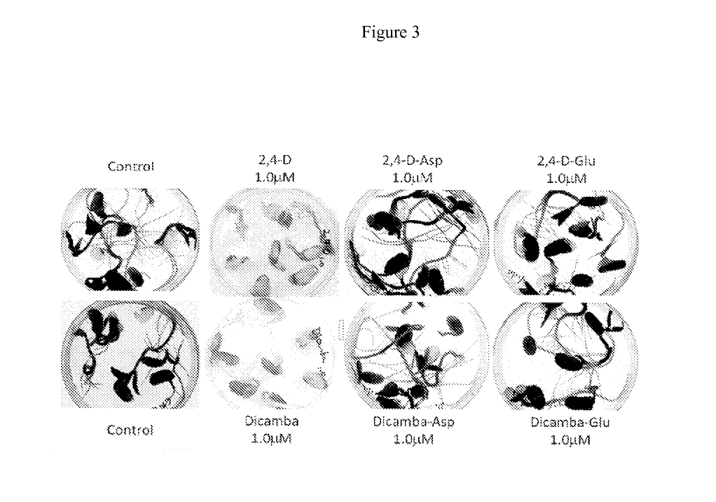

As shown in FIG. 3, soybean seeds germinated and grew very well in the media containing no supplemented auxin herbicides or auxin herbicide conjugates (control in FIG. 3). Cotyledons were fully expanded, the first true leaf emerged, and the roots grew well into the media. In plates where 1 μM 2,4-D or dicamba was added, seed germination was arrested as evident by bleaching of cotyledon and malformed and growth arrested roots. Emergence of true leaf and formation of secondary roots were not observed from these seeds. In comparison, in plates containing 1 μM of auxin herbicide conjugates 2,4-D-Asp, 2,4-D-Glu, dicamba-Asp, and dicamba-Glu, seed germination and growth were normal, similar to that of the control plates. The results indicate that these auxin herbicide conjugates are not phytotoxic to soybean. Converting 2,4-D or dicamba into Asp or Glu conjugates can effectively turn the herbicides into non-herbicidal compounds in plants.

### Example 3

### Phylogenetic Analysis of GH3 Family Proteins

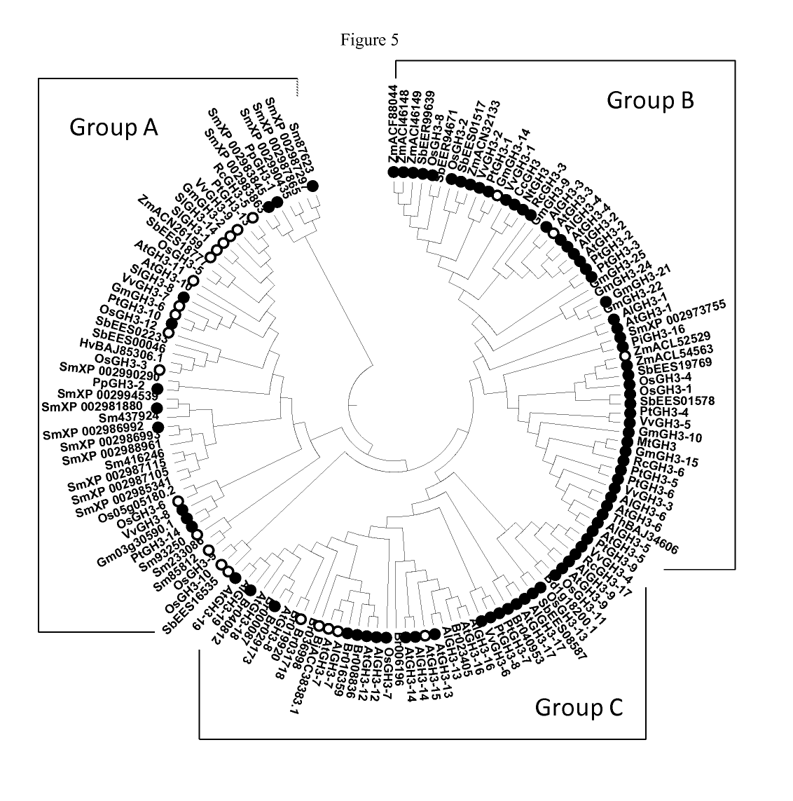

A total of 246 GH3 protein sequences were obtained by BLAST analysis (Jun. 20, 2012) using AtGH3.17 protein sequence (Accession # Q9FZ87) to the NCBI database (http://www.ncbi.nlm.nih.gov/) and Pioneer internal database, and analyzed for their phylogenetic relationship using CLUSTAL W (FIG. 4). From this phylogenetic analysis, 145 GH3 homologs were selected for activity evaluation (FIG. 5, Table 3 and Table 6).

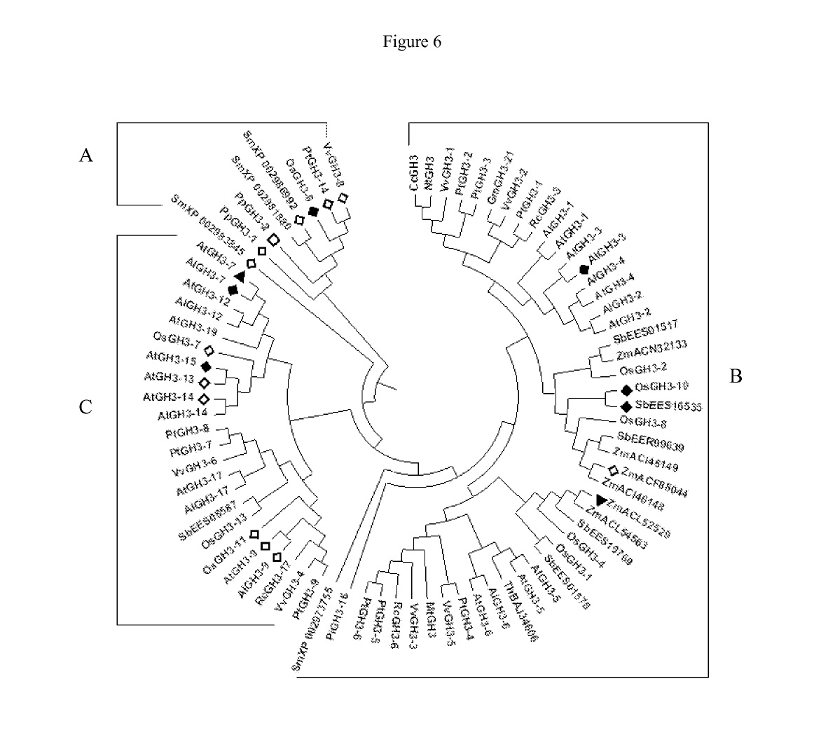

Based on previous studies of the substrate specificity of GH3 family proteins, three major subgroups have been proposed (Staswick et al. (2002) Plant Cell 14: 1405-1415; Wang et al. (2008) Plant Growth Regul 56:225-232). Subgroup I catalyzes the ligation of amino acids to jasmonic acid (Staswick et al. (2002) Plant Cell 14: 1405-1415). Auxins such as IAA, PAA, IBA and salicylic acid are the substrates of subgroup II (Staswick et al. (2002) Plant Cell 14: 1405-1415; Staswick et al. (2005) Plant Cell 17:616-627). Subgroup III protein AtGH3-12 can conjugate 4-hydroxybenzoate, and other benzoates (Okrent et al. (2009) J Biol Chem 284:9742-9754). As shown in FIG. 4, GH3 homologs fall into three major groups (Group A, B, C) in the phylogenetic analysis. From previous studies (Staswick et al. (2002) Plant Cell 14: 1405-1415; Staswick et al. (2005) Plant Cell 17:616-62; Wang et al. (2008) Plant Growth Regul 56:225-232; Okrent et al. (2009) J Biol Chem 284:9742-9754), proteins in phylogenetic group A, B, and C are predicted to belong to substrate subgroup I, II, and III, respectively, with substrate preference of jasmonic acid, IAA, and benzoates, respectively. This is not always true. For example, in the group of 78 tested GH3 proteins, 18 did not conjugate IAA with Asp or Glu. Most (14 out of 18) of these IAA-inactive GH3 proteins phylogenetically belong to subgroup A and C, confirming the general alignment of sequence group and substrate specificity (FIG. 6). However, the phylogenetic grouping does not align completely with the subgroups based on substrate specificity, as exemplified by the other four IAA-inactive proteins AtGH3-3, OsGH3-10, SbEES16535, and ZmACF88044, all belonging to subgroupB. Another example is with AtGH3.17, phylogenetically a subgroup C protein (FIGS. 4 and 6). However, AtGH3-17 conjugates IAA with Asp or Glu very well, with k_{cat} values at 52 and 868 hr⁻¹ respectively (Table 3 and 4; Staswick et al. (2005) Plant Cell 17:616-627), and thus is a subgroup II protein. Even though the pair-wise sequence identity among GH3 family proteins varies from 30% to 98%, we have identified proteins that can conjugate IAA to Asp or Glu from all substrate specificity subgroups. For example, subgroup C proteins AtGH3-7, AtGH3.12, AtGH3-19, AlGH3-14 and AlGH3-12 can also conjugate Glu to IAA (FIG. 6), the substrate of subgroup II but not subgroup III. Phylogenetic group A proteins PpGH3-2 and SmXP_{—}002981880 can also conjugate IAA to Asp and Glu (FIG. 6). Furthermore, proteins from sequence subgroups A, B and C all showed 2,4-D conjugation activity (FIG. 5 and Table 4 and 5). Thus, we conclude that phylogenetic placement itself is not enough to predict the substrate specificity of GH3 proteins and to identify proteins that can conjugate 2,4-D or dicamba to Asp or Glu.

### Example 4

### Examination of 145 GH3 Superfamily Protein Members with IAA, 2,4-D, and Dicamba in Conjugation Reaction with Aspartic Acid and Glutamic Acid

The GH3 superfamily conjugase activity analysis of IAA, 2,4-D or dicamba with Asp or Glu was done using LC-MS to analyze the final conjugate products as described in Example 1. Protein sequences of GH3 homologs (Table 3) were obtained from public databases (NCBI, http://www.ncbi.nlm.nih.gov/) by Blast analysis of AtGH3.17 protein sequence (accession # Q9FZ87). Coding sequences were designed for optimal expression in E. coli based on the protein sequences and synthesized. Synthesized coding sequences along with N-term his-tag coding sequences were cloned into a pET24a-based E. coli expression vector. The E. coli expression vectors were transformed into OverExpress C41(DE3) cells (Lucigen) for protein expression. Recombinant E. coli strains were inoculated into 5 ml LB media supplemented with 40 mg/L kanamycin and cultured overnight at 37° C. The overnight culture (0.5 mL) was inoculated into 50 mL LB medium plus 40 mg/L kanamycin and grown at 30° C. until OD₆₀₀ reached 0.6. The cultures were induced with 0.2 mM IPTG at 16° C., 230 rpm overnight. Cells were harvested by centrifugation at 7,000 rpm for 10 mins. The cell pellet from 50 mL of cell culture was frozen and thawed twice and then lysed in 800 μL lysis buffer A (150 mM NaCl, 50 mM Tris-HCl pH 8.0, 5 mM MgCl₂, 10% glycerol) plus 20 mM imidazole, 1 mM DTT, 0.2 mg/ml lysozyme, 1/200 protease inhibitor cocktail (EMD set3, EDTA free) and 1/2000 endonuclease. Lysate was then centrifuged at 13,000 rpm for 30 min at 4° C. Supernatant was loaded onto 200 μL Ni-NTA columns pre-equilibrated with buffer A with 20 mM imidazole at 4° C. Each column was then washed with (1) 2×800 μL of buffer A with 10 mM imidazole, (2) 2×800 μL of buffer A with 20 mM imidazole and (3) 2×200 μL of buffer A with 50 mM imidazole. Protein was eluted with 200 μL of buffer A with 250 mM imidazole. The concentration was measured by Bradford assay. Purified protein was used in conjugation reaction assays as described in example 1.

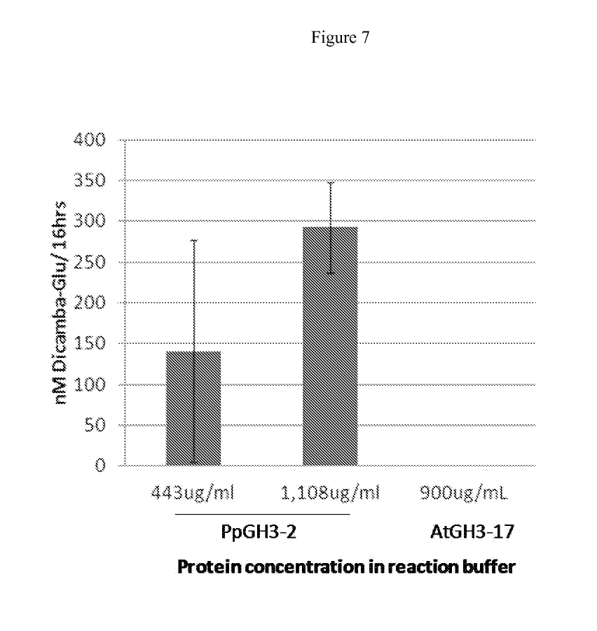

As shown in Tables 4 and 5, among the 78 tested GH3 proteins, most of them (70 GH3 proteins) showed various level of 2,4-D conjugation activity with Asp or Glu. Sixteen of them produced more than 1 μM of 2,4-D-Asp or 2,4-D-Glu from a reaction having 5 mM 2,4-D substrate (Table 4). The activity is usually less than the corresponding activity for IAA conjugation to Asp or Glu. In some cases, the conjugase is over 1,000-fold higher in k_{cat} with IAA as compared to 2,4-D (Table 5). However, twelve GH3 proteins that did not show any activity of conjugating IAA with Asp and Glu were found to produce 2,4-D-Asp or 2,4-D-Glu conjugates (FIG. 6, Table 4 and 5). These IAA-inactive 2,4-D conjugases are: AlGH3-9, AtGH3-9, OsGH3-7, OsGH3-11, PtGH3-14, VvGH3-8, AtGH3-13, AtGH3-14, ZmACF88044, PpGH3-1, SmXP_{—}002983845, and SmXP_{—}002986992 (SEQ ID NO 1, 4, 5, 6, 15, 16, 25, 26, 45, 51, 57, and 66). One active GH3 conjugase for both IAA and 2,4-D, PpGH3-2 (SEQ ID NO:52), could conjugate dicamba with Glu in the 16 hour reaction. Increased amount of dicamba-Glu was detected when increased amount of PpGH3-2 protein was used in the conjugation reaction (FIG. 7).

TABLE 3
Protein names of 145 selected GH3 proteins for activity test.

| SEQ ID | Protein name | Gene Bank Accession Number |
|---|---|---|
| 1 | AlGH3-9 | EFH58389 |
| 2 | AlGH3-17 | XP_002890761 |
| 3 | AtGH3-17 | Q9FZ87 |
| 4 | AtGH3-9 | O82243 |
| 5 | OsGH3-7 | Q654M1 |
| 6 | OsGH3-11 | P0C0M3 |
| 7 | OsGH3-13 | EAY81150 |
| 8 | PtGH3-7 | EEE84166 |
| 9 | PtGH3-8 | EEE78719 |
| 10 | PtGH3-9 | EEE81977 |
| 11 | RcGH3-17 | XP_002531876 |
| 12 | SbEES08587 | EES08587 |
| 13 | VvGH3-4 | XP_002263353 |
| 14 | VvGH3-6 | XP_002268278 |
| 15 | PtGH3-14 | EEE99364 |
| 16 | VvGH3-8 | XP_002271002 |
| 17 | OsGH3-6 | Q60EY1 |
| 18 | AlGH3-13 | EFH47822 |
| 19 | AlGH3-14 | EFH47823 |
| 20 | AlGH3-16 | EFH47825 |
| 21 | AlGH3-19 | EFH70384 |
| 22 | AlGH3-12 | XP_002871561 |
| 23 | AlGH3-7 | XP_002890567 |
| 24 | AtGH3-12 | Q9LYU4 |
| 25 | AtGH3-13 | CAB86642 |
| 26 | AtGH3-14 | CAB87143 |
| 27 | AtGH3-15 | CAB87144 |
| 28 | AtGH3-16 | CAB87145 |
| 29 | AtGH3-18 | AAG60120 |
| 30 | AtGH3-19 | AAG60122 |
| 31 | AtGH3-7 | AAC00604 |
| 32 | AtGH3-8 | BAB08663 |
| 33 | OsGH3-1 | Q8LQM5 |
| 34 | OsGH3-10 | Q6ZLA7 |
| 35 | OsGH3-2 | BAF06259 |
| 36 | OsGH3-4 | AAU90225 |
| 37 | OsGH3-8 | Q0D4Z6 |
| 38 | OsGH3-9 | Q6ZLA3 |
| 39 | SbEER94671 | EER94671 |
| 40 | SbEER99639 | EER99639 |
| 41 | SbEES01517 | EES01517 |
| 42 | SbEES01578 | EES01578 |
| 43 | SbEES16535 | EES16535 |
| 44 | SbEES19769 | EES19769 |
| 45 | ZmACF88044 | ACF88044 |
| 46 | ZmACI46148 | ACI46148 |
| 47 | ZmACI46149 | ACI46149 |
| 48 | ZmACL52529 | ACL52529 |
| 49 | ZmACL54563 | ACL54563 |
| 50 | ZmACN32133 | ACN32133 |
| 51 | PpGH3-l | CAD42870 |
| 52 | PpGH3-2 | CAD42871 |
| 53 | SmXP_002990435 | XP_002990435 |
| 54 | SmXP_002987865 | XP_002987865 |
| 55 | SmXP_002987297 | XP_002987297 |
| 56 | SmXP_002983863 | XP_002983863 |
| 57 | SmXP_002983845 | XP_002983845 |
| 58 | SmXP_002973755 | XP_002973755 |
| 59 | SmXP_002985341 | XP_002985341 |
| 60 | SmXP_002987105 | XP_002987105 |
| 61 | SmXP_002987115 | XP_002987115 |
| 62 | SmXP_002981880 | XP_002981880 |
| 63 | SmXP_002994539 | XP_002994539 |
| 64 | SmXP_002988961 | XP_002988961 |
| 65 | SmXP_002986993 | XP_002986993 |
| 66 | SmXP_002986992 | XP_002986992 |
| 67 | SmXP_002990290 | XP_002990290 |
| 68 | AlGH3-1 | XP_002885948 |
| 69 | AlGH3-3 | XP_002880486 |
| 70 | AlGH3-4 | XP_002886679 |
| 71 | AlGH3-5 | XP_002869571 |
| 72 | AlGH3-6 | XP_002866046 |
| 73 | AlGH3-2 | XP_002868993 |
| 74 | AtGH3-1 | O82333 |
| 75 | AtGH3-2 | AEE86787 |
| 76 | AtGH3-3 | O22190 |
| 77 | AtGH3-4 | Q9LQ68 |
| 78 | AtGH3-5 | O81829 |
| 79 | AtGH3-6 | Q9LSQ4 |
| 80 | CcGH3 | AAS02074 |
| 81 | GmGH3 | CAA42636 |
| 82 | MtGH3 | ABN09059 |
| 83 | NtGH3 | AAD32141 |
| 84 | PiGH3-16 | CAJ14972 |
| 85 | PtGH3-1 | EEE70316 |
| 86 | PtGH3-2 | EEE87842 |
| 87 | PtGH3-3 | EEE85053 |
| 88 | PtGH3-4 | EEE95321 |
| 89 | PtGH3-5 | EEE97566 |
| 90 | PtGH3-6 | EEE95183 |
| 91 | RcGH3-3 | XP_002524947 |
| 92 | RcGH3-6 | XP_002533739 |
| 93 | ThBAJ34606 | BAJ34606 |
| 94 | VvGH3-1 | XP_002271252 |
| 95 | VvGH3-2 | XP_002283886 |
| 96 | VvGH3-3 | XP_002283229 |
| 97 | VvGH3-5 | XP_002276241 |
| 98 | AtGH3-10 | AAD14468 |
| 99 | AtGH3-11 | AAD23040 |
| 100 | OsGH3-3 | EEE54131 |
| 101 | OsGH3-5 | BAG92652 |
| 102 | OsGH3-12 | Q53P49 |
| 103 | PtGH3-10 | EEE95291 |
| 104 | PtGH3-13 | EEE98498 |
| 105 | RcGH3-5 | EEF28472 |
| 106 | SbEES00046 | EES00046 |
| 107 | SbEES02233 | EES02233 |
| 108 | SbEES18777 | EES18777 |
| 109 | VvGH3-7 | XP_002272560 |
| 110 | VvGH3-9 | XP_002280738.1 |
| 111 | ZmACN26159 | ACN26159 |
| 112 | SlGH3.1 | SL2.40ch01: 78555183-78557768 |
| 113 | SlGH3.8 | Solyc07g054580.2 |
| 114 | SlGH3.14 | SGN-E745758 |
| 115 | GmGH3.2 | XP_003521802.1 |
| 116 | GmGH3.6 | XP_003540891.1 |
| 117 | GmGH3.9 | XP_003518806.1 |
| 118 | GmGH3.10 | XP_003541929.1 |
| 119 | GmGH3.14 | XP_003516612.1 |
| 120 | GmGH3.15 | XP_003540050.1 |
| 121 | GmGH3.24 | XP_003550026.1 |
| 122 | Gm03g30590.1 | Glyma03g30590.1 |
| 123 | BjACC38383.1 | ACC38383.1 |
| 124 | Br040953 | Bra040953 |
| 125 | Br016359 | Bra016359 |
| 126 | Br008836 | Bra008836 |
| 127 | Br006196 | Bra006196 |
| 128 | Br023405 | Bra023405 |
| 129 | Br040812 | Bra040812 |
| 130 | Br029173 | Bra029173 |
| 131 | Br031718 | Bra031718 |
| 132 | Br019920 | Bra019920 |
| 133 | Br000087 | Bra000087 |
| 134 | Sm87623 | Selmo1\|87623 |
| 135 | Sm93250 | Selmo1\|93250 |
| 136 | Sm85812 | Selmo1\|85812 |
| 137 | Sm416246 | Selmo1\|416246 |
| 138 | Sm233086 | Selmo1\|233086 |
| 139 | Sm437924 | XP_002961596 |
| 140 | Os05g05180.2 | Os05g05180.2 |
| 141 | HvBAJ85306.1 | BAJ85306.1 |
| 142 | Bd1g18200.1 | XP_003559815 |
| 143 | Br036998 | Bra036998 |
| 144 | GmGH3.22 | XP_003524670.1 |
| 145 | GmGH3.25 | XP_003550991.1 |

TABLE 4
Relative amount of conjugation products produced from GH3 protein
reactions

| | | | Relative amount of conjugates produced | | | |
|---|---|---|---|---|---|---|
| | | | from protein reaction | | | |
| | SEQ | | 2,4-D- | 2,4-D- | Dicamba- | |
| Gene name | ID | Subgroup | Asp | Glu | Glu | Dicamba-Asp |
| AIGH3-9 | 1 | II | − | + | − | − |
| AIGH3-17 | 2 | II | ++ | +++ | − | − |
| AtGH3-17 | 3 | II | +++ | +++ | − | − |
| AtGH3-9 | 4 | II | − | + | − | − |
| OsGH3-7 | 5 | II | − | + | − | − |
| OsGH3-11 | 6 | II | − | + | − | − |
| OsGH3-13 | 7 | II | +++ | +++++ | − | − |
| PtGH3-7 | 8 | II | +++ | +++++ | − | − |
| PtGH3-8 | 9 | II | − | ++ | − | − |
| PtGH3-9 | 10 | II | + | ++ | − | − |
| RcGH3-17 | 11 | II | − | + | − | − |
| SbEES08587 | 12 | II | + | ++ | − | − |
| VvGH3-4 | 13 | II | − | + | − | − |
| VvGH3-6 | 14 | II | ++ | +++ | − | − |
| PtGH3-14 | 15 | I | − | + | − | − |
| VvGH3-8 | 16 | I | − | + | − | − |
| OsGH3-6 | 17 | I | − | − | − | − |
| AIGH3-14 | 19 | III | + | ++ | − | − |
| AlGH3-12 | 22 | III | + | + | − | − |
| AlGH3-7 | 23 | III | − | − | − | − |
| AtGH3-12 | 24 | III | + | + | − | − |
| AtGH3-13 | 25 | III | − | + | − | − |
| AtGH3-14 | 26 | III | + | ++ | − | − |
| AtGH3-15 | 27 | III | − | − | − | − |
| AtGH3-19 | 30 | III | + | + | − | − |
| AtGH3-7 | 31 | III | − | − | − | − |
| OsGH3-1 | 33 | II | +++ | ++ | − | − |
| OsGH3-10 | 34 | II | − | − | − | − |
| OsGH3-2 | 35 | II | ++++ | +++ | − | − |
| OsGH3-4 | 36 | II | +++ | + | − | − |
| OsGH3-8 | 37 | II | ++++ | ++ | − | − |
| SbEER99639 | 40 | II | ++++ | +++ | − | − |
| SbEES01517 | 41 | II | +++ | +++ | − | − |
| SbEES01578 | 42 | II | ++ | + | − | − |
| SbEES16535 | 43 | II | − | − | − | − |
| SbEES19769 | 44 | II | +++++ | +++++ | − | − |
| ZmACF88044 | 45 | II | − | + | − | − |
| ZmACI46148 | 46 | II | ++++ | +++ | − | − |
| ZmACI46149 | 47 | II | +++++ | +++ | − | − |
| ZmACL52529 | 48 | II | − | − | − | − |
| ZmACL54563 | 49 | II | +++ | +++ | − | − |
| ZmACN32133 | 50 | II | +++ | ++ | − | − |
| PpGH3-1 | 51 | I | + | + | − | − |
| PpGH3-2 | 52 | I | +++ | +++++ | + | − |
| SmXP_002983845 | 57 | I | + | + | − | − |
| SmXP_002973755 | 58 | II | +++ | +++ | − | − |
| SmXP_002981880 | 62 | I | ++ | +++ | − | − |
| SmXP_002986992 | 66 | I | − | + | − | − |
| AIGH3-1 | 68 | II | ++++ | +++ | − | − |
| AIGH3-3 | 69 | II | ++ | + | − | − |
| AIGH3-4 | 70 | II | + | + | − | − |
| AIGH3-5 | 71 | II | ++++ | +++ | − | − |
| AIGH3-6 | 72 | II | +++ | ++ | − | − |
| AIGH3-2 | 73 | II | ++++ | +++ | − | − |
| AtGH3-1 | 74 | II | +++ | ++ | − | − |
| AtGH3-2 | 75 | II | +++ | + | − | − |
| AtGH3-3 | 76 | II | − | − | − | − |
| AtGH3-4 | 77 | II | ++++ | +++ | − | − |
| AtGH3-5 | 78 | II | ++++ | +++ | − | − |
| AtGH3-6 | 79 | II | ++++ | +++ | − | − |
| CcGH3 | 80 | II | ++++ | +++ | − | − |
| GmGH3-21 | 81 | II | ++++++ | ++++++ | − | − |
| MtGH3 | 82 | II | +++ | + | − | − |
| NtGH3 | 83 | II | ++++ | +++ | − | − |
| PiGH3-16 | 84 | II | +++ | + | − | − |
| PtGH3-1 | 85 | II | +++++ | ++ | − | − |
| PtGH3-2 | 86 | II | +++ | ++ | − | − |
| PtGH3-3 | 87 | II | ++++ | +++ | − | − |
| PtGH3-4 | 88 | II | +++ | +++ | − | − |
| PtGH3-5 | 89 | II | +++++ | +++ | − | − |
| PtGH3-6 | 90 | II | +++++ | +++++ | − | − |
| RcGH3-3 | 91 | II | +++++ | ++++ | − | − |
| RcGH3-6 | 92 | II | +++++ | ++++ | − | − |
| ThBAJ34606 | 93 | II | +++++ | ++++ | − | − |
| VvGH3-1 | 94 | II | ++++ | +++ | − | − |
| VvGH3-2 | 95 | II | +++++ | +++ | − | − |
| VvGH3-3 | 96 | II | +++++ | ++++ | − | − |
| VvGH3-5 | 97 | II | ++++ | +++ | − | − |
| Cell only control | N/A | N/A | − | − | − | − |
| Buffer control | N/A | N/A | − | − | − | − |

TABLE 5
Conjugase enzymatic activity (k_{cat}) of 78 tested GH3 proteins.

| | | | Protein | Protein | | | |
|---|---|---|---|---|---|---|---|
| | SEQ | Sub- | Purity | Conc. | Protein | k_{cat} (hrs−1) | |
| Gene name | ID | group | (%) | (μg/mL) | MW | IAA-Asp | IAA-Glu |
| AIGH3-9 | 1 | II | 30 | 92.75 | 67.38 | 0.000 | 0.000 |
| AIGH3-17 | 2 | II | 90 | 163.75 | 69.88 | 33.508 | 317.690 |
| AtGH3-17 | 3 | II | 65 | 95.25 | 69.77 | 51.838 | 867.723 |
| AtGH3-9 | 4 | II | 60 | 61.25 | 67.06 | 0.000 | 0.000 |
| OsGH3-7 | 5 | II | 20 | 51.25 | 69.85 | 0.000 | 0.000 |
| OsGH3-11 | 6 | II | 20 | 59.5 | 67.64 | 0.000 | 0.000 |
| OsGH3-13 | 7 | II | 70 | 153.5 | 72.76 | 10.812 | 494.321 |
| PtGH3-7 | 8 | II | 85 | 157.75 | 69.44 | 38.668 | 450.548 |
| PtGH3-8 | 9 | II | 30 | 68 | 67.46 | 13.999 | 568.780 |
| PtGH3-9 | 10 | II | 50 | 89.75 | 68.34 | 4.487 | 21.980 |
| RcGH3-17 | 11 | II | 35 | 82 | 69.1 | 4.366 | 10.433 |
| SbEES08587 | 12 | II | 35 | 77.25 | 73.58 | 10.251 | 389.161 |
| VvGH3-4 | 13 | II | 35 | 83.25 | 68.54 | 3.873 | 16.309 |
| VvGH3-6 | 14 | II | 50 | 98.5 | 69.77 | 111.443 | 505.272 |
| PtGH3-14 | 15 | I | 20 | 61.75 | 65.51 | 0.000 | 0.000 |
| VvGH3-8 | 16 | I | 35 | 70.25 | 65.06 | 0.000 | 0.000 |
| OsGH3-6 | 17 | I | 30 | 82.5 | 53.92 | 0.000 | 0.000 |
| AIGH3-14 | 19 | III | 70 | 89.25 | 68.01 | 0.000 | 1.756 |
| AIGH3-12 | 22 | III | 90 | 84.25 | 65.92 | 0.000 | 12.925 |
| AIGH3-7 | 23 | III | 30 | 80.5 | 55.44 | 0.000 | 0.000 |
| AtGH3-12 | 24 | III | 95 | 86.75 | 66.03 | 0.000 | 10.015 |
| AtGH3-13 | 25 | III | 60 | 73.25 | 67.28 | 0.000 | 0.000 |
| AtGH3-14 | 26 | III | 60 | 73.5 | 67.85 | 0.000 | 0.000 |
| AtGH3-15 | 27 | III | 50 | 86.5 | 65.27 | 0.000 | 0.000 |
| AtGH3-19 | 30 | III | 50 | 73 | 65.03 | 0.000 | 3.177 |
| AtGH3-7 | 31 | III | 40 | 91 | 66.52 | 0.000 | 5.361 |
| OsGH3-1 | 33 | II | 90 | 64.25 | 68.27 | 633.605 | 33.412 |
| OsGH3-10 | 34 | II | 10 | 52 | 53.11 | 0.000 | 0.000 |
| OsGH3-2 | 35 | II | 90 | 94 | 68.81 | 664.242 | 42.295 |
| OsGH3-4 | 36 | II | 30 | 78.5 | 69.69 | 919.336 | 17.854 |
| OsGH3-8 | 37 | II | 50 | 95.25 | 67.76 | 578.598 | 28.361 |
| SbEER99639 | 40 | II | 90 | 77.25 | 69.03 | 651.991 | 31.110 |
| SbEES01517 | 41 | II | 90 | 73.75 | 68.99 | 855.770 | 80.034 |
| SbEES01578 | 42 | II | 15 | 58.5 | 68.04 | 4419.692 | 162.572 |
| SbEES16535 | 43 | II | 20 | 56 | 71.83 | 0.000 | 0.000 |
| SbEES19769 | 44 | II | 50 | 84.5 | 71.1 | 903.124 | 50.990 |
| ZmACF88044 | 45 | II | 15 | 54.5 | 59.29 | 0.000 | 0.000 |
| ZmACI46148 | 46 | II | 90 | 67 | 68.35 | 869.016 | 49.496 |
| ZmACI46149 | 47 | II | 90 | 84 | 67.75 | 684.072 | 26.467 |
| ZmACL52529 | 48 | II | 50 | 92.75 | 60.64 | 0.000 | 2.149 |
| ZmACL54563 | 49 | II | 30 | 66.5 | 69.82 | 522.629 | 26.015 |
| ZmACN32133 | 50 | II | 90 | 74.25 | 67.81 | 876.059 | 83.547 |
| PpGH3-1 | 51 | I | 90 | 79.5 | 71.96 | 0.000 | 0.000 |
| PpGH3-2 | 52 | I | 90 | 99 | 67.91 | 2.896 | 19.308 |
| SmXP_002983845 | 57 | I | 85 | 75.5 | 65.65 | 0.000 | 0.000 |
| SmXP_002973755 | 58 | II | 85 | 84.75 | 68.02 | 220.320 | 9.285 |
| SmXP_002981880 | 62 | I | 85 | 78 | 63.37 | 1.475 | 1.759 |
| SmXP_002986992 | 66 | I | 60 | 108 | 66.25 | 0.000 | 0.000 |
| AIGH3-1 | 68 | II | 90 | 110.75 | 67.68 | 436.828 | 14.961 |
| AIGH3-3 | 69 | II | 5 | 67.25 | 68.39 | 3315.262 | 71.865 |
| AIGH3-4 | 70 | II | 85 | 84 | 67.94 | 3.013 | 0.000 |
| AIGH3-5 | 71 | II | 85 | 106 | 70.29 | 429.073 | 24.704 |
| AIGH3-6 | 72 | II | 85 | 126.5 | 69.8 | 413.293 | 20.881 |
| AIGH3-2 | 73 | II | 90 | 80.75 | 69.14 | 583.500 | 13.953 |
| AtGH3-1 | 74 | II | 90 | 85.75 | 67.56 | 437.707 | 19.230 |
| AtGH3-2 | 75 | II | 85 | 83 | 68.99 | 511.761 | 30.640 |
| AtGH3-3 | 76 | II | 20 | 91.75 | 68.44 | 0.000 | 0.000 |
| AtGH3-4 | 77 | II | 95 | 81 | 67.88 | 414.602 | 37.932 |
| AtGH3-5 | 78 | II | 90 | 84 | 70.19 | 578.727 | 68.086 |
| AtGH3-6 | 79 | II | 95 | 86 | 69.8 | 447.107 | 14.154 |
| CcGH3 | 80 | II | 90 | 110.5 | 68.28 | 414.234 | 15.608 |
| GmGH3-21 | 81 | II | 70 | 80.75 | 67.8 | 1087.519 | 1075.524 |
| MtGH3 | 82 | II | 70 | 77.25 | 69.65 | 337.033 | 11.549 |
| NtGH3 | 83 | II | 90 | 69.75 | 68.51 | 716.658 | 34.341 |
| PiGH3-16 | 84 | II | 90 | 66.25 | 70.77 | 656.761 | 51.037 |
| PtGH3-1 | 85 | II | 90 | 73.5 | 68.5 | 900.907 | 39.695 |
| PtGH3-2 | 86 | II | 90 | 67.75 | 68.31 | 679.646 | 53.401 |
| PtGH3-3 | 87 | II | 90 | 52 | 68.61 | 806.314 | 44.225 |
| PtGH3-4 | 88 | II | 70 | 58.75 | 69.7 | 660.985 | 93.781 |
| PtGH3-5 | 89 | II | 95 | 54 | 69.97 | 941.117 | 160.490 |
| PtGH3-6 | 90 | II | 90 | 59.75 | 69.89 | 888.111 | 181.954 |
| RcGH3-3 | 91 | II | 85 | 71 | 68.35 | 887.172 | 36.015 |
| RcGH3-6 | 92 | II | 90 | 44.25 | 70 | 1037.037 | 183.972 |
| ThBAJ34606 | 93 | II | 85 | 72.25 | 70.25 | 610.082 | 67.109 |
| VvGH3-1 | 94 | II | 85 | 56 | 68.15 | 964.027 | 45.433 |
| VvGH3-2 | 95 | II | 85 | 52.75 | 68.35 | 1011.181 | 45.833 |
| VvGH3-3 | 96 | II | 80 | 47.75 | 70.32 | 1006.325 | 79.770 |
| VvGH3-5 | 97 | II | 80 | 61 | 69.93 | 759.486 | 47.289 |

Conjugase enzymatic activity (kcat) of 78 tested GH3 proteins (continued).

| | | | Protein | Protein | | k_{cat} (hrs−1) | |
|---|---|---|---|---|---|---|---|
| | SEQ | Sub- | Purity | Conc. | Protein | 2,4-D- | 2,4-D- |
| Gene name | ID | group | (%) | (μg/mL) | MW | Asp | Glu |
| AIGH3-9 | 1 | II | 30 | 92.75 | 67.38 | 0.000 | 0.015 |
| AIGH3-17 | 2 | II | 90 | 163.75 | 69.88 | 0.212 | 0.455 |
| AtGH3-17 | 3 | II | 65 | 95.25 | 69.77 | 0.424 | 0.988 |
| AtGH3-9 | 4 | II | 60 | 61.25 | 67.06 | 0.000 | 0.005 |
| OsGH3-7 | 5 | II | 20 | 51.25 | 69.85 | 0.000 | 0.028 |
| OsGH3-11 | 6 | II | 20 | 59.5 | 67.64 | 0.000 | 0.043 |
| OsGH3-13 | 7 | II | 70 | 153.5 | 72.76 | 0.639 | 5.237 |
| PtGH3-7 | 8 | II | 85 | 157.75 | 69.44 | 0.547 | 3.487 |
| PtGH3-8 | 9 | II | 30 | 68 | 67.46 | 0.000 | 0.559 |
| PtGH3-9 | 10 | II | 50 | 89.75 | 68.34 | 0.065 | 0.305 |
| RcGH3-17 | 11 | II | 35 | 82 | 69.1 | 0.000 | 0.027 |
| SbEES08587 | 12 | II | 35 | 77.25 | 73.58 | 0.044 | 0.554 |
| VvGH3-4 | 13 | II | 35 | 83.25 | 68.54 | 0.000 | 0.018 |
| VvGH3-6 | 14 | II | 50 | 98.5 | 69.77 | 0.385 | 1.204 |
| PtGH3-14 | 15 | I | 20 | 61.75 | 65.51 | 0.000 | 0.015 |
| VvGH3-8 | 16 | I | 35 | 70.25 | 65.06 | 0.000 | 0.028 |
| OsGH3-6 | 17 | I | 30 | 82.5 | 53.92 | 0.000 | 0.000 |
| AIGH3-14 | 19 | III | 70 | 89.25 | 68.01 | 0.140 | 0.322 |
| AIGH3-12 | 22 | III | 90 | 84.25 | 65.92 | 0.006 | 0.057 |
| AIGH3-7 | 23 | III | 30 | 80.5 | 55.44 | 0.000 | 0.000 |
| AtGH3-12 | 24 | III | 95 | 86.75 | 66.03 | 0.002 | 0.026 |
| AtGH3-13 | 25 | III | 60 | 73.25 | 67.28 | 0.000 | 0.018 |
| AtGH3-14 | 26 | III | 60 | 73.5 | 67.85 | 0.122 | 0.233 |
| AtGH3-15 | 27 | III | 50 | 86.5 | 65.27 | 0.000 | 0.000 |
| AtGH3-19 | 30 | III | 50 | 73 | 65.03 | 0.014 | 0.048 |
| AtGH3-7 | 31 | III | 40 | 91 | 66.52 | 0.000 | 0.000 |
| OsGH3-1 | 33 | II | 90 | 64.25 | 68.27 | 0.937 | 0.207 |
| OsGH3-10 | 34 | II | 10 | 52 | 53.11 | 0.000 | 0.000 |
| OsGH3-2 | 35 | II | 90 | 94 | 68.81 | 1.548 | 0.401 |
| OsGH3-4 | 36 | II | 30 | 78.5 | 69.69 | 2.002 | 0.254 |
| OsGH3-8 | 37 | II | 50 | 95.25 | 67.76 | 2.917 | 0.368 |
| SbEER99639 | 40 | II | 90 | 77.25 | 69.03 | 2.476 | 0.457 |
| SbEES01517 | 41 | II | 90 | 73.75 | 68.99 | 0.953 | 0.426 |
| SbEES01578 | 42 | II | 15 | 58.5 | 68.04 | 0.765 | 0.250 |
| SbEES16535 | 43 | II | 20 | 56 | 71.83 | 0.000 | 0.000 |
| SbEES19769 | 44 | II | 50 | 84.5 | 71.1 | 18.287 | 4.970 |
| ZmACF88044 | 45 | II | 15 | 54.5 | 59.29 | 0.000 | 0.033 |
| ZmACI46148 | 46 | II | 90 | 67 | 68.35 | 2.278 | 0.401 |
| ZmACI46149 | 47 | II | 90 | 84 | 67.75 | 2.892 | 0.355 |
| ZmACL52529 | 48 | II | 50 | 92.75 | 60.64 | 0.000 | 0.000 |
| ZmACL54563 | 49 | II | 30 | 66.5 | 69.82 | 3.255 | 0.993 |
| ZmACN32133 | 50 | II | 90 | 74.25 | 67.81 | 0.602 | 0.241 |
| PpGH3-1 | 51 | I | 90 | 79.5 | 71.96 | 0.078 | 0.122 |
| PpGH3-2 | 52 | I | 90 | 99 | 67.91 | 0.699 | 2.820 |
| SmXP_002983845 | 57 | I | 85 | 75.5 | 65.65 | 0.090 | 0.052 |
| SmXP_002973755 | 58 | II | 85 | 84.75 | 68.02 | 1.076 | 0.431 |
| SmXP_002981880 | 62 | I | 85 | 78 | 63.37 | 0.144 | 0.739 |
| SmXP_002986992 | 66 | I | 60 | 108 | 66.25 | 0.000 | 0.006 |
| AIGH3-1 | 68 | II | 90 | 110.75 | 67.68 | 2.057 | 0.525 |
| AIGH3-3 | 69 | II | 5 | 67.25 | 68.39 | 1.566 | 0.172 |
| AIGH3-4 | 70 | II | 85 | 84 | 67.94 | 0.040 | 0.006 |
| AIGH3-5 | 71 | II | 85 | 106 | 70.29 | 1.586 | 0.421 |
| AIGH3-6 | 72 | II | 85 | 126.5 | 69.8 | 1.300 | 0.211 |
| AIGH3-2 | 73 | II | 90 | 80.75 | 69.14 | 1.903 | 0.309 |
| AtGH3-1 | 74 | II | 90 | 85.75 | 67.56 | 1.188 | 0.136 |
| AtGH3-2 | 75 | II | 85 | 83 | 68.99 | 1.200 | 0.131 |
| AtGH3-3 | 76 | II | 20 | 91.75 | 68.44 | 0.000 | 0.000 |
| AtGH3-4 | 77 | II | 95 | 81 | 67.88 | 2.032 | 0.594 |
| AtGH3-5 | 78 | II | 90 | 84 | 70.19 | 2.309 | 0.795 |
| AtGH3-6 | 79 | II | 95 | 86 | 69.8 | 2.136 | 0.598 |
| CcGH3 | 80 | II | 90 | 110.5 | 68.28 | 2.517 | 0.577 |
| GmGH3-21 | 81 | II | 70 | 80.75 | 67.8 | 27.468 | 22.910 |
| MtGH3 | 82 | II | 70 | 77.25 | 69.65 | 1.494 | 0.122 |
| NtGH3 | 83 | II | 90 | 69.75 | 68.51 | 1.990 | 0.804 |
| PiGH3-16 | 84 | II | 90 | 66.25 | 70.77 | 0.379 | 0.089 |
| PtGH3-1 | 85 | II | 90 | 73.5 | 68.5 | 4.798 | 0.181 |
| PtGH3-2 | 86 | II | 90 | 67.75 | 68.31 | 0.934 | 0.203 |
| PtGH3-3 | 87 | II | 90 | 52 | 68.61 | 2.033 | 0.845 |
| PtGH3-4 | 88 | II | 70 | 58.75 | 69.7 | 0.740 | 0.536 |
| PtGH3-5 | 89 | II | 95 | 54 | 69.97 | 3.142 | 1.214 |
| PtGH3-6 | 90 | II | 90 | 59.75 | 69.89 | 5.415 | 2.816 |
| RcGH3-3 | 91 | II | 85 | 71 | 68.35 | 3.813 | 1.854 |
| RcGH3-6 | 92 | II | 90 | 44.25 | 70 | 4.189 | 2.525 |
| ThBAJ34606 | 93 | II | 85 | 72.25 | 70.25 | 4.232 | 1.750 |
| VvGH3-1 | 94 | II | 85 | 56 | 68.15 | 2.529 | 0.635 |
| VvGH3-2 | 95 | II | 85 | 52.75 | 68.35 | 5.691 | 1.113 |
| VvGH3-3 | 96 | II | 80 | 47.75 | 70.32 | 5.615 | 1.602 |
| VvGH3-5 | 97 | II | 80 | 61 | 69.93 | 2.603 | 0.974 |

Conjugase enzymatic activity (k_{cat}) of tested GH3 proteins (continued).

| | | | Protein | | |
|---|---|---|---|---|---|
| | SEQ | | purity | Protein conc. | k_{cat} (16 hrs⁻¹) |
| Gene name | ID | Subgroup | (%) | (μg/mL) | dicamba-Glu |
| PpGH3-2* | 52 | I | 90 | 983 | 0.024 |

*Data not available for dicamba-Glu conjugase activity with other genes.

TABLE 6
Conjugation activity of additional GH3 proteins.

| | | | Detected auxin conjugates | | | | | |
|---|---|---|---|---|---|---|---|---|
| | SEQ | Sub- | IAA- | IAA- | 2,4-D- | 2,4-D- | Dicamba- | Dicamba- |
| Gene name | ID | group | Asp | Glu | Asp | Glu | Asp | Glu |
| AtGH3-11 | 99 | I | No | No | No | No | No | No |
| OsGH3-3 | 100 | I | Yes | No | No | No | No | No |
| OsGH3-12 | 102 | I | No | No | No | No | No | No |
| PtGH3-10 | 103 | I | Yes | Yes | Yes | Yes | No | No |
| RcGH3-5 | 105 | I | Yes | No | No | No | No | No |
| VvGH3-7 | 109 | I | Yes | No | No | No | No | No |
| VvGH3-9 | 110 | I | No | No | No | No | No | No |
| ZmACN26159 | 111 | I | No | No | No | No | No | No |
| SlGH3-1 | 112 | I | Yes | No | No | No | No | No |
| SlGH3-8 | 113 | I | Yes | Yes | Yes | Yes | No | No |
| SlGH3-14 | 114 | I | No | No | No | No | No | No |
| GmGH3-2 | 115 | I | Yes | No | No | No | No | No |
| GmGH3-6 | 116 | I | Yes | Yes | No | No | No | No |
| GmGH3-10 | 118 | II | Yes | Yes | Yes | Yes | No | No |
| GmGH3-14 | 119 | II | No | No | No | No | No | No |
| GmGH3-15 | 120 | II | Yes | Yes | Yes | Yes | No | No |
| Gm03g30590.1 | 122 | I | Yes | No | Yes | Yes | No | No |
| BjACC38383.1 | 123 | III | Yes | No | No | No | No | No |
| Br040953 | 124 | II | Yes | Yes | Yes | Yes | No | No |
| Br016359 | 125 | III | Yes | No | Yes | No | No | No |
| Br008836 | 126 | III | Yes | Yes | Yes | Yes | No | No |
| Br040812 | 129 | III | Yes | Yes | Yes | Yes | No | No |
| Br029173 | 130 | III | Yes | Yes | Yes | Yes | No | No |
| Br031718 | 131 | III | No | No | No | No | No | No |
| Sm87623 | 134 | I | Yes | No | Yes | No | No | No |
| Sm93250 | 135 | I | Yes | No | No | No | No | No |
| Sm85812 | 136 | I | Yes | No | No | No | No | No |

### Example 5

### Transformation of Arabidopsis with GH3 Genes and Evaluation of Herbicide Response

Arabidopsis (Arabidopsis thaliana) expressing GH3 genes were produced using floral dip methods of Agrobacterium mediated transformation (Clough S J and Bent A F, 1998, Plant J. 16:735-43; Chung M. H., Chen M. K., Pan S. M. 2000. Transgenic Res. 9: 471-476; Weigel D. and Glazebrook J. 2006. In Planta Transformation of Arabidopsis. Cold Spring Harb. Protoc. 4668 3). Briefly, Arabidopsis (Col-0) plants were grown in soil in pots. The first inflorescence shoots were removed as soon as they emerged. Plants were ready for transformation when the secondary inflorescence shoots were about 3 inches tall. Agrobacterium carrying a suitable binary vector were cultured in 5 mL LB medium at 28° C. with shaking at 200 rpm for two days. 1 mL of the culture was then inoculated into 200 ml fresh LB media and incubated again with vigorous agitation for an additional 20-24 hours at 28° C. The Agrobacterium culture was then subjected to centrifugation at 6000 rpm in a GSA rotor (or equivalent) for 10 minutes. The pellet was resuspended in 20-100 mL of spraying medium containing 5% (wt/v) sucrose and 0.01-0.2% (v/v) Silwet L-77. The Agrobacterium suspension was transferred into a hand-held sprayer for spraying onto inflorescences of the transformation-ready Arabidopsis plants. The sprayed plants were covered with a humidity dome for 24 hours before the cover was removed for growth under normal growing conditions. Seeds were harvested. Screening of transformants was performed under sterile conditions. Surface sterilized seeds were placed onto MS-Agar plates (Phyto Technology labs Prod. No. M519) containing appropriate selective antibiotics (kanamycin 50 mg/L, hygromycin 20 mg/L, or bialaphos 10 mg/L). Anti-Agrobacteirum antibiotic timentin was also included in the media. Plates were cultured at 21° C. at 16 hr light for 7-14 days. Transgenic events harboring GH3 genes were germinated and transferred to soil pots in the greenhouse for evaluation of herbicide tolerance.

A selectable marker gene used to facilitate Arabidopsis transformation was a chimeric gene composed of the 35S promoter from Cauliflower Mosaic Virus (Odell et al. 1985. Nature 313:810-812), the bar gene from Streptomyces hygroscopicus (Thompson et al., 1987. EMBO J. 6:2519-2523), and the 3′UBQ14 terminator region from Arabidopsis (Callis et al., 1995. Genetics 139 (2), 921-939). Another visual selectable marker gene used to facilitate Arabidopsis transformation was a chimeric gene composed of the UBQ promoter from soybean (Xing et al., 2010. Plant Biotechnology Journal 8:772-782), the YFP coding sequence, and the 3′ region of the nopaline synthase gene from the T-DNA of the Ti plasmid of Agrobacterium tumefaciens. Bialophos was used as the selection agent during the transformation process. GH3 genes were expressed with a constitutive promoter, for example, the Arabidopsis UBQ10 promoter (Norris et al., 1993. Plant Mol Biol 21:895-906) or UBQ3 promoter (Norris et al., 1993. Plant Mol Biol 21:895-906) for strong or moderate expression, a GH3 coding sequence, and the 3′ terminator region of the French bean phaseolin gene (Sun et al., 1981. Nature 289:37-41; Slightom et al., 1983. Proc. Natl. Acad. Sci. U.S.A. 80 (7), 1897-1901)

Seeds of Arabidopsis ecotype Columbia (Col-0) and GH3 transgenic events were surface sterilized with 70% (v/v) ethanol for 5 minutes and 10% (v/v) bleach for 15 minutes. After being washed three times with distilled water, the seeds were incubated at 4° C. for 4 days. The seeds were then germinated on 1× Murashige and Skoog (MS) medium with a pH of 5.7, 3% (w/v) sucrose, and 0.8% (w/v) agar. After incubation for 3.5 days, the seedlings were transferred to the basal medium containing B5 vitamin, 3% (w/v) sucrose, 2.5 mm MES (pH 5.7), 1.2% (w/v) agar, and filter sterilized auxin herbicides 2,4-D or dicamba was added to the media at 60° C. The concentrations of 2,4-D were 0 μM, 0.1 μM, 0.5 μM, 0.7 μM, and 1.0 μM. The concentrations of dicmaba were 0 μM, 1.0 μM, 5.0 μM, 7.0 μM, and 10 μM. The basal medium contained 1/10×MS macronutrients (2.05 mm NH₄NO₃, 1.8 mm KNO₃, 0.3 mm CaCl₂, and 0.156 mm MgSO₄) and 1×MS micronutrients (100 μm H₃BO₃, 100 μm MnSO₄, 30 μm ZnSO₄, 5 μm KI, 1 μm Na₂MoO₄, 0.1 μm CuSO₄, 0.1 μm CoCl₂, 0.1 mm FeSO₄, and 0.1 mm Na₂EDTA). The seedlings were placed vertically, and the temperature maintained at 23° C. to allow root growth along the surface of the agar, with a photoperiod of 16 h of light and 8 h of dark.

After 6 days on media with various concentrations of 2,4-D or dicamba, the length of the primary root was measured. In wild type Arabidopsis, root growth inhibition is expected from auxin herbicide treatment. The length of the primary root in wild type plants is reduced with 2,4-D or dicamba treatment. The more 2,4-D or dicamba, the shorter the primary root. The difference in root growth inhibition between wild type and GH3 transgenic events is compared. Alleviation of root growth inhibition on 2,4-D or dicamba is an indication of auxin herbicide detoxification due to GH3 conjugation activity.

### Example 6

### Transformation of Soybean with GH3 Genes

Soybean plants expressing GH3 transgenes were produced using the method of particle gun bombardment (Klein et al. (1987) Nature 327:70-73, U.S. Pat. No. 4,945,050) using a DuPont Biolistic PDS1000/He instrument. GH3 transgenes include coding sequences of active 2,4-D or dicamba conjugases such as PtGH3-1 (SEQ ID NO:85), PtGH3-7 (SEQ ID NO:8), and RcGH3-6 (SEQ ID NO:92). A selectable marker gene used to facilitate soybean transformation was a chimeric gene composed of the 35S promoter from Cauliflower Mosaic Virus (Odell et al. (1985) Nature 313:810-812), the hygromycin phosphotransferase gene from plasmid pJR225 (from E coli; Gritz et al. (1983) Gene 25:179-188) and the 3′ region of the nopaline synthase gene from the T-DNA of the Ti plasmid of Agrobacterium tumefaciens. Another selectable marker used to facilitate soybean transformation was a chimeric gene composed of the S-adenosylmethionine synthase (SAMS) promoter (U.S. Pat. No. 7,741,537) from soybean, a highly resistant allele of ALS (U.S. Pat. Nos. 5,605,011, 5,378,824, 5,141,870, and 5,013,659), and the native soybean ALS terminator region. The selection agent used during the transformation process was either hygromycin or chlorsulfuron depending on the marker gene present. GH3 genes were expressed with a constitutive promoter, for example, the Arabidopsis UBQ10 promoter (Norris et al. (1993) Plant Mol Biol 21:895-906), a GH3 polypeptide, and the phaseolin gene terminator (Sun S M et al. (1981) Nature 289:37-41 and Slightom et al. (1983) Proc. Natl. Acad. Sci. U.S.A. 80 (7), 1897-1901). Bombardments were carried out with linear DNA fragments purified away from any bacterial vector DNA. The selectable marker gene cassette was in the same DNA fragment as the GH3 cassette. Bombarded soybean embryogenic suspension tissue was cultured for one week in the absence of selection agent, and then placed in liquid selection medium for 6 weeks. Putative transgenic suspension tissue was sampled for PCR analysis to determine the presence of the GH3 gene. Putative transgenic suspension culture tissue was maintained in selection medium for 3 weeks to obtain enough tissue for plant regeneration. Suspension tissue was matured for 4 weeks using standard procedures; matured somatic embryos were desiccated for 4-7 days and then placed on germination induction medium for 2-4 weeks. Germinated plantlets were transferred to soil in cell pack trays for 3 weeks for acclimatization. Plantlets were potted to 10-inch pots in the greenhouse for evaluation of herbicide resistance. Transgenic soybean, Arabidopsis and other species of plants could also be produced using Agrobacterium transformation using a variety of ex-plants.

### Example 7

### Herbicide Tolerance Evaluation of GH3 Transgenic Soybean Plants

T0 plants expressing GH3 transgenes are grown in a controlled environment (for example, 25° C., 70% humidity, 16 hr light) to either V2 or V8 growth stage and then sprayed with commercial 2,4-D or dicamba herbicide formulations at a rate up to 450 g/ha. Herbicide applications may be made with added 0.25% nonionic surfactant and 1% ammonium sulfate in a spray volume of 374 L/ha. Individual plants are compared to untreated plants of similar genetic background, evaluated for herbicide response at seven to twenty-one days after treatment and assigned a visual response score from 0 to 100% injury (0=no effect to 100=dead plant). Expression of the GH3 gene varies due to the genomic location in the unique T0 plants. Plants that do not express the transgenic GH3 gene are severely injured by 2,4-D or dicamba herbicide. T0 plants expressing introduced GH3 genes may show tolerance to the 2,4-D or dicamba herbicide due to activity of the GH3 protein.

Alternatively, evaluation of herbicide tolerance is performed through analysis of conjugation products. Seeds of GH3 transgenic soybean plants are surface sterilized and germinated on GB-5 basal media supplemented with 2,4-D or dicamba as described in Example 1. Ten days after germination, GH3 transgenic seeds are morphologically examined and compared with non-transgenic seeds. Non-transgenic seeds do not tolerate 2,4-D or dicamba in the media at concentration as low as 1 μM and thus do not germinate and grow well. Transgenic seeds with tolerance to 2,4-D or dicamba germinate and grow well with roots well-extended and true leaves emerged. Tissues of the germinated seeds are collected and ground using a mortar and pestle in extraction buffer containing 100 mM potassium phosphate (pH 7.0), 5 mM MgSO₄, and 1 mM DTT. Total extractions are subject to centrifugation at 14,000 rpm 4° C., for 30 min, supernatant is transferred and mixed with ice-cold methanol to reach a final concentration of methanol of 90%. The mixture is centrifuged again for 30 min at 14,000 rpm. Supernatant is then diluted in ddH₂O to reach final methanol concentration at 5% and subjected to LC-MS analysis. Higher level of 2,4-D-Glu or 2,4-D-Asp or dicamba-Glu or dicamba-Asp detected in samples prepared from GH3 transgenic tissue compared with that from non-transgenic tissue indicates more efficient detoxification of auxin herbicides into non-herbicidal Asp- or Glu-conjugates by transgenic GH3 enzymes in soybean plants.

Activity of soybean transgenic GH3 proteins are also evaluated using plant extracts. Tissues of GH3 transgenic plants treated are collected and ground in CCLR buffer containing 100 mM potassium phosphate (pH 7.8), 1 mM EDTA, 7 mM β-mercaptoethanol, 1% Triton, and 10% glycerol to obtain total protein extract. After centrifugation at 14,000 rpm at 4° C. for 30 min, the supernatant is transferred to conjugation reaction buffer containing 100 mM potassium phosphate (pH 7.0), 5 mM MgSO₄, 1 mM DTT, and 5 mM 2,4-D or dicamba. The reaction is carried out at room temperature and samples taken at various time points are mixed with ice-cold methanol to reach the final methanol concentration of 90%. The mixture is subject to centrifugation again for 30 min at 14,000 rpm. Supernatant is then diluted in ddH₂O to reach final methanol concentration of 5% and subjected to LC-MS analysis. Higher level of 2,4-D-Glu or 2,4-D-Asp or dicamba-Glu or dicamba-Asp detected in samples prepared from GH3 transgenic tissue compared with that from non-transgenic tissue indicates more efficient detoxification of auxin herbicides into non-herbicidal Asp- or Glu-conjugates by transgenic GH3 enzymes in soybean plants. The same protocols can be used with Arabidopsis and other plants expressing introduced GH3 gene variants.

### Example 8

### Example of 2,4-D Tolerance in Transgenic Arabidopsis Overexpressing GH3 Auxin Conjugating Enzyme

Transgenic Arabidopsis lines overexpressing SEQ ID NO:3 were generated and evaluated for 2,4-D herbicide response as described in Example 5. The Arabidopsis UBQ10 promoter was used to drive the expression of SEQ ID NO:3. Table 7 shows alleviation of root growth inhibition by 2,4-D in Arabidopsis T2 transgenic plants (8 plants; segregation in this generation yielded plants in this group which were homozygous and heterozygous) overexpressing SEQ ID NO:3. Root length of 8 plants per transgenic line was measured and the average was compared to untreated seedlings within the same genetic background. Root growth inhibition is expressed as percent reduction of root length in plants grown on 2,4-D. In wild-type Col-0 Arabidopsis, the primary root length was reduced to 77% when grown on 10 nM 2,4-D. Transgenic lines overexpressing SEQ ID NO:3 showed various degree of alleviation of root growth inhibition when grown on 10 nM 2,4-D, having smaller reduction in root length compared with wild-type (Table 7). In transgenic line #4, the inhibition of root growth by 2,4-D was completely alleviated at 10 nM 2,4-D.

TABLE 7
Inhibition of root growth by 2,4-D in wild-type
(Col-0) and transgenic Arabidopsis plants.

| Transgenic Lines | | | |
|---|---|---|---|
| Overexpressing | 2,4-D concentration | | |
| SEQ ID NO: 3 | 0 nM | 10 nM | 100 nM |
| Line #1 | 100* | 80 | 43 |
| Line #2 | 100 | 83 | 36 |
| Line #3 | 100 | 88 | 63 |
| Line #4 | 100 | 101 | 60 |
| Line #5 | 100 | 76 | 50 |
| Non transgenic | 100 | 77 | 58 |

*Percentage of the average root length of plants in the same genetic background when compared with 0 nM 2,4-D treatment.

Prospectively, the above techniques can be used to generate transgenic Arabidopsis and evaluated for 2,4-D herbicide, or alternatively dicamba, response as described in Example 5. For example, Arabidopsis UBQ10 promoter can be used to drive the expression of SEQ ID #26, #52, #57, #81, and #121. Alleviation of root growth inhibition by 2,4-D can be determined in Arabidopsis T2 transgenic plants (screening and selection for homozygous plants) overexpressing SEQ ID #26, #52, #57, and #81. Root length of 8 plants per transgenic line will be measured and the average can be compared to untreated seedlings within the same genetic background.

### Example 9

### Example of Soybean Seedling Response to Applied Auxin Conjugates

Conjugate forms of auxinic compounds when applied through spot infiltration, a needleless method to introduce exogenous agents into plant cells. Initially, various concentrations of analytical grade (98%+pure) IAA, 2,4-D, and dicamba were prepared as solutions that were applied directly to uni-foliate leaves of 9 day old soybean seedlings by spot infiltration in order to determine the range of auxinic response and severity.

Spot infiltration comprises applying a compound to be tested in composition that is applied to the surface of a plant, for example, to a leaf or other plant structure. For example, a method for testing a plant response to one or more compounds using spot infiltration comprises a) providing a composition comprising one or more compounds to be tested in a container having at least an opening for the compound enter and/or exit, and one or more elements for moving the composition into or out of the container; b) contacting at least one opening of the container with the surface of a plant and moving the composition comprising one or more compounds to at least the surface of the plant. A container and elements for moving the composition may comprise a syringe body and plunger. Moving the composition to the plant surface comprises contacting the surface with pressure sufficient for the composition to enter the cells of the plant. In an aspect, spot filtration testing comprises measuring an effect of the composition on the contacted plant and comparing that measure or effect to a plant that is not contacted with the composition.

Plant response phenotypes were documented at one day post-spot infiltration and subsequently every other day, up to nine days post treatment. Three concentrations were chosen based on reproducibility. In the studies described herein below, 5 replicate treatments for each conjugate form were conducted, along with appropriate negative controls. Data are summarized in Table 8 below.

Soybean plants spot infiltrated with IAA in 1% MeOH at concentrations of 1, 2.5 and 5 mM displayed modest stem twisting and bending (epinasty) at 1 day post treatment with increasing severity over the test range. No discernable phenotype was observed in seedlings treated with conjugate forms IAA-Asp, IAA-Glu or IAA-Phe at all test levels. Spot infiltration of IAA-Phe, dissolved in 50% EtOH, produced a necrotic lesion on leaf surface at point of application consistent with observation of 50% EtOH alone control.

Soybean plants spot infiltrated with dicamba in 5% EtOH at concentrations of 50, 100 and 250 μM reproducibly displayed epinasty at day 1 and leaf curling of 1^{st} tri-foliate, at all doses, within 2-4 days post treatment. Plants spot infiltrated with higher concentration of dicamba displayed more pronounced auxinic phenotype. Plants spot infiltrated with dicamba-Asp or dicamba-Glu showed phenotypes similar to that of negative control or plants infiltrated with 5% EtOH. No obvious auxinic phenotype was observed with these plants.

Soybean plants spot infiltrated with 2,4-D in 5% EtOH at concentrations of 100,250 & 500 μM displayed increased auxinic response ranging from mild to extreme epinasty respective of amount applied. Relative to dicamba, a much lesser extent of leaf curling at the first trifoliate was observed; however progressive chlorosis stemming from solution application points was displayed for all 2,4-D and conjugate forms. Such response was not observed in the negative control. Plants spot infiltrated with 2,4-D-Asp showed auxinic response nearly equivalent to that of 2,4-D, with similar extent of epinasty at the respective concentrations. Similarly, 2,4-D-Phe treated plants displayed similar degree of epinasty to that 2,4-D at respective levels. A distinctly lower level of severity was observed on plants spot infiltrated with 2,4-D-Glu, only at 500 μM did modest epinasty occur. Plants spot infiltrated with lower concentration of 2,4-D-Glu showed no discernible auxinic responses.

TABLE 8
Soybean seedling response to applied auxin conjugates.

| | | | | | | New |
|---|---|---|---|---|---|---|
| | | | | 1st | Chlorotic | growth |
| | | | Epinasty | trifoliate | lesion @ | recov- |
| Auxin | Conc. | | score | leaf curl | app. Pnt | ery |
| IAA | 1.0 | mM | + | 0 | 0 | + |
| IAA | 2.5 | mM | + | 0 | 0 | + |
| IAA | 5.0 | mM | ++ | 0 | 0 | + |
| IAA-Asp | 1.0 | mM | 0 | 0 | 0 | + |
| IAA-Asp | 2.5 | mM | 0 | 0 | 0 | + |
| IAA-Asp | 5.0 | mM | 0 | 0 | 0 | + |
| IAA-Glu | 1.0 | mM | 0 | 0 | 0 | + |
| IAA-Glu | 2.5 | mM | 0 | 0 | 0 | + |
| IAA-Glu | 5.0 | mM | 0 | 0 | 0 | + |
| IAA-Phe | 1.0 | mM | 0 | 0 | 0 | + |
| IAA-Phe | 2.5 | mM | 0 | 0 | 0 | + |
| IAA-Phe | 5.0 | mM | 0 | 0 | 0 | + |
| dicamba | 50 | μM | +++ | + | 0 | − |
| dicamba | 100 | μM | ++++ | + | 0 | − |
| dicamba | 250 | μM | +++++ | + | 0 | − |
| dicamba-Asp | 50 | μM | 0 | 0 | 0 | + |
| dicamba-Asp | 100 | μM | 0 | 0 | 0 | + |
| dicamba-Asp | 250 | μM | 0 | 0 | 0 | + |
| dicamba-Glu | 50 | μM | 0 | 0 | 0 | + |
| dicamba-Glu | 100 | μM | 0 | 0 | 0 | + |
| dicamba-Glu | 250 | μM | 0 | 0 | 0 | + |
| 2,4-D | 100 | μM | ++ | 0 | + | + |
| 2,4-D | 250 | μM | +++ | 0 | + | + |
| 2,4-D | 500 | μM | ++++ | 0 | + | + |
| 2,4-D-Asp | 100 | μM | ++ | 0 | + | + |
| 2,4-D-Asp | 250 | μM | +++ | 0 | + | + |
| 2,4-D-Asp | 500 | μM | ++++ | 0 | + | + |
| 2,4-D-Glu | 100 | μM | +/− | 0 | + | + |
| 2,4-D-Glu | 250 | μM | +/− | 0 | + | + |
| 2,4-D-Glu | 500 | μM | ++ | 0 | + | + |
| 2,4-D-Phe | 100 | μM | ++ | 0 | + | + |
| 2,4-D-Phe | 250 | μM | +++ | 0 | + | + |
| 2,4-D-Phe | 500 | μM | ++++ | 0 | + | + |
| H₂0 | | | 0 | 0 | 0 | control |
| 1% MeOH | | | 0 | 0 | 0 | control |
| 10% EtOH | | | 0 | 0 | 0 | control |
| 50% EtOH | | | 0 | 0 | + | control |

0 = no phenotype
+/− = threshold

### Example 10

### Example of Arabidopsis Response to Applied Auxin Conjugates

The effect of auxin conjugates was assessed using Arabidoposis (Arabidopsis thialiana) ecotype Col-0 plant. Prior to studies utilizing the auxin conjugates, levels of analytical grade (98%+pure) IAA, 2,4-D and dicamba in solution were applied at various concentrations directly to developmentally similar leaves of 20-23 day old Arabidopsis seedlings by needleless syringe spot infiltration methodology, in order to determine range of auxinic response and severity. Phenotypes were documented relative to control at 1 day post-spot infiltration and subsequently every other day, up to nine days post treatment. Three concentrations were chosen based on reproducibility, and conjugated forms were then studied with 4 or more replicate treatments for each, along with appropriate negative controls (H₂O and 10% EtOH).

IAA (dissolved in 5% EtOH) applied to (2) developmentally similar leaves of 21 day plants at concentrations of 1, 2.5 and 5 mM, display modest petiole twisting (epinasty) and leaf curl at 1-2 days post treatment at 5 mM (high dose). No discernable phenotype was observed in plants treated with conjugate forms IAA-Asp or IAA-Glu at all test levels.

Dicamba free acid (dissolved in 5% EtOH) applied at concentrations of 50, 100 and 250 μM reproducibly displayed petiole epinasty and leaf curling at day one, with the most pronounced severity at 250 μM. No obvious phenotype was observed with conjugated dicamba-Asp treatment performed at same time and levels; appearing equivalent to negative control. Dicamba-Glu displayed modest epinasty, compared to dicamba alone, at 100 and 250 μM levels.

2,4-D (dissolved in 5% EtOH) applied by spot infiltration at concentrations of 50, 150, and 500 μM displayed increased auxinic response ranging from mild to more extreme epinasty relative to the concentration of 2,4-D applied. Both 2,4-D conjugates displayed similar response to 2,4-D at the respective concentration levels applied.

### Example 11

### Spot Infiltration Method

Examples 9 and 10 utilized a spot infiltration method for the delivery of an herbicide directly to leaves of a plant. The method generally allows for the directed delivery of a variety of agents to a localized area of a plant, e.g. localized region of leaves, for screening or assessment purposes, e.g. to quickly assess the response of a plant to the agent. As used in the foregoing examples, the spot infiltration method was used to introduce specific herbicides or specific conjugates of the herbicides to seedling plant leaves. Following introduction of the herbicide or the appropriate conjugate, plants were observed daily for nine days for characteristics of epinasty, trifoliate leaf curly, chlorosis, and new growth recovery.

Briefly, a solution of the desired agent is prepared of the desired agent, e.g. as described above in Example 10, solutions (5% EtOH) of 1, 2.5 and 5 mM IAA. The solution is then drawn into a 1 mL BD™ slip-tip syringe, bulk, non-sterile syringe (e.g. Becton, Dickinson and Company, Product No. 301025) with 100 μL markings. The desired volume is then applied to the leaf, e.g. as described in Examples 9 and 10, with the tip of the syring against the leaf and positive pressure applied to the syringe plunger. Sufficient pressure is applied in order to infuse the area beneath the syringe opening, but not so much pressure as to cause a visible-to-the-eye puncture or hole to form in the leaf. Some run-off of solution around the syringe opening is expected. In the foregoing examples, two spots were applied to each of two leaves per seedling for soybean plants. Typically, about four plants were thus treated for each concentration of agent tested. In soybean, both spots per leaf were at the distal portion of the leaf about 1-2 cm apart. When the method was applied to Arabidopsis, two developmentally intermediate aged leaves were chosen per plant, and one spot infiltration was carried out per leaf.

Generally, for Arabidopsis, the two leaves were chosen from opposite sides of rosette. In general, the spot infiltration method is carried out on younger plants in order to minimize experimental footprint and to maintain developmental uniformity. However, the method is not limited to use on young plants and can be easily applied to older plants as determined by the goals of a particular experiment.

As can be appreciated by one skilled in the art, the spot infiltration method as described herein can be utilized in the essential features for young and old plants, leaves of various sizes, and for a variety of agents of interest to screening on plants. The agents can include herbicides, insecticides, growth inducers, growth inhibitors, and the like. Volumes and the size of the spot infiltrated can be easily scaled to larger or smaller plants as the needs of the experiment require by utilizing larger or smaller commercially available syringes. In some circumstances, it may be useful to custom fabricate a syringe with a particular size opening or volume. A variety of vehicle solutions can be used for the agent of interest, so long as the vehicle is not directly deleterious to the outcome of the experiment. For example, aqueous solutions can be unmodified or comprise additional components such as short-chain alcohols (e.g. ethanol), ionic or nonic detergents, salts, or lipids. Such additional components can be added to enhance spot infiltration efficiency and/or to increase the solubility of the particular agent that is assessed.

The article “a” and “an” are used herein to refer to one or more than one (i.e., to at least one) of the grammatical object of the article. By way of example, “an element” means one or more element.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. A method to detoxify an auxin-analog herbicide comprising applying to a plant, a plant cell or a seed an auxin-analog herbicide, wherein said plant, plant cell or seed comprises a heterologous polynucleotide encoding a GH3 polypeptide having amino acid/auxin-analog conjugation activity, and wherein expression of the GH3 polypeptide produces an aspartate/auxin-analog conjugate or a glutamate/auxin-analog conjugate wherein said auxin-analog conjugate has reduced herbicidal activity,
and further wherein said GH3 polypeptide comprises
a) a polypeptide having an amino acid sequence having at least 85%, 90%, 95% or 100% sequence identity to any one of SEQ ID NO: 1, 2, 3, 4, 5, 6, 7, 8, 9, 10, 11, 12, 13, 14, 33, 34, 35, 36, 37, 38, 39, 40, 41, 42, 43, 44, 45, 46, 47, 48, 49, 50, 58, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 117, 118, 119, 120, 121, 124, 142, 144, or 145; or
b) a polypeptide having an amino acid sequence having at least 80%, 85%, 90%, 95% or 100% sequence identity to any one of SEQ ID NO: 15, 16, 17, 51, 52, 53, 54, 55, 56, 57, 59, 60, 61, 62, 63, 64, 65, 66, 67, 98, 99, 100, 101, 102, 103, 104, 105, 106, 107, 108, 109, 110, 111, 112,113, 114, 115, 116, 122, 134, 135, 136, 137, 138, 139, 140, or 141; or
c) a polypeptide having an amino acid sequence having at least 80%, 85%, 90%, 95% or 100% sequence identity to any one of SEQ ID NO: 18, 19, 20, 21, 22, 23, 24, 25, 26, 27, 28, 29, 30, 31, 32, 123, 125, 126, 127, 128, 129, 130, 131, 132, 133, or 143.

2. A method for controlling at least one weed in an area of cultivation comprising a crop or a seed of the crop, said method comprising applying to the area of cultivation and/or applying to the crop or a seed of the crop in the area of cultivation a sufficient amount of an auxin-analog herbicide to control weeds without significantly affecting the crop, wherein said crop or seed thereof in said area of cultivation comprises at least one heterologous polynucleotide encoding a GH3 polypeptide having amino acid/auxin-analog conjugation activity,
and further wherein said GH3 polypeptide comprises
a) a polypeptide having an amino acid sequence having at least 85%, 90%, 95% or 100% sequence identity to any one of SEQ ID NO: 1, 2, 3, 4, 5, 6, 7, 8, 9, 10, 11, 12, 13, 14, 33, 34, 35, 36, 37, 38, 39, 40, 41, 42, 43, 44, 45, 46, 47, 48, 49, 50, 58, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 117, 118, 119, 120, 121, 124, 142, 144, or 145; or
b) a polypeptide having an amino acid sequence having at least 80%, 85%, 90%, 95% or 100% sequence identity to any one of SEQ ID NO: 15, 16, 17, 51, 52, 53, 54, 55, 56, 57, 59, 60, 61, 62, 63, 64, 65, 66, 67, 98, 99, 100, 101, 102, 103, 104, 105, 106, 107, 108, 109, 110, 111, 112,113, 114, 115, 116, 122, 134, 135, 136, 137, 138, 139, 140, or 141; or
c) a polypeptide having an amino acid sequence having at least 80%, 85%, 90%, 95% or 100% sequence identity to any one of SEQ ID NO: 18, 19, 20, 21, 22, 23, 24, 25, 26, 27, 28, 29, 30, 31, 32, 123, 125, 126, 127, 128, 129, 130, 131, 132, 133, or 143.

3. A method for controlling at least one weed in an area of cultivation containing a crop comprising
(a) applying to the area of cultivation a sufficient amount of an auxin-analog herbicide to control weeds without significantly affecting the crop;
(b) planting the field with a crop or a seed thereof having a heterologous polynucleotide encoding a GH3 polypeptide having amino acid/auxin-analog conjugation activity,
and further wherein said GH3 polypeptide comprises
a) a polypeptide having an amino acid sequence having at least 85%, 90%, 95% or 100% sequence identity to any one of SEQ ID NO: 1, 2, 3, 4, 5, 6, 7, 8, 9, 10, 11, 12, 13, 14, 33, 34, 35, 36, 37, 38, 39, 40, 41, 42, 43, 44, 45, 46, 47, 48, 49, 50, 58, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 117, 118, 119, 120, 121, 124, 142, 144, or 145; or
b) a polypeptide having an amino acid sequence having at least 80%, 85%, 90%, 95% or 100% sequence identity to any one of SEQ ID NO: 15, 16, 17, 51, 52, 53, 54, 55, 56, 57, 59, 60, 61, 62, 63, 64, 65, 66, 67, 98, 99, 100, 101, 102, 103, 104, 105, 106, 107, 108, 109, 110, 111, 112,113, 114, 115, 116, 122, 134, 135, 136, 137, 138, 139, 140, or 141; or
c) a polypeptide having an amino acid sequence having at least 80%, 85%, 90%, 95% or 100% sequence identity to any one of SEQ ID NO: 18, 19, 20, 21, 22, 23, 24, 25, 26, 27, 28, 29, 30, 31, 32, 123, 125, 126, 127, 128, 129, 130, 131, 132, 133, or 143.

4. The method of any one of claim 2 or 3, wherein said crop exhibits an increased insensitivity to the auxin-analog herbicide as compared to an appropriate control plant.

5. (canceled)

6. (canceled)

7. (canceled)

8. (canceled)

9. The method of any one of claims 1-3, wherein
said auxin-analog herbicide comprises 2,4-D or dicamba.

10. The method of claim 2 or 3, wherein said crop further comprises at least one polypeptide imparting tolerance to an additional herbicide.

11. The method of claim 10, wherein said at least one polypeptide imparting tolerance to an additional herbicide comprises:
(a) a sulfonylurea-tolerant acetolactate synthase;
(b) an imidazolinone-tolerant acetolactate synthase;
(c) a glyphosate-tolerant 5-enolpyruvylshikimate-3-phosphate synthase;
(d) a glyphosate-tolerant glyphosate oxido-reductase;
(e) a glyphosate-N-acetyltransferase;
(f) a phosphinothricin acetyl transferase;
(g) a protoporphyrinogen oxidase;
(h) an HPPD enzyme;
(i) a P450 polypeptide; or,
(j) an acetyl coenzyme A carboxylase (ACCase).

12. The method of claim 11, wherein said at least one polypeptide imparting tolerance to an additional herbicide comprises a high resistance allele of acetolactate synthase (HRA) and/or a glyphosate-N-acetyltransferase polypeptide.

13. The method of claim 2 or 3, wherein the crop further comprises at least one additional polypeptide imparting tolerance to an auxin-analog herbicide, wherein said additional polypeptide is either the same or different than said GH3 polypeptide encoded by the heterologous polynucleotide.

14. The method of claim 1, wherein said plant, plant cell or seed is from a monocot.

15. The method of claim 1, wherein said plant, plant cell or seed is from a dicot.

16. The method of claim 2 or 3, wherein said crop is from a monocot.

17. The method of claim 2 or 3, wherein said crop is from a dicot.

18. (canceled)

19. (canceled)

20. (canceled)

21. (canceled)

22. (canceled)

23. (canceled)

24. (canceled)

25. (canceled)
